(19) United States
(12) Patent Application Publication
(10) Pub. No.: US 2024/0132624 A1
(43) Pub. Date: Apr. 25, 2024

(54) POLYPEPTIDES BINDING TO A SPECIFIC EPITOPE OF THE NEONATAL FC RECEPTOR

(71) Applicants: Ablynx N.V., Zwijnaarde (BE); Sanofi, Paris (FR)

(72) Inventors: Tom Van Bogaert, Gent (BE); Judith Verhelst, Gent (BE); Stephanie Staelens, Gent (BE); Carlo Boutton, Gent (BE); Wilbert De Witte, Gent (BE); Thomas Bertrand, Paris (FR)

(21) Appl. No.: 18/360,393

(22) Filed: Jul. 27, 2023

(30) Foreign Application Priority Data
Jul. 27, 2022 (EP) 22306120.1

(51) Int. Cl. C07K 16/42; A61K 47/68; C07K 16/18

(52) U.S. Cl. CPC C07K 16/42; A61K 47/6873; C07K 16/18; C07K 2317/31; C07K 2317/569

(57) ABSTRACT

The present invention relates to polypeptides that are capable of binding to a specific epitope on the neonatal Fc receptor (FcRn). In particular, the present invention relates to novel and improved polypeptides comprising immunoglobulin single variable domains (ISVDs), such as heavy-chain single variable domains, that are capable of binding to a specific epitope on FcRn. The invention further relates to constructs, compounds, molecules or chemical entities that comprise at least one of these ISVDs binding to a specific epitope on FcRn. The present invention further relates to methods for producing such polypeptides as well as to uses of such polypeptides for diverse applications, including but not limited to the extension of the half-life in vivo of therapeutic compounds and/or the prevention and/or treatment of a disease and/or disorder, such as but not limited to a proliferative disease, an inflammatory disease, an infectious disease or an autoimmune disease.

### 1. TECHNOLOGICAL FIELD

The present invention relates to polypeptides that are capable of binding to a specific epitope on the neonatal Fc receptor (FcRn).

In particular, the present invention relates to novel and improved polypeptides comprising immunoglobulin single variable domains (ISVDs), such as heavy-chain single variable domains, that are capable of binding to a specific epitope on FcRn. The invention further relates to constructs, compounds, molecules or chemical entities that comprise at least one of these ISVDs binding to a specific epitope on FcRn.

The present invention further relates to methods for producing such polypeptides as well as to uses of such polypeptides for diverse applications, including but not limited to the extension of the half-life in vivo of therapeutic compounds and/or the prevention and/or treatment of a disease and/or disorder, such as but not limited to a proliferative disease, an inflammatory disease, an infectious disease or an autoimmune disease.

### 2. TECHNOLOGICAL BACKGROUND

The neonatal Fc receptor (FcRn) is distinctively a beta (β)-2-microglobulin (β2m) associated protein that is structurally related to the major histocompatibility class I (MHC-1) family, yet it is unable to present antigenic peptides to T cells. FcRn is widely expressed with predominant expression in parenchymal, endothelial and hematopoietic cells. FcRn is predominantly localized intracellularly, although high surface levels have also been detected on immune cells such as monocytes or macrophages. FcRn has two known ligands, IgG and albumin, which together account for nearly 70% of total serum proteins.

While IgG subtypes are fundamental in immune responses, albumin functions as a carrier protein in addition to being an important regulator of oncotic blood pressure. Despite these differences, IgG and albumin have in common a long serum half-life owing to their interaction with FcRn, which rescues them from intracellular degradation through a cellular recycling mechanism. This recycling mechanism occurs in a strictly pH-dependent manner, with binding to FcRn taking place after uptake in the endosomal compartment at low pH (pH 5-6) and release at the cell surface at physiologic pH (which is typically a pH of about 7.4). Another of FcRn's functions is to transport IgG from mother to offspring, thereby providing to the naïve and immature immune system of the newborn the experience and protection developed in the adult progenitor. This process is developmentally regulated in that it occurs antenatally in rodents and humans through the inverted yolk sac or placenta, respectively, but uniquely continues at significant levels in the early post-natal life of rodents due to the high levels of FcRn expression in the intestinal epithelium. This functional expression of FcRn and its ability to transcytose IgG is not limited to the newborn but persists throughout life and permits the targeted delivery of IgG to sites where the presence of this type of antibody reinforces immunity, a process widely exploited by IgG-based therapeutics. Finally, the functions of FcRn are differentially determined by whether IgG is a single molecule, and thus monomeric, or present as an immune complex. In the latter case, FcRn has been shown to critically regulate the innate immune responses as well as processing and presentation of antigens contained within IgG immune complex.

Due to the many different important physiological roles of FcRn, there is a clear need for improved FcRn binding agents for therapeutic applications.

### 3. SUMMARY OF THE PRESENT TECHNOLOGY

The present inventors have identified improved FcRn binding polypeptides, which have several advantages over the FcRn binders as described in the prior art.

One of the advantageous characteristics of the FcRn binding polypeptides according to the present invention is that these bind to a unique epitope on FcRn, which epitope is different from the epitopes bound by the known natural FcRn ligands (i.e., serum albumin and IgG). Accordingly, the inventors have identified a novel epitope present on FcRn and have developed polypeptides capable of binding specifically to that epitope, without interfering with the natural physiological roles of FcRn. The polypeptides of the present invention can therefore be applied for several prophylactic, diagnostic and therapeutic applications in which biological pathways mediated by or involving FcRn play an important role, while avoiding the occurrence or at least limiting the severity of any potential side effects.

In a first aspect, the present invention thus provides polypeptides comprising at least one immunoglobulin single variable domain (ISVD) specifically binding to an epitope on FcRn, characterized in that the epitope comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

In certain particular embodiments, the polypeptides of the present invention are characterized in that the epitope comprises at least one of the following combinations of amino acid residues:
a) 4H and 5L, and/or
b) 98L, 99G, 100P, 101D and 102N, and/or
c) 167L, 171R, 174L, 175E and 177K, and/or
d) 255Q, 256H, 257A, 259L, 260A and 262P,
amino acid residues being numbered according to SEQ ID NO: 1.

In certain further particular embodiments, the polypeptides of the present invention are characterized in that the epitope at least comprises at least one of the following combinations of amino acid residues:
a) 2E, 3S, 4H and 5L, and/or
b) 97E, 98L, 99G, 100P, 101D and 102N, and/or
c) 98L, 99G, 100P, 101D, 102N and 103T, and/or
d) 167L, 168E, 171R, 174L, 175E and 177K, and/or
e) 205P, 206P and 207E, and/or
f) 255Q, 256H, 257A, 259L, 260A, 261Q, and 262P,
amino acid residues being numbered according to SEQ ID NO: 1.

In certain yet further particular embodiments, the polypeptides of the present invention are characterized in that the epitope comprises at least one of the following combinations of amino acid residues:
a) 1A, 2E, 3S, 4H and 5L, and/or
b) 164R, 167L, 168E, 171R, 174L, 175E and 177K, and/or
c) 204Y, 205P, 206P and 230E, and/or
d) 205P, 206P, 207E and 208L,
amino acid residues being numbered according to SEQ ID NO: 1.

According to certain embodiments, the polypeptides of the present invention are characterized in that said epitope at least comprises the following amino acid residues 4H, 5L, 98L, 99G, 100P, 101D, 102N, 167L, 171R, 174L, 175E, 177K, 207E, 255Q, 256H, 257A, 259L, 260A, and 262P, amino acid residues being numbered according to SEQ ID NO: 1.

According to further particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, amino acid residues being numbered according to SEQ ID NO: 1.

According to yet further particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P amino acid residues being numbered according to SEQ ID NO: 1.

In particular embodiments, the FcRn binding polypeptides of the present invention bind to the novel epitope on FcRn as disclosed herein in a pH-dependent manner, such that their binding affinity at an acidic pH, in particular at an acidic pH of between 5.0 and 6.8, is at least three times higher than the binding affinity at a neutral or physiological pH of about 7.4. In these particular embodiments, the polypeptides of the present invention show conditional specific binding to FcRn and as a result, by making use of the in vivo FcRn-mediated recycling mechanism, have a prolonged in vivo serum half-life. The polypeptides of the present invention can therefore be used to extend the in vivo half-life of therapeutic targets or therapeutic molecules of interest to which they are suitably linked, bound or fused.

In certain particular embodiments, the polypeptides of the present invention have a molecular weight of at least 30 kDa, and in particular have a molecular weight of between 30 kDa and 100 kDa. According to these particular embodiments, the compounds of the present invention can be most effectively and optimally applied for several applications, in particular for in vivo half-life extension of therapeutic molecules.

According to certain particular embodiments, the polypeptides of the present invention comprise at least one immunoglobulin single variable domain (ISVD) specifically binding to an epitope on FcRn characterized in that the epitope on FcRn bound by the polypeptides is different from the epitope on FcRn bound by serum albumin.

According to certain particular embodiments, the polypeptides of the present invention comprise at least one immunoglobulin single variable domain (ISVD) specifically binding to an epitope on FcRn characterized in that the epitope on FcRn bound by the polypeptides is different from the epitope on FcRn bound by immunoglobulin G (IgG).

In certain particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
a) CDR1 (according to Kabat) has the amino acid sequence of SEQ ID NO: 8 [=S Y A M Y] and/or has an amino acid sequence having 4, 3, 2 or only 1 “amino acid difference(s)” (as defined herein) with the sequence of SEQ ID NO: 8; and
b) CDR2 (according to Kabat) has the amino acid sequence of SEQ ID NO: 9 [=A I S S G G G S T D Y A D S V K G] and/or has an amino acid sequence having 4, 3, 2 or only 1 “amino acid difference(s)” (as defined herein) with the sequence of SEQ ID NO: 9; and
c) CDR3 (according to Kabat) has the amino acid sequence of SEQ ID NO: 10 [=D T L Y T S L T S Y S Y] and/or has an amino acid sequence having 4, 3, 2 or only 1 “amino acid difference(s)” (as defined herein) with the sequence of SEQ ID NO: 10.

In certain particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13.

In particular embodiments, the FcRn binding polypeptides of the present invention are characterized in that the at least one ISVD specifically binding to the epitope on FcRn as disclosed herein has the sequence of SEQ ID NO: 14 or SEQ ID NO: 15, or a sequence that has a sequence identity of 90%, such as 95% with the sequence of SEQ ID NO: 14 or SEQ ID NO: 15.

In particular embodiments, the polypeptides according to the invention specifically bind to FcRn, such as but not limited to human FcRn or cyno FcRn, with a dissociation constant (K_{D}) of between 103 nM and 10⁻² nM or less. Preferably, the K_{D} is determined by Kinexa, BLI or SPR, for instance as determined by SPR.

In particular embodiments, the polypeptides according to the invention specifically bind to FcRn with an affinity (K_{A}) of between 10⁻³ nM⁻¹ and 10² nM⁻¹.

In particular embodiments, the polypeptides according to the invention specifically bind to FcRn with an on-rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶ M⁻¹s⁻¹, at least about 10⁷ M⁻¹s⁻¹, and at least about 10⁸ M⁻¹s⁻¹, preferably as measured by surface plasmon resonance or BLI.

In particular embodiments, the polypeptides according to the invention specifically bind to FcRn with an off-rate constant (k_{off}) selected from the group consisting of at most about 10⁻¹s⁻¹, at most about 10⁻² s⁻¹, at most about 10⁻³s⁻¹, of at most about 10⁻⁴s⁻¹, at most about 10⁻⁵s⁻¹, and at most about 10⁻⁶s⁻¹, preferably as measured by surface plasmon resonance or BLI.

In certain particular embodiments, the present invention provides polypeptides characterized in that the at least one ISVD binding specifically to FcRn consists of 4 framework regions (FR1 to FR4 respectively) and 3 complementarity determining regions (CDR1 to CDR3 respectively), and is characterized in that:
a) CDR1 (according to Kabat) has the amino acid sequence of SEQ ID NO: 8 [=S Y A M Y] and/or has an amino acid sequence having 4, 3, 2 or only 1 “amino acid difference(s)” (as defined herein) with the sequence of SEQ ID NO: 8; and
b) CDR2 (according to Kabat) has the amino acid sequence of SEQ ID NO: 9 [=A I S S G G G S T D Y A D S V K G] and/or has an amino acid sequence having 4, 3, 2 or only 1 “amino acid difference(s)” (as defined herein) with the sequence of SEQ ID NO: 9; and
c) CDR3 (according to Kabat) has the amino acid sequence of SEQ ID NO: 10 [=D T L Y T S L T S Y S Y] and/or has an amino acid sequence having 4, 3, 2 or only 1 “amino acid difference(s)” (as defined herein) with the sequence of SEQ ID NO: 10.

In certain particular embodiments, the present invention provides polypeptides characterized in that the at least one ISVD binding specifically to FcRn consists of 4 framework regions (FR1 to FR4 respectively) and 3 complementarity determining regions (CDR1 to CDR3 respectively), and is characterized in that:
a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13.

In particular embodiments, the FcRn binding polypeptides of the present invention are characterized in that the at least one ISVD specifically binding to the epitope on FcRn as disclosed herein has the sequence of SEQ ID NO: 14 or SEQ ID NO: 15, or has a sequence identity of 90%, such as 95% with the sequence of SEQ ID NO: 14 or SEQ ID NO: 15.

In certain particular embodiments, the improved FcRn binding polypeptides of the present invention are such that they bind to FcRn in a pH-dependent manner.

In certain particular embodiments, the FcRn binding polypeptides of the present invention bind to a novel epitope on FcRn in a pH-dependent manner, such that the binding affinity at an acidic pH, in particular at an acidic pH of between 5.0 and 6.8, is at least three times higher than the binding affinity at a pH of about 7.4.

In particular embodiments, at an acidic pH of between 5.0 and 6.8, the polypeptides according to the invention specifically bind to FcRn with an affinity (K_{A}) of between 10³ nM⁻¹ and 10² nM⁻¹. The affinity (K_{A}) of these polypeptides for FcRn at acidic pH, preferably at a pH of between 5.0 and 6.8 is at least three times higher than the affinity (K_{A}) of the same polypeptides for FcRn at neutral or physiologic pH of about 7.4. In further particular embodiments, at an acidic pH of between 5.0 and 6.8, the polypeptides according to the invention bind to FcRn with an affinity that is at least ten times higher than the affinity (K_{A}) for FcRn of the same polypeptides at neutral or physiologic pH of about 7.4. In yet further particular embodiments, at an acidic pH of between 5.0 and 6.8, the polypeptides according to the invention bind to FcRn with an affinity that is at least fifty times higher, such as at least hundred times higher than the affinity (K_{A}) for FcRn of the same polypeptides at neutral or physiologic pH of about 7.4.

In certain particular embodiments, the present invention provides polypeptides as described herein characterized in that the at least one ISVD binds to FcRn at neutral or physiologic pH of about 7.4 with an affinity that is at least three times, such as at least ten times, such as at least fifty times, such as at least hundred times lower than the affinity with which the at least one ISVD binds to FcRn at acidic pH of between 5.0 and 6.8.

In a particular embodiment, at physiologic pH such as at a pH of about 7.4, the at least one ISVD binds to FcRn with a K_{A} value lower than 10⁴ liters/mol.

In certain particular embodiments, the present invention provides polypeptides as described herein characterized in that the at least one ISVD does not detectably, selectively or specifically bind to FcRn, or exhibits no or essentially no binding to FcRn at neutral or physiologic pH, such as at a pH of about 7.4.

In particular embodiments, at an acidic pH of between 5.0 and 6.8, the polypeptides according to the invention specifically bind to FcRn with a dissociation constant (K_{D}) of between 103 nM and 10⁻² nM or less. Preferably, the K_{D} is determined by Kinexa, BL or SPR, for instance as determined by SPR.

In particular embodiments, at an acidic pH of between 5.0 and 6.8, the polypeptides according to the invention specifically bind to FcRn with an on rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶ M⁻¹s⁻¹, at least about 10⁷ M⁻¹s⁻¹, and at least about 10⁸ M⁻¹s⁻¹, preferably as measured by surface plasmon resonance or BLI.

In particular embodiments, at an acidic pH of between 5.0 and 6.8, the polypeptides according to the invention specifically bind to FcRn with an off rate constant (k_{off}) selected from the group consisting of at most about 10⁻¹s⁻¹, at most about 10⁻²s⁻¹, at most about 10⁻³ s⁻¹, of at most about 10⁻⁴ s⁻¹, at most about 10⁻⁵ s⁻¹, and at most about 10⁻⁶ s⁻¹, preferably as measured by surface plasmon resonance or BLI.

The polypeptides of the present invention bind to a specific unique and novel epitope on FcRn, which is an epitope that is different from the epitopes bound by the natural ligands of FcRn, i.e., serum albumin and IgG.

In particular embodiments, the FcRn binding polypeptides according to the present invention are preferably such that when these are bound to or otherwise associated with an FcRn molecule, the binding of the FcRn molecule to serum albumin and/or IgG is not (significantly) affected, reduced or inhibited. In this particular embodiment, in a cross-blocking assay (as described herein), when the FcRn binding polypeptide is bound to or otherwise associated with an FcRn molecule, the displacement of serum albumin is less than 40%, such as less than 30%, less than 20%, less than 10% or essentially no displacement is detected (e.g., in ELISA or Alphascreen based competition assay). In this particular embodiment, in a cross-blocking assay (as described herein), when the FcRn binding polypeptide is bound to or otherwise associated with an FcRn molecule, the displacement of IgG is less than 40%, such as less than 30%, less than 20%, less than 10% or essentially no displacement is detected (e.g., in ELISA or Alphascreen based competition assay).

In further particular embodiments, the present invention provides polypeptides characterized in that they comprise at least one ISVD, which specifically binds to amino acid residues on FcRn that are not involved in binding of FcRn to serum albumin and/or to IgG. In this particular embodiment, the polypeptides comprise at least one ISVD which specifically binds to an epitope on FcRn that comprises amino acid residues that are not comprised in the epitope on FcRn bound by serum albumin and/or in the epitope on FcRn bound by IgG. In this particular embodiment, the polypeptides comprise at least one ISVD which specifically binds to amino acid residues on FcRn that are not bound by serum albumin and/or by IgG.

According to particular embodiments, the FcRn binding polypeptides of the invention are preferably also such that they compete with a polypeptide with the amino acid sequence of SEQ ID NO: 14 and/or SEQ ID NO: 15 for binding to FcRn and/or that they “cross-block” (as defined herein) the binding by the polypeptide with the amino acid sequence of SEQ ID NO: 14 and/or SEQ ID NO: 15 to FcRn.

In particular embodiments, the FcRn binding polypeptides of the invention are such that they bind to essentially the same amino acid residues and/or epitope on FcRn as bound by SEQ ID NO: 14 and/or SEQ ID NO: 15, and even more preferably such that they share essentially the same amino acid interactions as those of SEQ ID NO: 14 and/or SEQ ID NO: 15 with FcRn. For this purpose, according to a specific but non-limiting aspect, FcRn binding polypeptides according to the present invention preferably either have the same CDRs as the sequence of SEQ ID NO: 14 and/or SEQ ID NO: 15, or compared to the sequence of SEQ ID NO: 14 and/or SEQ ID NO: 15 preferably contain within their CDR's only such mutations (such as conservative amino acid substitutions) that still allow them to undergo the same or essentially the same amino acid interactions with FcRn as SEQ ID NO: 14 and/or SEQ ID NO: 15.

In certain particular embodiments, the present invention provides polypeptides as described herein characterized in that the ISVD is a (single) domain antibody, a Nanobody® V_{HH}, a V_{HH}, a humanized V_{HH}, or a camelized V_{H}.

In particular embodiments, the present invention provides polypeptides comprising: at least one ISVD specifically binding to FcRn in a pH-dependent manner, and at least one further moiety, characterized in that
a) the at least one ISVD specifically binds to FcRn in a pH-dependent manner, such that the binding affinity at a pH between 5.0 and 6.8 is at least three times higher than the binding affinity at a pH of about 7.4, and
b) the polypeptides have a molecular weight of at least 30 kDa.

In certain further particular embodiments, the polypeptides of the present invention have a molecular weight of between about 30 kDa and 200 kDa, such as between about 30 kDa and 100 kDa.

In certain further particular embodiments, the at least one further moiety is a protein moiety, such as a serum protein.

In certain further particular embodiments, the at least one further moiety is a protein binding moiety, such as a serum protein binding moiety.

In certain further particular embodiments, the at least one further moiety is a serum protein binding moiety, such as a serum albumin binding moiety.

In certain further particular embodiments, the at least one serum albumin binding moiety specifically binds to amino acid residues on serum albumin that are not involved in binding of serum albumin to FcRn.

In certain further particular embodiments, the present invention provides polypeptides as described herein, characterized in that the at least one serum albumin binding moiety is at least one ISVD specifically binding to serum albumin, such as a (single) domain antibody, a Nanobody® V_{HH}, a V_{H}H, a humanized V_{HH}, or a camelized V_{H}.

In certain further particular embodiments, the at least one further moiety is at least one ISVD binding to serum albumin.

In certain further particular embodiments, the at least one further moiety is at least one ISVD binding to serum albumin, which essentially consists of 4 framework regions (FR1 to FR4, respectively) and 3 complementarity determining regions (CDR1 to CDR3 respectively), and in which CDR1 is SFGMS (SEQ ID NO: 16), CDR2 is SISGSGSDTLYADSVKG (SEQ ID NO: 17) and CDR3 is GGSLSR (SEQ ID NO: 18), CDR determined according to Kabat definition; and/or in which CDR1 is GFTFRSFGMS (SEQ ID NO: 19), CDR2 is SISGSGSDTL (SEQ ID NO: 20) and CDR3 is GGSLSR (SEQ ID NO: 21), CDR determined according to AbM definition (Kontermann et al., 2010).

In particular embodiments, the at least one further moiety is an Fc region of an immunoglobulin (Ig). Thus, in certain particular embodiments, the invention provides at least one ISVD specifically binding to FcRn in a pH dependent manner, and at least one further moiety, characterized in that:
a) the at least one ISVD specifically binds to FcRn in a pH-dependent manner, such that the binding affinity at a pH between 5.0 and 6.8 is at least three times higher than the binding affinity at a pH of about 7.4,
b) the polypeptide has a molecular weight of at least 30 k Da, in particular between about 30 k Da and 100 kDa, and
c) the at least one further moiety is an Fc region or an Fc domain of an immunoglobulin (Ig).

In particular embodiments, the at least one further moiety is a non-protein moiety, such as but not limited to a poly-ethylene-glycol (PEG) moiety.

Thus, in certain particular embodiments, the invention provides at least one ISVD specifically binding to FcRn in a pH dependent manner, and at least one further moiety, characterized in that:
a) the at least one ISVD specifically binds to FcRn in a pH-dependent manner, such that the binding affinity at a pH between 5.0 and 6.5 is at least three times higher than the binding affinity at a pH of about 7.4,
b) the polypeptide has a molecular weight of at least 30 kDa, in particular between about 30 kDa and 100 kDa, and
c) the at least one further moiety is a non-protein moiety, such as but not limited to a poly-ethylene-glycol (PEG) moiety.

The polypeptide preferably further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.

In particular embodiments, the present invention provides polypeptides as described herein, characterized in that the polypeptides further comprise a therapeutic moiety.

In particular embodiments, the present invention provides polypeptides as described herein, characterized in that the polypeptides further comprise a therapeutic moiety, which comprises an ISVD such as a (single) domain antibody, a Nanobody® V_{HH}, a V_{HH}, a humanized V_{HH} or a camelized V_{H}.

In a further aspect, the present invention provides nucleic acids or nucleic acid sequences encoding polypeptides according to the present invention.

In another aspect, the present invention provides vectors comprising nucleic acids or nucleic acid sequences according to the present invention.

In yet another aspect, the present invention provides host cells transformed or transfected with the nucleic acids or nucleic acid sequences according to the present invention or with the vectors according to the present invention.

In a further aspect, the present invention provides a method or process for producing the polypeptides according to invention, said method at least comprising the steps of:
a. expressing, in a suitable (non-human) host cell or host organism or in another suitable expression system, a nucleic acid sequence; optionally followed by:
b. isolating and/or purifying the polypeptides according to the invention.

In yet a further aspect, the present invention provides pharmaceutical compositions comprising the polypeptides according to the present invention, or the polypeptides produced by the processes according to the present invention.

In a further aspect, the present invention provides polypeptides of the invention, or polypeptides produced according to the processes of the invention, for use in treating a subject in need thereof.

In a further aspect, the present invention provides methods for delivering a prophylactic or therapeutic polypeptide to a specific location, tissue or cell type in the body, the methods comprising the steps of administering to a subject, the polypeptides of the present invention, or produced by the processes according to the present invention.

In a further aspect, the present invention provides polypeptides of the present invention, or produced according to the processes of the present invention, for use in delivering a prophylactic or therapeutic polypeptide to a specific location, tissue or cell type in the body.

In yet a further aspect, the present invention provides polypeptides of the present invention, or produced according to the process of the present invention, for use in therapy.

In yet a further aspect, the present invention provides polypeptides of the present invention, or produced according to the process of the present invention, for use in the prevention, treatment or amelioration of a disease selected from the group consisting of a proliferative disease, an inflammatory disease, an infectious disease and an autoimmune disease.

In another aspect, the present invention provides methods for the prevention, treatment or amelioration of a disease selected from the group consisting of a proliferative disease, an inflammatory disease, an infectious disease and an autoimmune disease, comprising at least the step of administering to a subject in need thereof the polypeptide of the present invention, or produced by a method of the present invention.

In a further aspect, the present invention provides kits comprising polypeptides of the present invention, nucleic acids or nucleic acid sequences of the present invention, vectors of the present invention, or host cells of the present invention.

Unless indicated or defined otherwise, all terms used have their usual meaning in the art, which will be clear to the skilled person. Reference is for example made to the standard handbooks mentioned in paragraph a) on page 46 of WO 08/020079.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain, using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term.

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. However, for the following parameters, the specific margins shall apply: a temperature specified in ° C. with no decimal place shall have an error margin of ±1° C. (e.g., a temperature value of about 50° C. means 50° C.±1° C.); a time indicated in hours shall have an error margin of 0.1 hours irrespective of the decimal places (e.g., a time value of about 1.0 hours means 1.0 hours±0.1 hours; a time value of about 0.5 hours means 0.5 hours±0.1 hours).

In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

### 4. BRIEF DESCRIPTION OF THE DRAWINGS

FIG. 1 Sequence of human FcRn, wherein amino acid residues being identified as involved (either directly or indirectly) in binding to at least one of the polypeptides of the present invention, are in bold and underlined.

FIGS. 2A-2D show the three-dimensional protein structure of the alpha-chain of FcRn (aka Fc Fragment of IgG Receptor and Transporter (FcGRT)) wherein (FIG. 2A) the epitope for FcRn binding polypeptides according to particular embodiments of the invention is dark-shaded and, (FIG. 2B) the epitope for the Fc domain of IgG and (FIG. 2C) the epitope for serum albumin are light-shaded. In (FIG. 2D), both the epitope for FcRn binding polypeptides according to particular embodiments of the invention (dark-shaded in center) and the epitope for the Fc domain of IgG (light-shaded at right side) and the epitope for serum albumin (light-shaded at left side and upper center) are shown in combination.

FIGS. 3A-3D show the three-dimensional protein structure of hetero-dimeric FcRn (aka Fc Fragment of IgG Receptor and Transporter (FcGRT) and beta-2-microglobulin) wherein (FIG. 3A) the epitope for FcRn binding polypeptides according to particular embodiments of the invention is dark-shaded and, (FIG. 3B) the epitope for the Fc domain of IgG and (FIG. 3C) the epitope for serum albumin are light-shaded. In (FIG. 3D), both the epitope for FcRn binding polypeptides according to particular embodiments of the invention (dark-shaded in center) and the epitope for the Fc domain of IgG (light-shaded at right side) and the epitope for serum albumin (light-shaded at left side and upper center) are shown in combination.

FIG. 4 Mean (+/−SD, n=2) serum concentration-time profiles of different test bispecific ISVD constructs comprising an FcRn binding ISVD and an albumin binding ISVD (ALB23002) compared to control bispecific ISVD constructs comprising a control ISVD (IRR) and an albumin binding ISVD (ALB23002) following i.v. bolus administration at 1.9 mg/kg in female Tg32 mice.

FIG. 5 Mean (+/−SD, n=2) serum concentration-time profiles of test bispecific ISVD constructs, comprising the parental V_{HH} T0263018B11 or mutant variants thereof and an albumin binding ISVD (ALB23002) compared to control bispecific ISVD constructs comprising a control ISVD (IRR) and an albumin binding ISVD (ALB23002) following i.v. bolus administration at 1.9 mg/kg in female Tg32 mice.

FIG. 6 Mean (+/−SD, n=2) serum concentration-time profiles of multivalent ISVD test constructs, comprising an FcRn binding ISVD, an albumin-binding ISVD and additional ISVDs directed against potentially relevant therapeutic targets, following i.v. bolus administration in female Tg32 mice.

FIG. 7 Mean (+/−SD, n=2-3) serum concentration-time profiles of multivalent ISVD test constructs comprising one or more FcRn binding ISVDs and one or more control ISVDs, following i.v. bolus administration at 4.5 mg/kg in female Tg32 mice.

FIG. 8 Schematic drawing of the structural format of fusion polypeptide constructs as described in, e.g., Example 14. The Fc domains in the constructs were IgG4 FALA Fc backbone sequence variants with knob in hole mutations as described herein. One of these constructs (i.e., TP049) comprises additional amino acid differences or variations in the Fc backbone sequence (i.e., I253A, H310A, H435A), indicated as IgG Fc(IHH). The FcRn Nanobody® VHH used was in each case the T0263018B11 sequence as described herein (FcRn ISVD 1 in the figure). Nanobody® VHH not binding to FcRn or any other envisaged target is represented as “Control ISVD” in the figure. The Nanobody® VHH sequences in these fusion proteins were fused via a linker (as described in detail herein) to the N- and/or C-terminus of the Fc chain, i.e., via an IgG1 hinge (e.g., SEQ ID NO.: 126) and/or a GS linker (e.g., 35GS linker, SEQ ID NO.: 48), respectively, as described herein.

FIGS. 9A-9B Surface Plasmon Resonance (SPR) sensorgrams on the Biacore 8K+ instrument using different coating densities of FcRn (up to 5000 RU). The Nanobody® VHH-Fc proteins were characterized by affinity determination for human FcRn at pH 6.0 (FIG. 9A) and pH 7.4 (FIG. 9B).

FIG. 10 Mean (+/−SD, n=4-6) serum concentration-time profiles of ISVD-Fc fusion proteins, comprising an FcRn-binding ISVD and a Fc region of IgG4 FALA, following i.v. bolus administration in female Tg32 mice.

### 5. DETAILED DESCRIPTION

The present inventors have identified a unique epitope on FcRn, which is different to the epitope of the natural ligands of FcRn, such as serum albumin and IgG. In addition, and even more importantly, the present inventors have developed novel polypeptides capable of binding specifically to that epitope of FcRn, without interfering with the natural physiological roles of FcRn. The polypeptides as disclosed herein comprise at least one “immunoglobulin single variable domain” (ISVD) binding to human FcRn (SEQ ID NO: 1) or (polymorphic) variants or isoforms thereof. Isoforms are alternative protein sequences that can be generated from the same gene by a single biological event or by the combination of biological events such as alternative promoter usage, alternative splicing, alternative initiation and ribosomal frameshifting, all as known in the art.

Amino acid residues will be indicated interchangeably herein according to the standard three-letter or one-letter amino acid code, as mentioned in Table B-1 below.

TABLE B-1
Common amino acids

| 1-Letter Code | 3-Letter Code | Amino Acid Name |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides an amino acid sequence of a CDR wherein one (or more) of the amino acid residue(s) is (are) indicated with “X”, the description may further specify which amino acid residue(s) is (can be) present at that specific position of the CDR.

Amino acids are those L-amino acids commonly found in naturally occurring proteins and are listed in Table B-1. Those amino acid sequences containing D-amino acids are not intended to be embraced by this definition. Any amino acid sequence that contains post-translationally modified amino acids may be described as the amino acid sequence that is initially translated using the symbols shown in the Table B-1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition. The terms “protein”, “peptide”, “protein/peptide”, and “polypeptide” are used interchangeably throughout the disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide from a protein; that polypeptides may be made by chemical synthesis or recombinant methods; and that proteins are generally made in vitro or in vivo by recombinant methods as known in the art.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first-mentioned nucleotide sequence or amino acid sequence, respectively, but more usually this generally means that the first-mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first-mentioned sequence has actually been generated or obtained (which may for example be by any suitable method described herein). By means of a non-limiting example, when an ISVD is said to comprise a CDR sequence, this may mean that said CDR sequence has been incorporated into the ISVD, but more usually this generally means that the ISVD contains within its sequence a stretch of amino acid residues with the same amino acid sequence as said CDR sequence, irrespective of how said ISVD has been generated or obtained. It should also be noted that when the latter amino acid sequence has a specific biological or structural function, it preferably has essentially the same, a similar or an equivalent biological or structural function in the first-mentioned amino acid sequence (in other words, the first-mentioned amino acid sequence is preferably such that the latter sequence is capable of performing essentially the same, a similar or an equivalent biological or structural function). For example, when an ISVD is said to comprise a CDR sequence or framework sequence, respectively, the CDR sequence and framework are preferably capable, in said ISVD, of functioning as a CDR sequence or framework sequence, respectively. Also, when a nucleotide sequence is said to comprise another nucleotide sequence, the first-mentioned nucleotide sequence is preferably such that, when it is expressed into an expression product (e.g., a polypeptide), the amino acid sequence encoded by the latter nucleotide sequence forms part of said expression product (in other words, that the latter nucleotide sequence is in the same reading frame as the first-mentioned, larger nucleotide sequence).

The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.

In the context of the present technology, “binding to” a certain target molecule has the usual meaning in the art as understood in the context of antibodies and their respective antigens.

The epitope to which the polypeptides of the present invention are specifically directed is an epitope on FcRn, which comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to the order or numbering of the amino acid residues in the amino acid sequence of SEQ ID NO: 1.

The term “antigenic determinant” refers to the epitope on the antigen recognized by the antigen binding molecule (such as an ISVD or a polypeptide comprising the ISVD) and more in particular by the antigen binding site of said molecule. The terms “antigenic determinant” and “epitope’ may also be used interchangeably herein. The antigen binding molecule (such as an antibody, an ISVD, a polypeptide of the invention, or generally an antigen-binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.

In particular embodiments, the epitope as disclosed herein is a linear epitope, comprising a stretch of continuous amino acid residues of the FcRn primary sequence.

In particular embodiments, the epitope as disclosed herein is a conformational epitope, comprising at least two discontinuous amino acid residues and/or stretches of amino acid residues of the FcRn primary sequence that are close to each other in the FcRn tertiary structure and that form a specific binding surface with a three-dimensional structure for the polypeptides of the present invention.

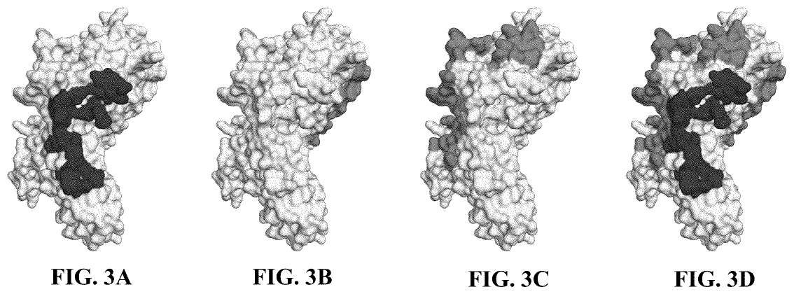

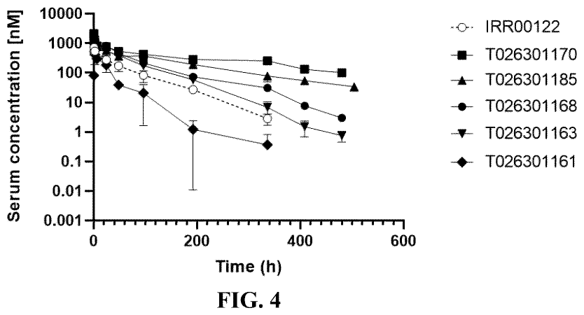

According to certain particular embodiments, the epitope on FcRn disclosed herein against which the polypeptides according to the present invention are directed, is characterized in that it is different from the epitope on FcRn against which serum albumin is directed (see FIGS. 2A-2D and 3A-3D).

According to certain particular embodiments, the epitope on FcRn disclosed herein against which the polypeptides according to the present invention are directed, is characterized in that it is different from the epitope on FcRn against which immunoglobulin G (IgG) is directed (see FIGS. 2A-2D and 3A-3D).

According to certain embodiments, the polypeptides of the present invention are characterized in that the epitope on FcRn to which they bind comprises at least one of the following combinations of amino acid residues:
a) 4H and 5L, and/or
b) 98L, 99G, 100P, 101D and 102N, and/or
c) 167L, 171R, 174L, 175E and 177K, and/or
d) 255Q, 256H, 257A, 259L, 260A and 262P,
amino acid residues being numbered according to the order or numbering of the amino acid residues in the amino acid sequence of SEQ ID NO: 1.

According to certain further embodiments, the polypeptides of the present invention are characterized in that the epitope to which they bind comprises at least one of the following combinations of amino acid residues:
a) 2E, 3S, 4H and 5L, and/or
b) 97E, 98L, 99G, 100P, 101D and 102N, and/or
c) 98L, 99G, 100P, 101D, 102N and 103T, and/or
d) 167L, 168E, 171R, 174L, 175E and 177K, and/or
e) 205P, 206P and 207E, and/or
f) 255Q, 256H, 257A, 259L, 260A, 261Q, and 262P,
amino acid residues being numbered according to the order or numbering of the amino acid residues in the amino acid sequence of SEQ ID NO: 1.

According to certain further embodiments, the polypeptides of the present invention are characterized in that the epitope to which they bind comprises at least one of the following combinations of amino acid residues:
a) 1A, 2E, 3S, 4H and 5L, and/or
b) 164R, 167L, 168E, 171R, 174L, 175E and 177K, and/or
c) 204Y, 205P, 206P and 230E, and/or
d) 205P, 206P, 207E and 208L,
amino acid residues being numbered according to the order or numbering of the amino acid residues in the amino acid sequence of SEQ ID NO: 1.

According to yet further embodiments, the polypeptides of the present invention are characterized in that the epitope to which they bind at least comprises the following amino acid residues 4H, 5L, 98L, 99G, 100P, 101D, 102N, 167L, 171R, 174L, 175E, 177K, 207E, 255Q, 256H, 257A, 259L, 260A, and 262P, amino acid residues being numbered according to the order or numbering of the amino acid residues in the amino acid sequence of SEQ ID NO: 1.

According to further particular embodiments, the polypeptides of the present invention are characterized in that the epitope to which they bind at least comprises the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, amino acid residues being numbered according to the order or numbering of the amino acid residues in the amino acid sequence of SEQ ID NO: 1.

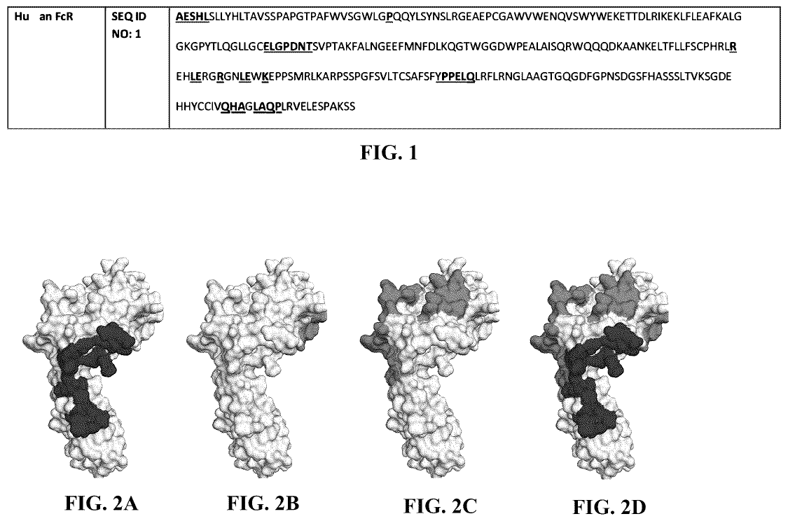

According to yet further particular embodiments, the polypeptides of the present invention are characterized in that the epitope to which they bind at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P amino acid residues being numbered according to the order or numbering of the amino acid residues in the amino acid sequence of SEQ ID NO: 1 (see FIGS. 1, 2A-2D, and 3A-3D).

According to other particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L and/or 260A, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and/or 259L, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H and/or 257A, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q and/or 255Q, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L and/or 209Q, amino acid residues being numbered according to SEQ ID NO: 1.

According to other particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L and/or 260A, amino acid residues being numbered according to SEQ ID NO: 1.

According to other particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and/or 259L, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and/or 259L, amino acid residues being numbered according to SEQ ID NO: 1.

According to other particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and/or 259L, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and/or 259L, amino acid residues being numbered according to SEQ ID NO:1.

According to other particular embodiments, the polypeptides of the present invention are characterized in that the epitope at least comprises the following amino acid residues 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H and/or 257A, amino acid residues being numbered according to SEQ ID NO:1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H and/or 257A, amino acid residues being numbered according to SEQ ID NO: 1.

According to other particular embodiments, the polypeptides of the present invention are characterized in that the epitope at least comprises the following amino acid residues 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H and/or 257A, amino acid residues being numbered according to SEQ ID NO: 1.

According to other particular embodiments, the polypeptides of the present invention are characterized in that the epitope at least comprises the following amino acid residues 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q and/or 256H, amino acid residues being numbered according to SEQ ID NO: 1.

According to other particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q and/or 256H, amino acid residues being numbered according to SEQ ID NO: 1.

According to other particular embodiments, the polypeptides of the present invention are characterized in that the epitope at least comprises the following amino acid residues 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to other particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q and/or 255Q, amino acid residues being numbered according to SEQ ID NO: 1.

According to other particular embodiments, the polypeptides of the present invention are characterized in that the epitope at least comprises the following amino acid residues: 4H, 5L, 98L, 99G, 100P, 101D, 102N, 167L, 171R, 174L, 175E, 177K, 255Q, 256H, 257A, 259L, 260A and 262P, amino acid residues being numbered according to SEQ ID NO: 1.

According to other particular embodiments, the polypeptides of the present invention are characterized in that the epitope at least comprises the following amino acid residues: 4H and 5L.

According to other particular embodiments, the polypeptides of the present invention are characterized in that the epitope at least comprises the following amino acid residues: 98L, 99G, 100P, 101D and 102N.

According to other particular embodiments, the polypeptides of the present invention are characterized in that the epitope at least comprises the following amino acid residues: 167L, 171R, 174L, 175E and 177K.

According to other particular embodiments, the polypeptides of the present invention are characterized in that the epitope at least comprises the following amino acid residues: 255Q, 256H, 257A, 259L, 260A and 262P.

According to other particular embodiments, the polypeptides of the present invention are characterized in that the epitope at least comprises the following amino acid residues: 4H, 5L, 167L, 171R, 174L, 175E and 177K.

According to other particular embodiments, the polypeptides of the present invention are characterized in that the epitope at least comprises the following amino acid residues: 4H, 5L, 98L, 99G, 100P, 101D and 102N.

According to other particular embodiments, the polypeptides of the present invention are characterized in that the epitope at least comprises the following amino acid residues: 4H, 5L, 255Q, 256H, 257A, 259L, 260A and 262P.

According to other particular embodiments, the polypeptides of the present invention are characterized in that the epitope at least comprises the following amino acid residues: 4H, 5L, 167L, 171R, 174L, 175E, 177K, 255Q, 256H, 257A, 259L, 260A and 262P.

According to other particular embodiments, the polypeptides of the present invention are characterized in that the epitope at least comprises the following amino acid residues: 167L, 171R, 174L, 175E, 177K, 255Q, 256H, 257A, 259L, 260A and 262P, According to other particular embodiments, the polypeptides of the present invention are characterized in that the epitope at least comprises the following amino acid residues: 98L, 99G, 100P, 101D, 102N, 167L, 171R, 174L, 175E, 177K, 255Q, 256H, 257A, 259L, 260A and 262P, amino acid residues being numbered according to SEQ ID NO: 1.

According to other particular embodiments, the polypeptides of the present invention are characterized in that the epitope at least comprises the following amino acid residues: 98L, 99G, 100P, 101D, 102N, 167L, 171R, 174L, 175E and 177K, amino acid residues being numbered according to SEQ ID NO: 1.

According to other particular embodiments, the polypeptides of the present invention are characterized in that the epitope at least comprises the following amino acid residues: 4H, 5L, 98L, 99G, 100P, 101D, 102N, 167L, 171R, 174L, 175E and 177K, amino acid residues being numbered according to SEQ ID NO: 1.

According to other particular embodiments, the polypeptides of the present invention are characterized in that the epitope at least comprises the following amino acid residues: 2E, 3S, 4H, 5L, 97E, 98L, 99G, 100P, 101D, 102N, 98L, 99G, 100P, 101D, 102N, 103T, 167L, 168E, 171R, 174L, 175E 177K, 205P, 206P, 207E, 255Q, 256H, 257A, 259L, 260A, 261Q, and 262P, amino acid residues being numbered according to SEQ ID NO: 1.

According to other particular embodiments, the polypeptides of the present invention are characterized in that the epitope at least comprises the following amino acid residues: 2E, 3S, 4H and 5L.

According to other particular embodiments, the polypeptides of the present invention are characterized in that the epitope at least comprises the following amino acid residues: 97E, 98L, 99G, 100P, 101D and 102N.

According to other particular embodiments, the polypeptides of the present invention are characterized in that the epitope at least comprises the following amino acid residues: 98L, 99G, 100P, 101D, 102N and 103T.

According to other particular embodiments, the polypeptides of the present invention are characterized in that the epitope at least comprises the following amino acid residues: 167L, 168E, 171R, 174L, 175E and 177K.

According to other particular embodiments, the polypeptides of the present invention are characterized in that the epitope at least comprises the following amino acid residues: 205P, 206P and 207E.

According to other particular embodiments, the polypeptides of the present invention are characterized in that the epitope at least comprises the following amino acid residues: 255Q, 256H, 257A, 259L, 260A, 261Q, and 262P.

According to other particular embodiments, the polypeptides of the present invention are characterized in that the epitope at least comprises the following amino acid residues: 2E, 3S, 4H, 5L 97E, 98L, 99G, 100P, 101D and 102N.

According to other particular embodiments, the polypeptides of the present invention are characterized in that the epitope at least comprises the following amino acid residues: 2E, 3S, 4H, 5L 167L, 168E, 171R, 174L, 175E and 177K.

According to other particular embodiments, the polypeptides of the present invention are characterized in that the epitope at least comprises the following amino acid residues: 2E, 3S, 4H, 5L, 205P, 206P and 207E.

According to other particular embodiments, the polypeptides of the present invention are characterized in that the epitope at least comprises the following amino acid residues: 2E, 3S, 4H, 5L 98L, 99G, 100P, 101D, 102N and 103T.

According to other particular embodiments, the polypeptides of the present invention are characterized in that the epitope at least comprises the following amino acid residues: 205P, 206P, 207E, 255Q, 256H, 257A, 259L, 260A, 261Q, and 262P.

According to other particular embodiments, the polypeptides of the present invention are characterized in that the epitope at least comprises the following amino acid residues: 98L, 99G, 100P, 101D, 102N, 103T, 167L, 168E, 171R, 174L, 175E, 177K, 205P, 206P and 207E.

According to other particular embodiments, the polypeptides of the present invention are characterized in that the epitope at least comprises the following amino acid residues: 97E, 98L, 99G, 100P, 101D, 102N, 167L, 168E, 171R, 174L, 175E, 177K, 255Q, 256H, 257A, 259L, 260A, 261Q, and 262P.

According to other particular embodiments, the polypeptides of the present invention are characterized in that the epitope at least comprises the following amino acid residues: 2E, 3S, 4H, 5L, 205P, 206P, 207E, 255Q, 256H, 257A, 259L, 260A, 261Q, and 262P.

According to other particular embodiments, the polypeptides of the present invention are characterized in that the epitope at least comprises the following amino acid residues: 2E, 3S, 4H, 5L, 97E, 98L, 99G, 100P, 101D, 102N, 255Q, 256H, 257A, 259L, 260A, 261Q, and 262P.

According to other particular embodiments, the polypeptides of the present invention are characterized in that the epitope at least comprises the following amino acid residues: 2E, 3S, 4H, 5L, 255Q, 256H, 257A, 259L, 260A, 261Q, and 262P.

According to other particular embodiments, the polypeptides of the present invention are characterized in that the epitope at least comprises the following amino acid residues: 2E, 3S, 4H, 5L, 97E, 98L, 99G, 100P, 101D, 102N, 167L, 168E, 171R, 174L, 175E, 177K, 255Q, 256H, 257A, 259L, 260A, 261Q, and 262P.

According to other particular embodiments, the polypeptides of the present invention are characterized in that the epitope at least comprises the following amino acid residues: 167L, 168E, 171R, 174L, 175E, 177K, 205P, 206P, 207E, 255Q, 256H, 257A, 259L, 260A, 261Q, and 262P.

According to other particular embodiments, the polypeptides of the present invention are characterized in that the epitope at least comprises the following amino acid residues: 97E, 98L, 99G, 100P, 101D, 102N, 255Q, 256H, 2579, 259L, 2609, 261Q, and 262P.

According to other particular embodiments, the polypeptides of the present invention are characterized in that the epitope at least comprises the following amino acid residues: 97E, 98L, 99G, 100P, 101D, 102N, 98L, 99G, 100P, 101D, 102N and 103T.

According to other particular embodiments, the polypeptides of the present invention are characterized in that the epitope at least comprises the following amino acid residues: 97E, 98L, 99G, 100P, 101D, 102N, 205P, 206P and 207E.

According to other particular embodiments, the polypeptides of the present invention are characterized in that the epitope at least comprises the following amino acid residues: 97E, 98L, 99G, 100P, 101D, 102N, 167L, 168E, 171R, 174L, 175E and 177K.

According to other particular embodiments, the polypeptides of the present invention are characterized in that the epitope at least comprises the following amino acid residues: 167L, 168E, 171R, 174L, 175E, 177K, 205P, 206P and 207E.

According to other particular embodiments, the polypeptides of the present invention are characterized in that the epitope at least comprises the following amino acid residues: 167L, 168E, 171R, 174L, 175E, 177K, 98L, 99G, 100P, 101D, 102N and 103T.

According to other particular embodiments, the polypeptides of the present invention are characterized in that the epitope at least comprises the following amino acid residues: 167L, 168E, 171R, 174L, 175E, 177K, 255Q, 256H, 257A, 259L, 260A, 261Q, and 262P.

According to other particular embodiments, the polypeptides of the present invention are characterized in that the epitope at least comprises the following amino acid residues: 205P, 206P, 207E, 255Q, 256H, 257A, 259L, 260A, 261Q, and 262P.

According to other particular embodiments, the polypeptides of the present invention are characterized in that the epitope at least comprises the following amino acid residues: 97E, 98L, 99G, 100P, 101D, 102N, 98L, 99G, 100P, 101D, 102N, 103T, 167L, 168E, 171R, 174L, 175E 177K, 205P, 206P, 207E, 255Q, 256H, 257A, 259L, 260A, 261Q, and 262P, amino acid residues being numbered according to SEQ ID NO: 1.

According to other particular embodiments, the polypeptides of the present invention are characterized in that the epitope at least comprises the following amino acid residues: 2E, 3S, 4H, 5L, 97E, 98L, 99G, 100P, 101D, 102N, 98L, 99G, 100P, 101D, 102N, 103T, 167L, 168E, 171R, 174L, 175E 177K, 205P, 206P and 207E, amino acid residues being numbered according to SEQ ID NO: 1.

According to other particular embodiments, the polypeptides of the present invention are characterized in that the epitope at least comprises the following amino acid residues: 97E, 98L, 99G, 100P, 101D, 102N, 98L, 99G, 100P, 101D, 102N, 103T, 167L, 168E, 171R, 174L, 175E 177K, 205P, 206P and 207E, amino acid residues being numbered according to SEQ ID NO: 1.

According to other particular embodiments, the polypeptides of the present invention are characterized in that the epitope at least comprises the following amino acid residues: 1A, 2E, 3S, 4H, 5L, 164R, 167L, 168E, 171R, 174L, 175E, 177K, 204Y, 205P, 206P, 230E, 205P, 206P, 207E, 208L, amino acid residues being numbered according to SEQ ID NO: 1.

According to other particular embodiments, the polypeptides of the present invention are characterized in that the epitope at least comprises the following amino acid residues: 1A, 2E, 3S, 4H and 5L.

According to other particular embodiments, the polypeptides of the present invention are characterized in that the epitope at least comprises the following amino acid residues: 164R, 167L, 168E, 171R, 174L, 175E and 177K.

According to other particular embodiments, the polypeptides of the present invention are characterized in that the epitope at least comprises the following amino acid residues: 204Y, 205P, 206P and 230E.

According to other particular embodiments, the polypeptides of the present invention are characterized in that the epitope at least comprises the following amino acid residues: 205P, 206P, 207E and 208L.

According to other particular embodiments, the polypeptides of the present invention are characterized in that the epitope at least comprises the following amino acid residues: 1A, 2E, 3S, 4H 5L, 164R, 167L, 168E, 171R, 174L, 175E and 177K.

According to other particular embodiments, the polypeptides of the present invention are characterized in that the epitope at least comprises the following amino acid residues: 1A, 2E, 3S, 4H 5L, 204Y, 205P, 206P and 230E.

According to other particular embodiments, the polypeptides of the present invention are characterized in that the epitope at least comprises the following amino acid residues: 1A, 2E, 3S, 4H 5L, 205P, 206P, 207E and 208L.

According to other particular embodiments, the polypeptides of the present invention are characterized in that the epitope at least comprises the following amino acid residues: 204Y, 205P, 206P, 230E, 205P, 206P, 207E and 208L.

According to other particular embodiments, the polypeptides of the present invention are characterized in that the epitope at least comprises the following amino acid residues: 1A, 2E, 3S, 4H, 5L 204Y, 205P, 206P, 230E, 205P, 206P, 207E and 208L.

According to other particular embodiments, the polypeptides of the present invention are characterized in that the epitope at least comprises the following amino acid residues: 164R, 167L, 168E, 171R, 174L, 175E, 177K, 204Y, 205P, 206P, 230E, 205P, 206P, 207E, 208L, amino acid residues being numbered according to SEQ ID NO: 1.

According to other particular embodiments, the polypeptides of the present invention are characterized in that the epitope at least comprises the following amino acid residues: 1A, 2E, 3S, 4H, 5L, 164R, 167L, 168E, 171R, 174L, 175E, 177K, 204Y, 205P, 206P and 230E, amino acid residues being numbered according to SEQ ID NO: 1.

According to other particular embodiments, the polypeptides of the present invention are characterized in that the epitope at least comprises the following amino acid residues: 1A, 2E, 3S, 4H, 5L, 164R, 167L, 168E, 171R, 174L, 175E, 177K, 204Y, 205P, 206P and 230E, amino acid residues being numbered according to SEQ ID NO: 1.

According to certain embodiments, the polypeptides of the present invention are characterized in that said epitope at least comprises the following amino acid residues 5L, 98L, 99G, 100P, 101D, 102N, 167L, 171R, 174L, 175E, 177K, 207E, 255Q, 256H, 257A, 259L, 260A, and 262P, amino acid residues being numbered according to SEQ ID NO: 1.

According to certain embodiments, the polypeptides of the present invention are characterized in that said epitope at least comprises the following amino acid residues 4H, 5L, 98L, 99G, 100P, 101D, 102N, 167L, 171R, 174L, 175E, 177K, 207E, 255Q, 256H, 257A, 259L and 260A, amino acid residues being numbered according to SEQ ID NO: 1.

According to certain embodiments, the polypeptides of the present invention are characterized in that said epitope at least comprises the following amino acid residues 98L, 99G, 100P, 101D, 102N, 167L, 171R, 174L, 175E, 177K, 207E, 255Q, 256H, 257A, 259L, 260A, and 262P, amino acid residues being numbered according to SEQ ID NO: 1.

According to certain embodiments, the polypeptides of the present invention are characterized in that said epitope at least comprises the following amino acid residues 4H, 5L, 98L, 99G, 100P, 101D, 102N, 167L, 171R, 174L, 175E, 177K, 207E, 255Q, 256H, 257A and 259L, amino acid residues being numbered according to SEQ ID NO: 1.

According to certain embodiments, the polypeptides of the present invention are characterized in that said epitope at least comprises the following amino acid residues 98L, 99G, 100P, 101D, 102N, 167L, 171R, 174L, 175E, 177K, 207E, 255Q, 256H, 257A and 259L, amino acid residues being numbered according to SEQ ID NO: 1.

According to further particular embodiments, the polypeptides of the present invention are characterized in that the epitope at least comprises the following amino acid residues 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, amino acid residues being numbered according to SEQ ID NO: 1.

According to further particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A and 261Q, amino acid residues being numbered according to SEQ ID NO: 1.

According to yet further particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P amino acid residues being numbered according to SEQ ID NO: 1.

According to yet further particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and 261Q, amino acid residues being numbered according to SEQ ID NO: 1.

According to yet further particular embodiments, the polypeptides of the present invention are characterized in that the epitope at least comprises the following amino acid residues 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and 261Q, amino acid residues being numbered according to SEQ ID NO: 1.

According to yet further particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P amino acid residues being numbered according to SEQ ID NO: 1.

According to yet further particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L and 260Aamino acid residues being numbered according to SEQ ID NO: 1.

According to yet further particular embodiments, the polypeptides of the present invention are characterized in that the epitope at least comprises the following amino acid residues 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L and 260A, amino acid residues being numbered according to SEQ ID NO: 1.

According to yet further particular embodiments, the polypeptides of the present invention are characterized in that the epitope at least comprises the following amino acid residues 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and 262P amino acid residues being numbered according to SEQ ID NO: 1.

According to yet further particular embodiments, the polypeptides of the present invention are characterized in that the epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A and 259L amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, and 260A, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, and/or 262P, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A and/or 261Q, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H and/or 257A, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q and/or 256H, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q and/or 255Q, amino acid residues being numbered according to SEQ ID NO: 1.

According to yet further particular embodiments, the polypeptides of the present invention are characterized in that the epitope at least comprises the following amino acid residues: 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L and/or 209Q, amino acid residues being numbered according to SEQ ID NO: 1.

The polypeptides according to the invention specifically bind to the FcRn epitope as disclosed herein through at least one immunoglobulin single variable domain (ISVD) that interacts with that epitope on FcRn.

### 5.1 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® molecule (as defined herein, and including but not limited to a V_{HH}); other single variable domains, or any suitable fragment of any one thereof.

In particular, the immunoglobulin single variable domain may be a Nanobody® immunoglobulin single variable domain (such as a V_{HH}, including a humanized V_{HH} or camelized V_{H}) or a suitable fragment thereof. [Note: Nanobody® is a registered trademark of Ablynx N.V.]“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al., Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}s, reference is made to the review article by Muyldermans (Reviews in Molecular Biotechnology 74: 277-302, 2001).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries, e.g., by phage display.

The generation of immunoglobulin sequences, such as Nanobody® V_{HH}s, has been described extensively in various publications, among which WO 94/04678, Hamers-Casterman et al. 1993 and Muyldermans et al., 2001 (Reviews in Molecular Biotechnology 74: 277-302, 2001) can be exemplified. In these methods, camelids are immunized with the target antigen in order to induce an immune response against said target antigen. The repertoire of V_{HH}s obtained from said immunization is further screened for V_{HH}s that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The technology also includes fully human, humanized or chimeric sequences. For example, the invention comprises camelid immunoglobulin sequences and humanized camelid immunoglobulin sequences, or camelized domain antibodies, e.g., camelized dAb as described by Ward et al. (see for example WO 94/04678 and Riechmann, Febs Lett., 339:285-290, 1994 and Prot. Eng., 9:531-537, 1996). Moreover, the invention also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g., toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized V_{HH}s can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e., by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g., WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

As further described in paragraph q) on pages 58 and 59 of WO 08/020079 (incorporated herein by reference), the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIGS. 2A-2D of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dubel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the invention may contain one or more of hallmark residues (as defined herein), such that the ISVD sequence is a Nanobody® molecule, such as a V_{HH}, including a humanized V_{HH} or camelized V_{H}. Some preferred, but non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the invention is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences.

In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known perse.

As described above, an ISVD may be a Nanobody® V_{HH} or a suitable fragment thereof. For a general description of ISVDs, reference is made to the further description below, as well as to the prior art cited herein. In this respect, it should however be noted that this description and the prior art mainly described ISVDs of the so-called “V_{H}3 class” (i.e., ISVDs with a high degree of sequence homology to human germline sequences of the V_{H}3 class such as DP-47, DP-51 or DP-29). It should however be noted that the invention in its broadest sense can generally use any type of ISVD, and for example also uses the ISVDs belonging to the so-called “V_{H}4 class” (i.e., ISVDs with a high degree of sequence homology to human germline sequences of the V_{H}4 class such as DP-78), as for example described in WO 2007/118670.

Generally, ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein).

Thus, generally, a ISVD can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
   one or more of the amino acid residues at positions 11, 37, 44, 45, 47, 83, 84, 103, 104 and 108 according to the Kabat numbering are chosen from the Hallmark residues mentioned in Table A-0 below.

TABLE A-0
Hallmark Residues in Nanobody ® ISVDs

| Position | Human V_{H}3 | Hallmark Residues |
|---|---|---|
| 11 | L, V; | L, S, V, M, W, F, T, Q, E, A, R, |
| | predominantly L | G, K, Y, N, P, I; preferably L |
| 37 | V, I, F; | F⁽¹⁾, Y, V, L, A, H, S, I, W, C, |
| | usually V | N, G, D, T, P, preferably F⁽¹⁾ or Y |
| 44⁽⁸⁾ | G | E⁽³⁾, Q⁽³⁾, G⁽²⁾, D, A, K, R, L, |
| | | P, S, V, H, T, N, W, M, I; |
| | | preferably G⁽²⁾, E⁽³⁾ or Q⁽³⁾; |
| | | most preferably G⁽²⁾ or Q⁽³⁾. |
| 45⁽⁸⁾ | L | L⁽²⁾, R⁽³⁾, P, H, F, G, Q, S, E, |
| | | T, Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, |
| | | M, R, Y, E, P, T, C, H, K, Q, N, D; |
| | | preferably W⁽²⁾, L⁽¹⁾ or F⁽¹⁾ |
| 83 | R or K; | R, K⁽⁵⁾, T, E⁽⁵⁾, Q, N, S, I, V, G, |
| | usually R | M, L, A, D, Y, H; preferably K or R; |
| | | most preferably K |
| 84 | A, T, D; | P⁽⁵⁾, S, H, L, A, V, I, T, F, D, R, |
| | predominantly A | Y, N, Q, G, E; preferably P |
| 103 | W | W⁽⁴⁾, R⁽⁶⁾, G, S, K, A, M, Y, L, F, |
| | | T, N, V, Q, P⁽⁶⁾, E, C; preferably W |
| 104 | G | G, A, S, T, D, P, N, E, C, L; preferably |
| | | G |
| 108 | L, M or T; | Q, L⁽⁷⁾, R, P, E, K, S, T, M, A, H; |
| | predominantly L | preferably Q or L⁽⁷⁾ |

Notes:
In particular, but not exclusively, in combination with KERE or KQRE at positions 43-46.
Usually as GLEW at positions 44-47.
Usually as KERE or KORE at positions 43-46, e.g., as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
With both GLEW at positions 44-47 and KERE or KQRE at positions 43-46.
Often as KP or EP at positions 83-84 of naturally occurring V_{HH} domains.
In particular, but not exclusively, in combination with GLEW at positions 44-47.
With the proviso that when positions 44-47 are GLEW, position 108 is always Q in (non-humanized) V_{HH} sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW

### 5.2 Specificity

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, preferably specifically bind to their designated targets. The specificity/selectivity of a binding unit can be determined based on affinity. The affinity denotes the strength or stability of a molecular interaction. The affinity is commonly given as by the K_{D}, or dissociation constant, which is expressed in units of mol/liter (or M). The affinity can also be expressed as an association constant, K_{A}, which equals 1/K_{D} and is expressed in units of (mol/liter)⁻¹ (or M⁻¹). The affinity is a measure for the binding strength between a moiety and a binding site on the target molecule: the lower the value of the K_{D}, the stronger the binding strength between a target molecule and a targeting moiety. Typically, binding units used in the present technology (such as ISVDs) will bind to their targets with a dissociation constant (K_{D}) of 10⁻⁵ to 10¹² moles/liter or less, and preferably 10⁻⁷ to 10⁻¹² moles/liter or less and more preferably 10⁻⁸ to 10⁻¹ moles/liter (i.e., with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁻⁴ mol/liter (or any K_{A} value lower than 104 liters/mol) is generally considered to indicate non-specific binding. The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 μM) or less. Accordingly, specific/selective binding may mean that—using the same measurement method, e.g., SPR—a binding unit (or polypeptide comprising the same) binds to FcRn with a K_{D} value of 10⁻⁵ to 10⁻¹² moles/liter or less and binds to related targets with a K_{D} value greater than 10⁻⁴ moles/liter. Thus, the ISVD preferably exhibits at least half the binding affinity, more preferably at least the same binding affinity, to human FcRn as compared to an ISVD consisting of the amino acid of SEQ ID NO's: 14 or 15, wherein the binding affinity is measured using the same method, such as SPR. Specific binding to a certain target from a certain species does not exclude that the binding unit can also specifically bind to the analogous target from a different species. For example, specific binding to human FcRn does not exclude that the binding unit (or a polypeptide comprising the same) can also specifically bind to FcRn from cynomolgus monkeys. Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}]. The affinity of a molecular interaction between two molecules can be measured via different techniques known per se, such as the well-known surface plasmon resonance (SPR) biosensor technique (see for example Ober et al., 2001, Intern. Immunology 13: 30 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al. (1991, Anal. Biochem. 198: 268-277). Another well-known biosensor technique to determine affinities of biomolecular interactions is bio-layer interferometry (BLI) (see for example Abdiche et al., 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA). Alternatively, affinities can be measured in Kinetic Exclusion Assay (KinExA) (see for example Drake et al., 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen). The GYROLAB® immunoassay system provides a platform for automated bioanalysis and rapid sample turnaround (Fraley et al., 2013, Bioanalysis 5: 1765-74).

In particular embodiments, the polypeptides according to the invention specifically bind to the above-described epitope on FcRn with an affinity (K_{A}) of between 10⁶ M⁻¹ and 10¹¹ M⁻¹.

In particular embodiments, the polypeptides according to the invention specifically bind to the above-described epitope on FcRn with a dissociation constant (K_{D}) of between 10⁻⁶ nM and 10⁻¹¹ M or less. Preferably, the K_{D} is determined by Kinexa, BLI or SPR, for instance as determined by SPR.

In particular embodiments, the polypeptides according to the invention specifically bind to the above-described epitope on FcRn with an on-rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶ M⁻¹s⁻¹, at least about 10⁷ M⁻¹s⁻¹, and at least about 10⁸ M⁻¹s⁻¹, preferably as measured by surface plasmon resonance or BLI.

In particular embodiments, the polypeptides according to the invention specifically bind to the above-described epitope on FcRn with an off-rate constant (k_{off}) selected from the group consisting of at most about 10¹s⁻¹, at most about 10⁻² s⁻¹, at most about 10⁻³ s⁻¹, of at most about 10⁻⁴ s⁻¹, at most about 10⁻⁵ s⁻¹, and at most about 10⁻⁶ s⁻¹, preferably as measured by surface plasmon resonance or BLI.

The FcRn binding polypeptides of the present invention are, in certain embodiments, such that they are cross-reactive between human FcRn and FcRn from at least one, preferably from at least two, more preferably from at least three and up to essentially all of the following species of mammal: mouse, dog, rat, rabbit, guinea pig, pig, sheep, cow and cynomolgus monkey.

When an ISVD is said to exhibit “(improved) cross-reactivity for binding to human and non-human primate FcRn” compared to another ISVD, it means that for said ISVD the ratio of the binding activity (such as expressed in terms of K_{D} or k_{off}) for human FcRn and for non-human primate FcRn is lower than that same ratio calculated for the other ISVD in the same assay. Good cross-reactivity for binding to human and non-human primate FcRn allows for the assessment of toxicity of an FcRn binding polypeptide according to the present invention in preclinical studies conducted on non-human primates.

In particular embodiments, the FcRn binding polypeptides of the present invention are such that they are (at least) cross-reactive between human FcRn and cynomolgus monkey FcRn, and preferably also between either human FcRn and/or cynomolgus monkey FcRn on the one hand, and at least one, preferably both of rat FcRn and pig FcRn on the other hand. For the sake of convenience, in the sequence of FcRn, the stretches of amino acids that are assumed to be part of the putative epitope of the polypeptides of the invention have been highlighted. Without being limited to any specific mechanism or hypothesis, it is assumed that the polypeptides of the invention are (essentially) capable of binding to (one or more amino acid residues within) the corresponding stretches of amino acid residues that are present within the amino acid sequence of those mammalian FcRn proteins, with which the polypeptides of the invention are cross-reactive.

Generally, an FcRn binding polypeptide of the invention can be considered to be cross-reactive between human FcRn and FcRn from one of the above mentioned other species when it can bind to human FcRn with an affinity less than 500 nM, preferably less than 200 nM, more preferably less than 10 nM; and also to FcRn from those above-mentioned species with an affinity less than 500 nM, preferably less than 200 nM, more preferably less than 10 nM, again both as determined using SPR.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 2 [=X₁ X₂ X₃ M Y] or CDR1 (according to AbM) has the amino acid sequence of SEQ ID NO: 5 [=Xₐ X_{b} X_{c} F Xₑ X₁ X₂ X₃ M Y], wherein
Xₐ is A, D, E, F, G, H, K, L, M, N, Q, S, T, W, or Y,
X_{b} is F, I, L, M, V, or Y,
X_{c} is A, E, G, I, L, M, N, P, S, T, V, W, or Y,
Xₑ is D, E, G, K, M, N, P, Q, S or T,
X₁ is A, D, E, G, K, N, Q, S, T, or V,
X₂ is A, F, L, M, N, Q, S, T, V, or Y, and
X₃ is A, D, E, G, H, M, N, Q, S, T, or V,
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is A, D, E, G, H, P, Q, R, S, T, or V,
X₄ is A, D, E, G, K, M, N, P, Q, R, S, T, V, W, or Y,
X₈ is A, D, G, H, K, L, M, Q, S, T, or V,
X₉ is A, D, E, F, G, H, I, K, L, M, N, P, Q, R, S, T, V, W, or Y, and
X₁₀ is A, D, E, H, S, T, V, or Y,
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7 [=D X₂ X₃ X₄ T X₆ X₇ T X₉ Y X₁₁ X₁₂], wherein
X₂ is A, D, E, F, G, I, L, M, Q, S, T, V, W, or Y, preferably A, E, F, G, I, L, M, Q, S, T, V, W, or Y
X₃ is L, or N, preferably L,
X₄ is F, L, W, or Y,
X₆ is A, D, E, K, L, M, Q, S, or W,
X₇ is L, M, or V,
X₉ is A, D, E, F, G, H, K, L, M, Q, R, S, T, V, W or Y,
X₁₁ is A, E, L, M, Q, S, T, V, or W, and
X¹² is T or Y,
provided that the amino acids
X₁ and X₃ of SEQ ID NO: 2 and X₆ of SEQ ID NO: 4 are not simultaneously E, D, and D, respectively, or
Xₐ, X₁ and X₃ of SEQ ID NO: 5 and X₆ of SEQ ID NO: 7 are not simultaneously T, E, D, and D, respectively, or
X₁ and X₃ of SEQ ID NO: 2 and X₉₀f SEQ ID NO: 4 are not simultaneously E, D, and K, respectively, or
Xₐ, X₁ and X₃ of SEQ ID NO: 5 and X₉ of SEQ ID NO: 7 are not simultaneously T, E, D, and K, respectively, or
X₃ of SEQ ID NO: 2 and X₉ of SEQ ID NO: 4 are not simultaneously D and K, respectively, or
Xₐ and X₃ of SEQ ID NO: 5 and X₉ of SEQ ID NO: 7 are not simultaneously T, D and K, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
Xₐ and X₁ of SEQ ID NO: 5, X₄ of SEQ ID NO: 6, and X₆ and X₉ of SEQ ID NO: 7 are not simultaneously T, E, Q, D, and K, respectively, or
X₃ of SEQ ID NO: 2 and X₆ of SEQ ID NO: 4 are not simultaneously D, and D, respectively, or
Xₐ and X₃ of SEQ ID NO: 5 and X₆₀f SEQ ID NO: 7 are not simultaneously T, D, and D, respectively, or
X₁ and X₃ of SEQ ID NO: 2, X₄ of SEQ ID NO: 3, and X₆ of SEQ ID NO: 4 are not simultaneously E, D, Q, and D, respectively, or
Xₐ, X₁ and X₃ of SEQ ID NO: 5, X₄ of SEQ ID NO: 6, and X₆ of SEQ ID NO: 7 are not simultaneously T, E, D, Q, and D, respectively, or
X₁ of SEQ ID NO: 2, and X₆ and X₉ of SEQ ID NO: 4 are not simultaneously E, D, and K, respectively, or
X₃ of SEQ ID NO: 2, X₄ of SEQ ID NO: 3, and X₆ and X₉ of SEQ ID NO: 4 are not simultaneously D, Q, D, and K, respectively, or
X₁ and X₃ of SEQ ID NO: 2, X₄ of SEQ ID NO: 3, and X₉ of SEQ ID NO: 4 are not simultaneously E, D, Q, and K, respectively, or
Xₐ, X₁ and X₃ of SEQ ID NO: 5, X₄ of SEQ ID NO: 6, and X₉ of SEQ ID NO: 7 are not simultaneously T, E, D, Q, and K, respectively, or
X₃ of SEQ ID NO: 2, and X₆ and X₉ of SEQ ID NO: 4 are not simultaneously D, D, and K, respectively, or
X₃ of SEQ ID NO: 2, and X₉ of SEQ ID NO: 4 are not simultaneously D, and S, respectively, or
Xₐ and X₃ of SEQ ID NO: 5, and X₉ of SEQ ID NO: 7 are not simultaneously T, D, and S, respectively, or
X₁ and X₃ of SEQ ID NO: 2, and X₆ and X₉ of SEQ ID NO: 4 are not simultaneously E, D, D, and S, respectively, or
X₆ and X₉ of SEQ ID NO: 4 are not simultaneously D, and K, respectively, or
Xₐ of SEQ ID NO: 5, and X₆ and X₉ of SEQ ID NO: 7 are not simultaneously T, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
X₃ of SEQ ID NO: 2, X₄ of SEQ ID NO: 3, and X₆ and X₉ of SEQ ID NO: 4 are not simultaneously D, Q, D, and S, respectively, or
Xₐ and X₃ of SEQ ID NO: 5, X₄ of SEQ ID NO: 6, and X₆ and X₉ of SEQ ID NO: 7 are not simultaneously T, D, Q, D, and S, respectively, or
X₁ of SEQ ID NO: 2, X₄ of SEQ ID NO: 3, X₆ and X₉ of SEQ ID NO: 4 are not simultaneously E, Q, D, and S, respectively, or
Xₐ and X₁ of SEQ ID NO: 5, X₄ of SEQ ID NO: 6, X₆ and X₉ of SEQ ID NO: 7 are not simultaneously T, E, Q, D, and S, respectively, or
X₁ of SEQ ID NO: 2, and X₆ and X₉ of SEQ ID NO: 4 are not simultaneously E, D, and K, respectively, or
Xₐ and X₁ of SEQ ID NO: 5, and X₆ and X₉ of SEQ ID NO: 7 are not simultaneously T, E, D, and K, respectively, or
X₃ of SEQ ID NO: 2, and X₆ and X₉ of SEQ ID NO: 4 are not simultaneously D, D, and K, respectively, or
Xₐ and X₃ of SEQ ID NO: 5, and X₆ and X₉ of SEQ ID NO: 7 are not simultaneously T, D, D, and K, respectively, or
X₁ and X₃ of SEQ ID NO: 2, and X₆ and X₉ of SEQ ID NO: 4 are not simultaneously E, D, D, and K, respectively, or
X₃ of SEQ ID NO: 2, and X₆ and X₉ of SEQ ID NO: 4 are not simultaneously D, D, and S, respectively, or,
X₁ and X₃ of SEQ ID NO: 2, X₄ of SEQ ID NO: 3, and X₆ and X₉ of SEQ ID NO: 4 are not simultaneously E, D, Q, D, and S, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and S, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, and X₆ and X₉ of SEQ ID NO: 7 are not simultaneously T, E, D, D, and S, respectively, or
X₃ of SEQ ID NO: 2, X₆ and X₉ of SEQ ID NO: 4 are not simultaneously D, D, and S, respectively, or
Xₐ and X₃ of SEQ ID NO: 5, X₆ and X₉ of SEQ ID NO: 7 are not simultaneously T, D, D, and S, respectively, or
X₁ and X₃ of SEQ ID NO: 2, and X₉ of SEQ ID NO: 4 are not simultaneously E, D, and S, respectively, or
X₄ of SEQ ID NO: 3, and X₉ of SEQ ID NO: 4 are not simultaneously Q, and S, respectively or Xₐ of SEQ ID NO: 5, X₄ of SEQ ID NO: 6, and X₉ of SEQ ID NO: 7 are not simultaneously T, Q, and S, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 2 [=X₁ X₂ X₃ M Y] or CDR1 (according to AbM) has the amino acid sequence of SEQ ID NO: 5 [=Xₐ X_{b} X_{c} F Xₑ X₁ X₂X₃ M Y], wherein
Xₐ is G, or T,
X_{b} is F,
X_{c} is T,
Xₑ is S,
X₁ is S, D, or E,
X₂ is Y, and
X₃ is A, or D,
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is S,
X₄ is Q, or S,
X₈ is S,
X₉ is T, and
X₁₀ is D,
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7 [=D X₂ X₃ X₄ T X₆ X₇ T X₉ Y X₁₁ X₁₂], wherein
X₂ is T,
X₃ is L,
X₄ is Y,
X₆ is D, E, or S,
X₇ is L,
X₉ is K, R, S, or W,
X₁₁ is S, and
X₁₂ is Y,
provided that the amino acids
X₁ and X₃ of SEQ ID NO: 2 and X₆ of SEQ ID NO: 4 are not simultaneously E, D, and D, respectively, or
Xₐ, X₁ and X₃ of SEQ ID NO: 5 and X₆ of SEQ ID NO: 7 are not simultaneously T, E, D, and D, respectively, or
X₁ and X₃ of SEQ ID NO: 2 and X₉₀f SEQ ID NO: 4 are not simultaneously E, D, and K, respectively, or
Xₐ, X₁ and X₃ of SEQ ID NO: 5 and X₉ of SEQ ID NO: 7 are not simultaneously T, E, D, and K, respectively, or
X₃ of SEQ ID NO: 2 and X₉ of SEQ ID NO: 4 are not simultaneously D and K, respectively, or
Xₐ and X₃ of SEQ ID NO: 5 and X₉ of SEQ ID NO: 7 are not simultaneously T, D and K, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
Xₐ and X₁ of SEQ ID NO: 5, X₄ of SEQ ID NO: 6, and X₆ and X₉ of SEQ ID NO: 7 are not simultaneously T, E, Q, D, and K, respectively, or
X₃ of SEQ ID NO: 2 and X₆ of SEQ ID NO: 4 are not simultaneously D, and D, respectively, or
Xₐ and X₃ of SEQ ID NO: 5 and X₆₀f SEQ ID NO: 7 are not simultaneously T, D, and D, respectively, or
X₁ and X₃ of SEQ ID NO: 2, X₄ of SEQ ID NO: 3, and X₆ of SEQ ID NO: 4 are not simultaneously E, D, Q, and D, respectively, or
Xₐ, X₁ and X₃ of SEQ ID NO: 5, X₄ of SEQ ID NO: 6, and X₆ of SEQ ID NO: 7 are not simultaneously T, E, D, Q, and D, respectively, or
X₁ of SEQ ID NO: 2, and X₆ and X₉ of SEQ ID NO: 4 are not simultaneously E, D, and K, respectively, or
X₃ of SEQ ID NO: 2, X₄ of SEQ ID NO: 3, and X₆ and X₉ of SEQ ID NO: 4 are not simultaneously D, Q, D, and K, respectively, or
X₁ and X₃ of SEQ ID NO: 2, X₄ of SEQ ID NO: 3, and X₉ of SEQ ID NO: 4 are not simultaneously E, D, Q, and K, respectively, or
Xₐ, X₁ and X₃ of SEQ ID NO: 5, X₄ of SEQ ID NO: 6, and X₉ of SEQ ID NO: 7 are not simultaneously T, E, D, Q, and K, respectively, or
X₃ of SEQ ID NO: 2, and X₆ and X₉ of SEQ ID NO: 4 are not simultaneously D, D, and K, respectively, or
X₃ of SEQ ID NO: 2, and X₉ of SEQ ID NO: 4 are not simultaneously D, and S, respectively, or
Xₐ and X₃ of SEQ ID NO: 5, and X₉ of SEQ ID NO: 7 are not simultaneously T, D, and S, respectively, or
X₁ and X₃ of SEQ ID NO: 2, and X₆ and X₉ of SEQ ID NO: 4 are not simultaneously E, D, D, and S, respectively, or
X₆ and X₉ of SEQ ID NO: 4 are not simultaneously D, and K, respectively, or
Xₐ of SEQ ID NO: 5, and X₆ and X₉ of SEQ ID NO: 7 are not simultaneously T, D, and K, respectively, or
X₁ of SEQ ID NO: 2, X₄ of SEQ ID NO: 3, and X₆ and X₉ of SEQ ID NO: 4 are not simultaneously E, Q, D, and K, respectively, or
X₃ of SEQ ID NO: 2, X₄ of SEQ ID NO: 3, and X₆ and X₉ of SEQ ID NO: 4 are not simultaneously D, Q, D, and S, respectively, or
Xₐ and X₃ of SEQ ID NO: 5, X₄ of SEQ ID NO: 6, and X₆ and X₉ of SEQ ID NO: 7 are not simultaneously T, D, Q, D, and S, respectively, or
X₁ of SEQ ID NO: 2, X₄ of SEQ ID NO: 3, X₆ and X₉ of SEQ ID NO: 4 are not simultaneously E, Q, D, and S, respectively, or
Xₐ and X₁ of SEQ ID NO: 5, X₄ of SEQ ID NO: 6, X₆ and X₉ of SEQ ID NO: 7 are not simultaneously T, E, Q, D, and S, respectively, or
X₁ of SEQ ID NO: 2, and X₆ and X₉ of SEQ ID NO: 4 are not simultaneously E, D, and K, respectively, or
Xₐ and X₁ of SEQ ID NO: 5, and X₆ and X₉ of SEQ ID NO: 7 are not simultaneously T, E, D, and K, respectively, or
X₃ of SEQ ID NO: 2, and X₆ and X₉ of SEQ ID NO: 4 are not simultaneously D, D, and K, respectively, or
Xₐ and X₃ of SEQ ID NO: 5, and X₆ and X₉ of SEQ ID NO: 7 are not simultaneously T, D, D, and K, respectively, or
X₁ and X₃ of SEQ ID NO: 2, and X₆ and X₉ of SEQ ID NO: 4 are not simultaneously E, D, D, and K, respectively, or
X₃ of SEQ ID NO: 2, and X₆ and X₉ of SEQ ID NO: 4 are not simultaneously D, D, and S, respectively, or,
X₁ and X₃ of SEQ ID NO: 2, X₄ of SEQ ID NO: 3, and X₆ and X₉ of SEQ ID NO: 4 are not simultaneously E, D, Q, D, and S, respectively, or
Xₐ, X₁ and X₃ of SEQ ID NO: 5, X₄ of SEQ ID NO: 6, and X₆ and X₉ of SEQ ID NO: 7 are not simultaneously T, E, D, Q, D, and S, respectively, or
X₁ and X₃ of SEQ ID NO: 2, X₄ of SEQ ID NO: 3, and X₆ and X₉ of SEQ ID NO: 4 are not simultaneously E, D, Q, D, and K, respectively, or
Xₐ, X₁ and X₃ of SEQ ID NO: 5, and X₆ and X₉ of SEQ ID NO: 7 are not simultaneously T, E, D, D, and S, respectively, or
X₃ of SEQ ID NO: 2, X₆ and X₉ of SEQ ID NO: 4 are not simultaneously D, D, and S, respectively, or
Xₐ and X₃ of SEQ ID NO: 5, X₆ and X₉ of SEQ ID NO: 7 are not simultaneously T, D, D, and S, respectively, or X₁ and X₃ of SEQ ID NO: 2, and X₉ of SEQ ID NO: 4 are not simultaneously E, D, and S, respectively, or
X₄ of SEQ ID NO: 3, and X₉ of SEQ ID NO: 4 are not simultaneously Q, and S, respectively or
Xₐ of SEQ ID NO: 5, X₄ of SEQ ID NO: 6, and X₉ of SEQ ID NO: 7 are not simultaneously T, Q, and S, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or CDR1 (according to AbM) has the amino acid sequence of SEQ ID NO: 5 [=Xₐ X_{b} X_{c} F Xₑ X₁ X₂X₃ M Y], wherein
Xₐ is A, D, E, F, G, H, K, L, M, N, Q, S, T, W, or Y,
X_{b} is F, I, L, M, V, or Y,
X_{c} is A, E, G, I, L, M, N, P, S, T, V, W, or Y,
Xₑ is D, E, G, K, M, N, P, Q, S or T,
X₁ is A, D, E, G, K, N, Q, S, T, or V,
X₂ is A, F, L, M, N, Q, S, T, V, or Y, and
X₃ is A, D, E, G, H, M, N, Q, S, T, or V,
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is A, D, E, G, H, P, Q, R, S, T, or V,
X₄ is A, D, E, G, K, M, N, P, Q, R, S, T, V, W, or Y,
X₈ is A, D, G, H, K, L, M, Q, S, T, or V,
X₉ is A, D, E, F, G, H, I, K, L, M, N, P, Q, R, S, T, V, W, or Y, and
X₁₀ is A, D, E, H, S, T, V, or Y,
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7[=D X₂ X₃ X₄ T X₆ X₇ T X₉ Y X₁₁ X₁₂], wherein
X₂ is A, D, E, F, G, I, L, M, Q, S, T, V, W, or Y, preferably A, E, F, G, I, L, M, Q, S, T, V, W, or Y
X₃ is L, or N, preferably L,
X₄ is F, L, W, or Y,
X₆ is A, D, E, K, L, M, Q, S, or W,
X₇ is L, M, or V,
X₉ is A, D, E, F, G, H, K, L, M, Q, R, S, T, V, W or Y,
X₁₁ is A, E, L, M, Q, S, T, V, or W, and
X₁₂ is T or Y,
provided that the amino acids X₆ and X₉ of SEQ ID NO: 4 are not simultaneously D, and K, respectively, or X₆ and X₉ of SEQ ID NO: 7 are not simultaneously D, and K, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 2 [=X₁ X₂ X₃ M Y] or CDR1 (according to AbM) has the amino acid sequence of SEQ ID NO: 5 [=Xₐ X_{b} X_{c} F Xₑ X₁ X₂X₃ M Y], wherein
Xₐ is G, or T,
X₁ is S, D, or E,
X₂ is Y, and
X₃ is A, or D,
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y] and
CDR2 (according to Kabat) has the amino acid sequence of SEQ ID NO: 3 [=A I X₃ X₄ G G G X₈ X₉ X₁₀ Y A D S V K G] or CDR2 (according to AbM) has the amino acid sequence of SEQ ID NO: 6 [=A I X₃ X₄ G G G X₈ X₉ X₁₀], wherein
X₃ is S,
X₄ is Q, or S,
X⁸ is S,
X₉ is T, and
X₁₀ is D,
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D],
and
CDR3 (according to Kabat) has the amino acid sequence of SEQ ID NO: 4 [=D X₂ X₃ X₄ T X₆ X₇ T X₉ Y X₁₁ X₁₂] or CDR3 (according to AbM) has the amino acid sequence of SEQ ID NO: 7[=D X₂ X₃ X₄ T X₆ X₇ T X₉ Y X₁₁ X₁₂], wherein
X₂ is T,
X₃ is L,
X₄ is Y,
X₆ is D, E, or S,
X₇ is L,
X₉ is K, R, S, or W,
X₁₁ is S, and
X₁₂ is Y,
provided that the amino acids X₆ and X₉ of SEQ ID NO: 4 are not simultaneously D, and K, respectively, or X₆ and X₉ of SEQ ID NO: 7 are not simultaneously D, and K, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into A, D, E, G, K, N, Q, T, or V, and/or
Y at position 2 of SEQ ID NO: 8 or at position 7 of SEQ ID NO: 11 has been changed into A, F, L, M, N, Q, S, T, or V, and/or
A at position 3 of SEQ ID NO: 8 or at position 8 of SEQ ID NO: 11 has been changed into D, E, G, H, M, N, Q, S, T, or V and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 3 of SEQ ID NO: 9 or at position 3 of SEQ ID NO: 12 has been changed into A, D, E, G, H, P, Q, R, T, or V, and/or
S at position 4 of SEQ ID NO: 9 or at position 4 of SEQ ID NO: 12 has been changed into A, D, E, G, K, M, N, P, Q, R, T, V, W, or Y and/or
S at position 8 of SEQ ID NO: 9 or at position 8 of SEQ ID NO: 12 has been changed into A, D, G, H, K, L, M, Q, T, or V, and/or
T at position 9 of SEQ ID NO: 9 or at position 9 of SEQ ID NO: 12 has been changed into is A, D, E, F, G, H, I, K, L, M, N, P, Q, R, S, V, W, or Y and/or
D at position 10 of SEQ ID NO: 9 or at position 10 of SEQ ID NO: 12 has been changed into A, E, H, S, T, V, or Y, and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y]
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D]
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y], wherein
T at position 2 of SEQ ID NO: 10 or at position 2 of SEQ ID NO: 13 has been changed into A, D, E, F, G, I, L, M, Q, S, V, W, or Y, preferably into A, E, F, G, I, L, M, Q, S, V, W, or Y, and/or
L at position 3 of SEQ ID NO: 10 or at position 3 of SEQ ID NO: 13 has been changed into N, preferably wherein the amino acid at position 3 of SEQ ID NO: 13 is L (i.e., it has not been changed), and/or
Y at position 4 of SEQ ID NO: 10 or at position 4 of SEQ ID NO: 13 has been changed into F, L, or W, and/or
S at position 6 of SEQ ID NO: 10 or at position 6 of SEQ ID NO: 13 has been changed into A, D, E, K, L, M, Q, or W, and/or
L at position 7 of SEQ ID NO: 10 or at position 7 of SEQ ID NO: 13 has been changed into M, or V, and/or
S at position 9 of SEQ ID NO: 10 or at position 9 of SEQ ID NO: 13 has been changed into A, D, E, F, G, H, K, L, M, Q, R, T, V, W or Y, and/or
S at position 11 of SEQ ID NO: 10 or at position 11 of SEQ ID NO: 13 has been changed into A, E, L, M, Q, T, V, or W, and/or
Y at position 12 of SEQ ID NO: 10 or at position 12 of SEQ ID NO: 13 has been changed into T, provided that the amino acids at positions 6 and 9 of SEQ ID NO: 10 or at positions 6 and 9 of SEQ ID NO: 13 are not simultaneously changed into D, and K, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that:
CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into D or E, and/or
A at position 3 of SEQ ID NO: 8 or at position 8 of SEQ ID NO: 11 has been changed into D,
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 4 of SEQ ID NO: 9 or at position 4 of SEQ ID NO: 12 has been changed into Q,
and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y], wherein
S at position 6 of SEQ ID NO: 10 or at position 6 of SEQ ID NO: 13 has been changed into D, or E, and/or
S at position 9 of SEQ ID NO: 10 or at position 9 of SEQ ID NO: 13 has been changed into K, R, or W, provided that the amino acids at positions 6 and 9 of SEQ ID NO: 10 or at positions 6 and 9 of SEQ ID NO: 13 are not simultaneously changed into D, and K, respectively.

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], wherein
S at position 1 of SEQ ID NO: 8 or at position 6 of SEQ ID NO: 11 has been changed into D or E, and/or
A at position 3 of SEQ ID NO: 8 or at position 8 of SEQ ID NO: 11 has been changed into D, and CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y], and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], wherein
S at position 4 of SEQ ID NO: 9 or at position 4 of SEQ ID NO: 12 has been changed into Q, and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y].

In particular embodiments, the present invention provides FcRn binding polypeptides comprising at least one ISVD specifically binding to the epitope on FcRn as disclosed herein, wherein at least one ISVD consists of 4 framework regions (FR1 to FR4 respectively) and 3 complementarity determining regions (CDR1 to CDR3 respectively) and is characterized in that: CDR1 (according to Kabat) has the amino acid sequence of SEQ ID NO: 8 [=S Y A M Y] or CDR1 (according to AbM) has the amino acid sequence of SEQ ID NO: 11 [=G F T F S S Y A M Y],
and
CDR2 (according to Kabat) has the amino acid sequence of SEQ ID NO: 9 [=A I S S G G G S T D Y A D S V K G] or CDR2 (according to AbM) has the amino acid sequence of SEQ ID NO: 12 [=A I S S G G G S T D], and
CDR3 (according to Kabat) has the amino acid sequence of SEQ ID NO: 10 [=D T L Y T S L T S Y S Y] or
CDR3 (according to AbM) has the amino acid sequence of SEQ ID NO: 13 [=D T L Y T S L T S Y S Y], wherein
S at position 6 of SEQ ID NO: 10 or at position 6 of SEQ ID NO: 13 has been changed into D, or E, and/or
S at position 9 of SEQ ID NO: 10 or at position 9 of SEQ ID NO: 13 has been changed into K, R, or W, provided that the amino acids at positions 6 and 9 of SEQ ID NO: 10 or at positions 6 and 9 of SEQ ID NO: 13 are not simultaneously changed into D, and K, respectively.

In certain particular embodiments, the present invention provides polypeptides characterized in that the at least one ISVD binding specifically to FcRn consists of 4 framework regions (FR1 to FR4 respectively) and 3 complementarity determining regions (CDR1 to CDR3 respectively), and is characterized in that:
a) CDR1 (according to Kabat) has the amino acid sequence of SEQ ID NO: 8 [=S Y A M Y] and/or has an amino acid sequence having 4, 3, 2 or only 1 “amino acid difference(s)” (as defined herein) with the sequence of SEQ ID NO: 8; and
b) CDR2 (according to Kabat) has the amino acid sequence of SEQ ID NO: 9 [=A I S S G G G S T D Y A D S V K G] and/or has an amino acid sequence having 4, 3, 2 or only 1 “amino acid difference(s)” (as defined herein) with the sequence of SEQ ID NO: 9; and
c) CDR3 (according to Kabat) has the amino acid sequence of SEQ ID NO: 10 [=D T L Y T S L T S Y S Y] and/or has an amino acid sequence having 4, 3, 2 or only 1 “amino acid difference(s)” (as defined herein) with the sequence of SEQ ID NO: 10.

In certain particular embodiments, the present invention provides polypeptides characterized in that the at least one ISVD binding specifically to FcRn consists of 4 framework regions (FR1 to FR4 respectively) and 3 complementarity determining regions (CDR1 to CDR3 respectively), and is characterized in that:
a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13.

In particular embodiments, the FcRn binding polypeptides of the present invention are characterized in that the at least one ISVD specifically binding to the epitope on FcRn as disclosed herein has the sequence of SEQ ID NO: 14 or SEQ ID NO: 15.

In particular embodiments, the FcRn binding polypeptides of the present invention are characterized in that the at least one ISVD specifically binding to the epitope on FcRn as disclosed herein has at least 80%, such as 90%, or even 95% sequence identity with the amino acid sequence of SEQ ID NO: 14 or SEQ ID NO: 15.

In certain particular embodiments, the present invention provides polypeptides as described herein characterized in that the ISVD is a (single) domain antibody, a Nanobody® V_{HH}, a V_{HH}, a humanized V_{HH}, or a camelized V_{H}.

In particular embodiments, the polypeptides as described herein comprising the ISVD with the one or more CDRs with 1, 2, 3, or 4 amino acid(s) differences bind to FcRn with about the same affinity compared to the binding by the amino acid sequence or polypeptide comprising the CDRs without the 4, 3, 2, or 1 amino acid(s) difference, said affinity as measured by surface plasmon resonance.

When comparing two immunoglobulin single variable domains, the term “amino acid difference” refers to an insertion, deletion, or substitution of a single amino acid residue on a position of the first sequence, compared to the second sequence; it being understood that two immunoglobulin single variable domains can contain one, two or more such amino acid differences.

For the purposes of comparing two or more immunoglobulin single variable domains or other amino acid sequences such e.g. the polypeptides of the invention etc., the percentage of “sequence identity” between a first amino acid sequence and a second amino acid sequence (also referred to herein as “amino acid identity”) may be calculated or determined as described in paragraph f) on pages 49 and 50 of WO 08/020079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein; alternatively, the degree of sequence identity between two amino acid sequences may be calculated using a known computer algorithm for sequence alignment, such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two immunoglobulin single variable domains, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Examples of conservative substitutions are described herein further below.

Any amino acid substitutions applied to the polypeptides described herein may also be based on the analysis of the frequencies of amino acid variations between homologous proteins of different species developed by Schulz et al, 1978 (Principles of Protein Structure, Springer-Verlag), on the analyses of structure forming potentials developed by Chou and Fasman 1975 (Biochemistry 13: 211) and 1978 (Adv. Enzymol. 47: 45-149), and on the analysis of hydrophobicity patterns in proteins developed by Eisenberg et al., 1984 (Proc. Natl. Acad. Sci. USA 81: 140-144), Kyte & Doolittle 1981 (J Molec. Biol. 157: 105-132), and Goldman et al., 1986 (Ann. Rev. Biophys. Chem. 15: 321-353), all incorporated herein in their entirety by reference. Information on the primary, secondary and tertiary structure of ISVDs is given in the description herein and in the general background art cited above. Also, for this purpose, the crystal structure of a V_{HH} domain from a llama is for example given by Desmyter et al. 1996 (Nature Structural Biology, 3: 803), Spinelli et al. 1996 (Natural Structural Biology 3: 752-757), and Decanniere et al., 1999 (Structure, 7: 361). Further information about some of the amino acid residues that in conventional V_{H} domains form the V_{H}/V_{L} interface and potential camelizing substitutions on these positions can be found in the prior art cited above. Immunoglobulin single variable domains and nucleic acid sequences are said to be “exactly the same” if they have 100% sequence identity (as defined herein) over their entire length.

Compared to the sequence of SEQ ID NO: 14 and/or 15, the FcRn binding polypeptides of the invention preferably also contain (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733.

Amino acid sequence modifications of the polypeptides or ISVDs described herein are contemplated. For example, it may be desirable to improve the binding affinity and/or other biological properties of the polypeptides or ISVDs. Amino acid sequence variants of the polypeptides or ISVOs as described herein are prepared by introducing appropriate nucleotide changes into the nucleic acid encoding the polypeptides or ISVOs, or by peptide synthesis.

Such modifications include, for example, deletions from, and/or insertions into, and/or substitutions of, residues within the polypeptides or ISVDs as described herein. Any combination of deletion, insertion, and substitution is made to arrive at the final construct, provided that the final construct possesses the desired characteristics. The amino acid changes also may alter post-translational processes of the binding molecules, such as changing the number or position of glycosylation sites. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs). The substitutions are preferably conservative substitutions as described herein. Additionally or alternatively, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs.

A useful method for identification of certain residues or regions of polypeptides or ISVDs as described herein, that are preferred locations for mutagenesis is called “alanine scanning mutagenesis” as described by Cunningham and Wells 1989 (Science 244: 1081-1085). Here, a residue or group of target residues within the binding molecule is/are identified (e.g., charged residues such as Arg, Asp, His, Lys, and Glu) and replaced by a neutral or negatively charged amino acid (most preferably alanine or polyalanine) to affect the interaction of the amino acids with the epitope. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se needs not to be predetermined. For example, to analyze the performance of a mutation at a given site, ala scanning or random mutagenesis is conducted at a target codon or region and the expressed binding molecule variants are screened for the desired activity.

Preferably, amino acid sequence insertions include amino- and/or carboxyl-terminal fusions ranging in length from 1, 2, 3, 4, 5, 6, 7, 8, 9 or 10 residues to polypeptides containing a hundred or more residues.

Another type of variant is an amino acid substitution variant. These variants have preferably (at least) 1, 2, 3, 4, 5, 6, 7, 3, 9 or 10 amino acid residues in the amino acid sequences, ISVDs or polypeptides replaced by a different residue. The sites of greatest interest for substitution mutagenesis include the CDRs, in particular the hypervariable regions, but FR alterations are also contemplated. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs, it is preferred that the then-obtained “substituted” sequence is at least 60%, more preferably 65%, even more preferably 70%, particularly preferably 75%, more particularly preferably 80% or even more than 90% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the amino acid sequences, ISVDs or polypeptides may have different degrees of identity to their substituted sequences, e.g., CDR1 may have 80%, while CDR3 may have 90%.

Preferred amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, lie, Val and Cys; and (e) aromatic residues: Phe, Tyr and Trp. Further preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu. However, any substitution (including non-conservative substitution) is envisaged as long as the polypeptide retains its capability to specifically bind to the epitope on FcRn as described herein, specifically bind to FcRn at acidic pH (such as pH 5.0 to 6.8) with an affinity as described herein (e.g. with a K_{D} between 10⁻⁶ and 10⁻¹¹ M) and/or its CDRs have an identity of at least 60%, more preferably 65%, even more preferably 70%, particularly preferably 75%, more particularly preferably 80% to the “original” CDR sequence.

As set out in the Example section, only after rigorous screening and selection methods, the present inventors were able to identify ISVDs binding to FcRn in a pH-dependent fashion. Accordingly, the present invention relates to polypeptides comprising at least one ISVD binding to FcRn at an acidic pH, chosen from the group consisting of SEQ ID NOs: 14 and 15 (Table A-1).

Preferred CDR sequences are depicted as SEQ ID NO's 8 to 13 and 104 to 105 in Table A-1. Sequence analysis further revealed that there are a number of possible sequence variations in the CDRs (see SEQ ID NO's 2 to 7 in Table A-1).

TABLE A-1
Sequence IDs of preferred ISVDs and CDR sequences

| | SEQ ID | |
|---|---|---|
| Nanobody ® ISVD ID | NO. | Sequence |
| T0263018B11-parent | 14 | EVQLVESGGGVVQPGGSLRLSCAASGFTFSSYAMYWVRQAP |
| | | GKGLEWVSAISSGGGSTDYADSVKGRFTISRDNSKNTLYLQM |
| | | NSLRPEDTALYYCAADTLYTSLTWYSYWGQGTLVTVSS |
| T0263018B11-W100cS | 15 | EVQLVESGGGVVQPGGSLRLSCAASGFTFSSYAMYWVRQAP |
| | | GKGLEWVSAISSGGGSTDYADSVKGRFTISRDNSKNTLYLQM |
| | | NSLRPEDTALYYCAADTLYTSLTSYSYWGQGTLVTVSS |
| T0263018B11-parent/W100cS | 11 | GFTFSSYAMY |
| (CDR1-AbM) | | |
| T0263018B11-parent/W100cS | 12 | AISSGGGSTD |
| (CDR2-AbM) | | |
| T0263018B11-parent (CDR3- | 105 | DTLYTSLTWYSY |
| AbM) | | |
| T0263018B11-W100cS (CDR3- | 13 | DTLYTSLTSYSY |
| AbM) | | |
| T0263018B11-parent/W100cS | 8 | SYAMY |
| (CDR1-Kabat) | | |
| T0263018B11-parent/W100cS | 9 | AISSGGGSTDYADSVKG |
| (CDR2-Kabat) | | |
| T0263018B11-parent (CDR3- | 104 | DTLYTSLTWYSY |
| Kabat) | | |
| T0263018B11-W100cS (CDR3- | 10 | DTLYTSLTSYSY |
| Kabat) | | |
| CDR1 (variable sequence-Kabat) | 2 | X₁ X₂ X₃ M Y, wherein |
| | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable sequence-Kabat) | 3 | A I X₃ X₄ G G G X₈ X₉ X₁₀ Y ADS V KG, wherein |
| | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, P, Q, R, S, T, V, |
| | | W, or Y, and |
| | | X₁₀ is A, D, E, H, S, T, V, or Y, |
| CDR3 (variable sequence -Kabat) | 4 | D X₂ X₃ X₄ T X₆ X₇ T X₉ Y X₁₁ X₁₂, wherein |
| | | X₂ is A, D, E, F, G, I, L, M, Q, S, T, V, W, or Y, |
| | | preferably X₂ is A, E, F, G, I, L, M, Q, S, T, V, W, |
| | | or Y, |
| | | X₃ is L, or N, preferably X₃ is L, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |
| CDR1 (variable sequence-AbM) | 5 | Xₐ X_{b} X_{c} F Xₑ X₁ X₂ X₃ M Y, wherein |
| | | Xₐ is A, D, E, F, G, H, K, L, M, N, Q, S, T, W, or Y, |
| | | X_{b} is F, I, L, M, V, or Y, |
| | | X_{c} is A, E, G, I, L, M, N, P, S, T, V, W, or Y, |
| | | Xₑ is D, E, G, K, M, N, P, Q, S or T, |
| | | X₁ is A, D, E, G, K, N, Q, S, T, or V, |
| | | X₂ is A, F, L, M, N, Q, S, T, V, or Y, and |
| | | X₃ is A, D, E, G, H, M, N, Q, S, T, or V |
| CDR2 (variable sequence-AbM) | 6 | A I X₃ X₄ G G G X₈ X₉ X₁₀, wherein |
| | | X₃ is A, D, E, G, H, P, Q, R, S, T, or V, |
| | | X₄ is A, D, E, G, K, M, N, P, Q, R, S, T, V, W, or Y, |
| | | X₈ is A, D, G, H, K, L, M, Q, S, T, or V, |
| | | X₉ is A, D, E, F, G, H, I, K, L, M, N, P, Q, R, S, T, V, |
| | | W, or Y, and |
| | | X₁₀ is A, D, E, H, S, T, V, or Y |
| CDR3 (variable sequence-AbM) | 7 | D X₂ X₃ X₄ T X₆ X₇ T X₉ Y X₁₁ X₁₂, wherein |
| | | X₂ is A, D, E, F, G, I, L, M, Q, S, T, V, W, or Y, |
| | | preferably X₂ is A, E, F, G, I, L, M, Q, S, T, V, W, |
| | | or Y, |
| | | X₃ is L, or N, preferably X₃ is L, |
| | | X₄ is F, L, W, or Y, |
| | | X₆ is A, D, E, K, L, M, Q, S, or W, |
| | | X₇ is L, M, or V, |
| | | X₉ is A, D, E, F, G, H, K, L, M, Q, R, S, T, V, W or Y, |
| | | X₁₁ is A, E, L, M, Q, S, T, V, or W, and |
| | | X₁₂ is T or Y |

The polypeptides of the present invention bind to a specific unique and novel epitope on FcRn, which is an epitope that is different from the epitopes on FcRn bound by the natural ligands of FcRn, i.e., serum albumin and IgG (as shown in FIGS. 2A-21D and 3A-3D).

In particular embodiments, the FcRn binding polypeptides according to the present invention are such that when these are bound to or otherwise associated with an FcRn molecule, the binding by the FcRn molecule to serum albumin and/or IgG is not (significantly) affected, reduced or inhibited.

In further particular embodiments, the present invention provides polypeptides characterized in that they comprise at least one ISVD, which specifically binds to amino acid residues on FcRn that are not involved in binding of FcRn to serum albumin and/or to IgG. The involvement of an amino acid residue on FcRn in binding to at least one ISVD, to serum albumin and/or to IgG can be determined, for example, by determining the interaction of the amino acid residue on FcRn with an amino acid residue on the at least one ISVD, with an amino acid residue on serum albumin and/or with an amino acid residue on IgG, e.g. by crystallographic studies. In this particular embodiment, the polypeptides comprise at least one ISVD which specifically binds to an epitope on FcRn that comprises amino acid residues that are not comprised in the epitope on FcRn bound by serum albumin and/or in the epitope on FcRn bound by IgG. In this particular embodiment, the polypeptides comprise at least one ISVD which specifically binds to amino acid residues on FcRn that are not bound by serum albumin and/or by IgG.

According to particular embodiments, the FcRn binding polypeptides of the invention are preferably also such that they compete with the polypeptide with the amino acid sequence of SEQ ID NO's: 14 and/or 15 for binding to FcRn and/or that they “cross-block” (as defined herein) the binding of the polypeptide with the amino acid sequence of SEQ ID NO's: 14 and/or 15 to FcRn.

The terms “cross-block”, “cross-blocked” and “cross-blocking” are used interchangeably herein to mean the ability of an immunoglobulin single variable domain or polypeptide to interfere with the binding of a ligand to its target, such as a natural ligand to its receptor(s). The extent to which an immunoglobulin single variable domain or polypeptide of the invention is able to interfere with the binding of another compound such as the natural ligand to its target and therefore whether it can be said to cross-block according to the invention, can be determined using competition binding assays. One particularly suitable quantitative cross-blocking assay uses a FACS- or an ELISA-based approach or Alphascreen to measure competition between the labelled (e.g., His tagged or biotinylated) immunoglobulin single variable domain or polypeptide according to the invention and the other binding agent in terms of their binding to the target. Suitable FACS-, ELISA- or Alphascreen-displacement-based assays for determining whether a binding molecule cross-blocks or is capable of cross-blocking a polypeptide are well known. It will be appreciated that these assays can be used with any of the immunoglobulin single variable domains or other binding agents described herein. Thus, in general, a cross-blocking polypeptide according to the invention is for example one which will bind to the target in the above cross-blocking assay such that, during the assay and in the presence of a second polypeptide or in the presence of the natural ligand, the recorded displacement of the immunoglobulin single variable domain or polypeptide according to the invention is between 60% and 100% (e.g., in ELISA/Alphascreen based competition assay) or between 80% to 100% (e.g., in FACS based competition assay) of the maximum theoretical displacement (e.g. displacement by cold (e.g., unlabeled) immunoglobulin single variable domain or polypeptide that needs to be cross-blocked) by the to be tested potentially cross-blocking agent that is present in an amount of 0.01 mM or less.

In particular embodiments, the FcRn binding polypeptides of the invention are such that they bind to essentially the same amino acid residues and/or epitope on FcRn as the amino acid residues and/or epitope bound by the polypeptide with the amino acid sequence of SEQ ID NO's: 14 and/or 15, and even more preferably such that they share essentially the same amino acid interactions as the polypeptide with the amino acid sequence of SEQ ID NO's: 14 and/or 15. For this purpose, according to a specific but non-limiting aspect, FcRn binding polypeptides according to the present invention preferably either have the same CDRs as the sequence of SEQ ID NO's: 14 and/or 15, or compared to the sequence of SEQ ID NO's: 14 and/or 15 preferably contain within their CDRs only such mutations (such as conservative amino acid substitutions) that still allow them to undergo the same or essentially the same amino acid interactions with FcRn as the polypeptide with the sequence of SEQ ID NO's: 14 and/or 15.

In particular embodiments, the FcRn binding polypeptides of the present invention specifically bind to the unique epitope on FcRn as disclosed herein in a pH-dependent manner, such that their binding affinity at an acidic pH, in particular at an acidic pH of between 5.0 and 6.8, is at least three times higher than the binding affinity at a neutral or physiological pH of 7.4. In these particular embodiments, the polypeptides of the present invention show conditional specific binding to FcRn and as a result, by making use of the in vivo FcRn-mediated recycling mechanism, have a prolonged in vivo serum half-life. The polypeptides of the present invention can therefore be used to extend the in vivo half-life of therapeutic targets or therapeutic molecules of interest to which they are suitably linked, bound or fused.

A pH of between 5.0 and 6.8 as used herein means an acid physiological pH, for example, it could be a pH value that is less or more than either 5.0 or 6.8 and/or, it could be any pH value in between the range of 5.0 and 6.8, such as a pH value of 5.1, 5.2, 5.3, 5.4, 5.5, 5.6, 5.7, 5.8, 5.9, 6.0, 6.1, 6.2, 6.3, 6.4, 6.5, 6.6, or 6.7 or a bit more or a bit less. A pH of 7.4 as used herein means a neutral physiological pH, for example it could be a pH between 7.2 and 7.4 (and possibly a bit more or a bit less).

The pH-dependent interaction of the FcRn binding polypeptides with FcRn in certain particular embodiments of the present invention ensures that these polypeptides are optimally and rapidly captured in the acidic environment of the endosomes and efficiently released after recycling when encountering pH 7.4 again (i.e., outside the endosomes).

In this way, the present inventors have been able to demonstrate that the polypeptides according to a certain aspect of the present invention, by specifically binding to FcRn in a pH-dependent manner, show an increased half-life in vivo (as demonstrated by the Examples described further herein).

The half-life of a polypeptide according to the invention, or a fusion protein, a construct or a compound comprising the same (and further described herein) can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. In particular, half-life may be as defined in WO 2009/068627.

The in vivo half-life of a polypeptide according to the invention, or a fusion protein, a construct or a compound comprising the same (as further described herein) can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomologus monkeys (Macaca fascicularis) and/or rhesus monkeys (Macaca mulatta) and baboon (Papio ursinus)) a suitable dose of the amino acid sequence, compound or polypeptide of the invention; collecting blood samples or other samples from said animal; determining the level or concentration of the amino acid sequence, compound or polypeptide of the invention in said blood sample; and calculating, from (a plot of) the data thus obtained, the time until the level or concentration of the amino acid sequence, compound or polypeptide of the invention has been reduced by 50% compared to the initial level upon dosing. Reference is for example made to the Experimental Part below, as well as to the standard handbooks, such as Kenneth, A et a, Chemical Stability of Pharmaceuticals: A Handbook for Pharmacists and Peters et a., Pharmacokinete analysis: A Practical Approach (1996). Reference is also made to “Pharmacokinetics”, M Gibaldi & D Perron, published by Marcel Dekker, 2nd Rev. edition (1982). The half-life can be expressed using parameters such as the t½-alpha, t½-beta and the area under the curve (AUC). Reference is for example made to the Experimental Part below, as well as to the standard handbooks, such as Kenneth et a., 1996 (Chemical Stability of Pharmaceuticals: A Handbook for Pharmacists) and Peters et a., 1996 (Pharmacokinetic Analysis: A Practical Approach). Reference is also made to Gibaldi & Perron 1982 (Pharmacokinetics, Dekker M, 2nd Rev. edition). In the present specification, an “increase in half-life” refers to an increase in any one of these parameters, such as any two of these parameters, or essentially all three these parameters. The terms “increase in half-life” or “increased half-life” in particular refer to an increase in the t½-beta, either with or without an increase in the t½-alpha and/or the AUC or both.

Thus, in particular embodiments, the polypeptides according to the invention and fusion proteins, constructs and compounds comprising the same (as further described herein) that comprise at least one ISVD binding to the unique epitope on FcRn according to the invention and as disclosed herein, will have an increased half-life, compared to a known FcRn binding polypeptide, (fusion) protein, construct or compound as described in the prior art.

Generally, the polypeptides according to the invention and fusion proteins, constructs and compounds comprising the same (as further described herein) preferably have a half-life that is at least 1.5 times, preferably at least 2 times, such as at least 5 times, for example at least 10 times or more than 20 times, or greater than the half-life of the known FcRn binding amino acid sequence and protein of the prior art (as measured in either in man or a suitable animal, such as mouse or cynomolgus monkey).

Also, the polypeptides according to the invention and fusion proteins, constructs and compounds comprising the same (as further described herein) preferably have a half-life that is increased with at least 30%, at least 50%, at least 75%, for example at least 100%, or increased with more than 200%, such as more than 300%, more than 400%, more than 500% or greater compared to the half-life of the known FcRn binding amino acid sequence and protein of the prior art (as measured in either in man or a suitable animal, such as mouse or cynomolgus monkey).

Also, the polypeptides according to the invention and fusion proteins, constructs and compounds comprising the same (as further described herein) that comprise at least one ISVD binding to the unique epitope on FcRn and that are fused to another moiety, such as a therapeutic moiety or moieties, will have an increased half-life, compared to the other moiety, such as the therapeutic moiety or moieties per se.

Generally, the constructs or fusion proteins described herein preferably have a half-life that is at least 1.5 times, preferably at least 2 times, such as at least 5 times, for example at least 10 times or more than 20 times, greater than the half-life of the corresponding other moiety, such as a therapeutic moiety per se (as measured in either in man or a suitable animal, such as mouse or cynomolgus monkey).

As mentioned, in one aspect, an FcRn binder of the invention can be used to increase the half-life of (one or more) immunoglobulin single variable domains, such as domain antibodies, single domain antibodies, “dAb's”, V_{HH}s or Nanobody® V_{HH}s (such as V_{HH}s, humanized V_{HH}s or camelized V_{H}s such as camelized human V_{H}s).

Also, FcRn binding polypeptides provided by the invention and fusion proteins, constructs and compounds comprising the same (as further described herein) preferably have a half-life (defined as t½ beta) in man that is more than 1 hour, preferably more than 2 hours, more preferably of more than 6 hours, such as of more than 12 hours, and for example of about one day, two days, one week, two weeks and up to the half-life of serum albumin or IgG in man (estimated to be around 19 days), although the latter may be less critical.

The FcRn binding polypeptides according to the different embodiments of the invention are preferably also such that either:
they have a serum half-life in man (expressed as t½ beta) that is more than 6 hours, preferably more than 12 hours, more preferably of more than 24 hours, even more preferably more than 72 hours; for example of about one week, two weeks and up to the half-life of serum albumin or IgG in man (estimated to be around 19 days); and/or such that: when they are linked to a therapeutic moiety or entity, they confer to the resulting polypeptide of the invention a serum half-life in man (expressed as t½ beta) that is more than 6 hours, preferably more than 12 hours, more preferably of more than 24 hours, even more preferably more than 72 hours; for example of about one week, two weeks and up to the half-life of serum albumin or IgG in man (estimated to be around 19 days).

The half-life in mammalian species other than man will, among other factors, mainly depend on the binding properties (such as affinity) of the FcRn binding polypeptide of the invention for FcRn from said mammalian species as well on the half-life of the naïve serum albumin in said species. According to a preferred embodiment of the invention, when an FcRn binding polypeptide of the invention is cross-reactive (as defined herein) between human FcRn and FcRn from another mammalian species, then the half-life of the FcRn binding polypeptide of the invention (and/or of a compound of the invention comprising said FcRn binding polypeptide) as determined in said species is preferably at least 5%, such as at least 10%, more preferably at least 25%, for example about 50%, and possibly up to 100% of the half-life of serum albumin in said species.

In particular embodiments, at an acidic pH of between 5.0 and 6.8, the polypeptides according to the invention specifically bind to FcRn with an affinity (K_{A}) of between 10⁻³ nM⁻¹ and 10² nM⁻¹. The affinity (K_{A}) of these polypeptides for FcRn at acidic pH, preferably at a pH of between 5.0 and 6.8 is at least three times higher than the affinity (K_{A}) of the same polypeptides for FcRn at neutral or physiologic pH of 7.4.

In further particular embodiments, at an acidic pH of between 5.0 and 6.8, the polypeptides according to the invention bind to FcRn with an affinity that is at least ten times higher than the affinity (K_{A}) for FcRn of the same polypeptides at neutral or physiologic pH of 7.4. In yet further particular embodiments, at an acidic pH of between 5.0 and 6.8, the polypeptides according to the invention bind to FcRn with an affinity that is at least fifty times higher, such as at least hundred times higher than the affinity (K_{A}) for FcRn of the same polypeptides at neutral or physiologic pH of 7.4.

In certain particular embodiments, the present invention provides polypeptides as described herein characterized in that the at least one ISVD binds to FcRn at neutral or physiologic pH of 7.4 with an affinity that is at least three times, such as at least ten times, such as at least fifty times, such as at least hundred times lower than the affinity with which the at least one ISVD binds to FcRn at acidic pH of between 5.0 and 6.8.

In a particular embodiment, at physiologic pH such as at a pH of 7.4, the at least one ISVD binds to FcRn with a K_{A} value lower than 104 liters/mol.

In certain particular embodiments, the present invention provides polypeptides as described herein characterized in that the at least one ISVD does not detectably, selectively or specifically bind to FcRn, or exhibits no or essentially no binding to FcRn at neutral or physiologic pH, such as at a pH of 7.4.

In particular embodiments, at an acidic pH of between 5.0 and 6.8, the polypeptides according to the invention specifically bind to FcRn with a dissociation constant (K_{D}) of between 10⁻⁶ M and 10⁻¹¹ M or less. Preferably, the K_{D} is determined by Kinexa, BLI or SPR, for instance as determined by SPR.

In particular embodiments, at an acidic pH of between 5.0 and 6.8, the polypeptides according to the invention specifically bind to FcRn with a dissociation constant (K_{D}) of between 103 nM and 10² nM or less. The dissociation constant (K_{D}) of these amino acid sequences and polypeptides for FcRn at acidic pH, preferably at a pH of between 5.0 and 6.8 is at least three times better (i.e. lower value) than the dissociation constant (K_{D}) of the same amino acid sequences and polypeptides for FcRn at neutral or physiologic pH of about 7.4. In further particular embodiments, at an acidic pH of between 5.0 and 6.8, the polypeptides according to the invention bind to FcRn with an affinity that is at least ten times higher/better than the affinity (K_{D}) for FcRn of the same polypeptides at neutral or physiologic pH of about 7.4. In yet further particular embodiments, at an acidic pH of between 5.0 and 6.8, the polypeptides according to the invention bind to FcRn with an affinity that is at least fifty times higher, such as at least hundred times higher/better than the affinity (K_{D}) for FcRn of the same polypeptides at neutral or physiologic pH of about 7.4.

In certain particular embodiments, the present invention provides polypeptides as described herein characterized in that the at least one ISVD binds to FcRn at neutral or physiologic pH of 7.4 with an affinity that is at least three times, such as at least ten times, such as at least fifty times, such as at least hundred times lower/worse than the affinity with which the at least one ISVD binds to FcRn at acidic pH of between 5.0 and 6.8.

In a particular embodiment, at physiologic pH such as at a pH of 7.4, the at least one ISVD binds to FcRn with a K_{D} value greater than 10⁻⁴ mol/liter.

In certain particular embodiments, the present invention provides polypeptides as described herein characterized in that the at least one ISVD does not detectably, selectively or specifically bind to FcRn, or exhibits no or essentially no binding to FcRn at neutral or physiologic pH, such as at a pH of 7.4.

Accordingly, the present invention relates to polypeptides as described herein, comprising at least one ISVD that binds to FcRn with an average K_{D} value of between 1000 nM and 10 pM, such as at an average K_{D} value of 900 nM or less, even more preferably at an average K_{D} value of 800 nM or less, such as less than 700, 600, 500, 400, 300, 200, 100, 50 nM or even less, such as less than 40, 30, 20, 10, 5, 1 nM such as less than 500, 400, 300, 200, 100, 90, 80, 70, 60, 50, 40, 30, 20 pM, or even less, such as less than 10 pM. Preferably, the K_{D} is determined by Kinexa, BLI or surface plasmon resonance (SPR), for instance as determined by SPR. Preferably, the average K_{D} is measured by SPR on recombinant protein.

The present invention also relates to polypeptides as described herein, comprising at least one ISVD binding to FcRn at acidic pH with an EC50 value of between 1000 nM and 1 pM, such as at an average EC50 value of 1000 nM or less, even more preferably at an average EC50 value of 900 nM or less, such as less than 800, 700, 600, 500, 400, 300, 200, 100, 50 nM or even less, such as less than 40, 30, 20, 10, 5 or 1 nM or even less, such as less than 500, 400, 300, 200, 100, 90, 80, 70, 60, 50, 40, 30, 20, 10, 5 pM, or even less, such as less than 4 pM.

In particular embodiments, at an acidic pH of between 5.0 and 6.8, the polypeptides according to the invention specifically bind to FcRn with an on rate constant (kₒₙ) selected from the group consisting of at least about 10² M⁻¹s⁻¹, of at least about 10³ M⁻¹s⁻¹, at least about 10⁴ M⁻¹s⁻¹, at least about 10⁵ M⁻¹s⁻¹, at least about 10⁶ M⁻¹s⁻¹, at least about 10⁷ M⁻¹s⁻¹, and at least about 10⁸ M⁻¹s⁻¹, preferably as measured by surface plasmon resonance or BLI.

In particular embodiments, at an acidic pH of between 5.0 and 6.8, the polypeptides according to the invention specifically bind to FcRn with an off rate constant (k_{off}) selected from the group consisting of at most about 10⁻¹s⁻¹, at most about 10⁻² s⁻¹, at most about 10⁻³s⁻¹, of at most about 10⁴s⁻¹, at most about 10⁻⁵ s⁻¹, and at most about 10⁻⁶ s⁻¹, preferably as measured by surface plasmon resonance or BLI.

In particular embodiments, at an acidic pH of between 5.0 and 6.8, the polypeptides according to the invention specifically bind to FcRn with an off rate constant (k_{off}) selected from the group consisting of at most about 10⁻¹s⁻¹, at most about 10⁻²s⁻¹, at most about 10⁻³s⁻¹, of at most about 10⁻⁴s⁻¹, at most about 10⁻⁵s⁻¹, and at most about 10⁻⁶s⁻¹. The off rate constant (k_{off}) of these amino acid sequences and polypeptides for FcRn at acidic pH, preferably at a pH of between 5.0 and 6.8 is at least three times lower than the off rate constant (k_{off}) of the same amino acid sequences and polypeptides for FcRn at neutral or physiologic pH of 7.4. In further particular embodiments, at an acidic pH of between 5.0 and 6.8, the polypeptides according to the invention bind to FcRn with off rate constant (k_{off}) that is at least ten times lower than the off rate constant (k_{off}) for FcRn of the same polypeptides at neutral or physiologic pH of 7.4. In yet further particular embodiments, at an acidic pH of between 5.0 and 6.8, the polypeptides according to the invention bind to FcRn with off rate constant (k_{off}) that is at least fifty times lower, such as at least hundred times lower than the off rate constant (k_{off}) for FcRn of the same polypeptides at neutral or physiologic pH of 7.4.

In certain particular embodiments, the present invention provides polypeptides as described herein characterized in that the at least one ISVD binds to FcRn at neutral or physiologic pH of 7.4 with an off rate constant (k_{off}) that is at least three times, such as at least ten times, such as at least fifty times, such as at least hundred times higher than the off rate constant (k_{off}) with which the at least one ISVD binds to FcRn at acidic pH of between 5.0 and 6.8.

In certain particular embodiments, the present invention provides polypeptides as described herein characterized in that the at least one ISVD does not detectably, selectively or specifically bind to FcRn, or exhibits no or essentially no binding to FcRn at neutral or physiologic pH, such as at a pH of 7.4.

Accordingly, the present invention provides improved FcRn binding agents that can be used for various applications, including but not limited to prolonging the in vivo half-life of (existing or future) therapeutic compounds. In particular embodiments, the polypeptides of the invention have a high affinity for a unique conformational epitope on FcRn, that is different from the epitopes on FcRn bound by serum albumin and IgG.

In particular embodiments, the polypeptides of the invention have a high affinity for a particular epitope on FcRn at an acidic pH of between 5.0 and 6.8, while showing a reduced binding or not showing any detectable, selective and/or specific binding to FcRn at physiological or neutral pH of 7.4

In certain particular embodiments, the polypeptides of the present invention have a molecular weight of at least 30 kDa, and in particular have a molecular weight of between 30 kDa and 100 kDa. According to these particular embodiments, the compounds of the present invention can be most effectively and optimally applied for several applications, in particular for in vivo half-life extension of therapeutic molecules.

Thus, in particular embodiments, the present invention provides polypeptides comprising: at least one ISVD specifically binding to FcRn in a pH dependent manner, and at least one further moiety, characterized in that
a) the at least one ISVD specifically binds to FcRn in a pH-dependent manner, such that the binding affinity at a pH between 5.0 and 6.8 is at least three times higher than the binding affinity at a pH of 7.4, and
b) the polypeptide has a molecular weight of at least 30 kDa.

In certain further particular embodiments, the polypeptide has a molecular weight of between about 30 kDa and 200 kDa, such as between about 30 kDa and 100 kDa.

### 5.3 Multi-Specific Polypeptides

Generally, polypeptides according to the invention that comprise or essentially consist of a single building block, single immunoglobulin single variable domain or single Nanobody® ISVD will be referred to herein as “monovalent” proteins or polypeptides, as “monovalent constructs”, as “monovalent building block”, as “monovalent immunoglobulin single variable domain”, “monovalent Nanobody® ISVD” or as “monovalent Nanobody® V_{HH}”, respectively.

Polypeptides that comprise or essentially consist of two or more immunoglobulin single variable domains (such as at least two immunoglobulin single variable domains of the invention) will be referred to herein as “multivalent” polypeptides, (fusion) proteins, compounds or as “multivalent constructs”. Some non-limiting examples of such multivalent constructs will become clear from the further description herein.

Polypeptides of the invention that contain at least two building blocks, ISVDs, Nanobody® ISVDs or Nanobody® V_{HH}s, in which at least one building block, ISVD, Nanobody® ISVD or Nanobody® V_{HH} is directed against a first antigen (i.e., against the first target, such as, e.g., FcRn) and at least one building block, ISVD, Nanobody® ISVD or Nanobody® V_{HH} is directed against a second antigen (i.e., against the second target which is different from the first target, such as, e.g., a serum albumin or a therapeutic target other than FcRn), will also be referred to as “multispecific” polypeptides of the invention, and the building blocks, ISVDs, Nanobody® ISVDs or Nanobody® V_{HH}s present in such polypeptides will also be referred to herein as being in a “multivalent format” or “multispecific format”. Thus, for example, a “bispecific” polypeptide of the invention is a polypeptide that comprises at least one building block, ISVD, Nanobody® ISVD or Nanobody® V_{HH} directed against a first target (e.g. FcRn) and at least one further building block, ISVD, Nanobody® ISVD or Nanobody® V_{HH} directed against a second target (i.e., directed against a second target different from said first target, such as, e.g., serum albumin), whereas a “trispecific” polypeptide of the invention is a polypeptide that comprises at least one building block, ISVD, Nanobody® ISVD or Nanobody® V_{HH} directed against a first target (e.g., FcRn), a second building block, ISVD, Nanobody® ISVD or Nanobody® V_{HH} directed against a second target different from said first target (e.g., serum albumin) and at least one further building block, ISVD, Nanobody ISVD or Nanobody® V_{HH} directed against a third antigen (i.e., different from both the first and the second target, such as a therapeutic target other than FcRn and serum albumin) etc. As will be clear from the description, the invention is not limited to bispecific polypeptides, in the sense that a multispecific polypeptide of the invention may comprise at least a first building block, ISVD, Nanobody® ISVD or Nanobody® V_{HH} against a first target, a second building block, ISVD, Nanobody® ISVD or Nanobody® V_{HH} against a second target and any number of building blocks, ISVDs, Nanobody® ISVDs or Nanobody® V_{HH}s directed against one or more targets, which may be the same or different from the first and/or second target, respectively.

The terms bispecific polypeptide, bispecific format, bispecific construct, bispecific Nanobody® construct, bispecific and bispecific ISVD construct are used interchangeably herein.

As will be clear from the further description above and herein, the immunoglobulin single variable domains of the invention can be used as “building blocks” to form polypeptides of the invention, e.g., by suitably combining them with other groups, residues, moieties or binding units, in order to form compounds or constructs as described herein (such as, without limitations, the bi-/tri-/tetra-/multivalent and bi-/tri-/tetra-/multispecific polypeptides of the invention described herein) which combine within one molecule one or more desired properties or biological functions.

In further specific embodiments, the present invention provides polypeptides wherein said at least one ISVD and said at least one further binding moiety are directly linked to each other or are linked via linkers.

The compounds, constructs or polypeptides of the invention can generally be prepared by a method which comprises at least one step of suitably linking the one or more immunoglobulin single variable domains of the invention to the one or more further groups, residues, moieties or binding units, optionally via one or more suitable linkers, so as to provide the compound, construct or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein. The process of designing/selecting and/or preparing a compound, construct or polypeptide of the invention, starting from a polypeptide comprising at least one ISVD of the invention, is also referred to herein as “formatting” said polypeptide of the invention; and a polypeptide of the invention that is made part of a compound, construct or polypeptide of the invention is said to be “formatted” or to be “in the format of” said compound, construct or polypeptide of the invention. Examples of ways in which a polypeptide of the invention can be formatted and examples of such formats will be clear to the skilled person based on the disclosure herein; and such formatted immunoglobulin single variable domains or polypeptides form a further aspect of the invention.

For example, such further groups, residues, moieties or binding units may be one or more additional immunoglobulins, such that the compound or construct is a (fusion) protein or (fusion) polypeptide. In a preferred but non-limiting aspect, said one or more other groups, residues, moieties or binding units are immunoglobulin single variable domains (ISVDs). Even more preferably, said one or more other groups, residues, moieties or binding units are chosen from the group consisting of domain antibodies, immunoglobulin single variable domains that are suitable for use as a domain antibody, single domain antibodies, immunoglobulin single variable domains (ISVDs) that are suitable for use as a single domain antibody, “dAb” 's, immunoglobulin single variable domains that are suitable for use as a dAb, V_{HH}s, humanized V_{HH}s, camelized V_{H}S, or Nanobody® V_{HH}s. Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domains or polypeptides of the invention so as to provide a “derivative” of an ISVD or polypeptide of the invention, as further described herein.

Also within the scope of the present invention are compounds or constructs, which comprise or essentially consist of one or more derivatives as described herein, and optionally further comprise one or more other groups, residues, moieties or binding units, optionally linked via one or more linkers. Preferably, said one or more other groups, residues, moieties or binding units are immunoglobulin single variable domains. In the compounds or constructs described above, the one or more immunoglobulin single variable domains of the invention and the one or more groups, residues, moieties or binding units may be linked directly to each other and/or via one or more suitable linkers or spacers. For example, when the one or more groups, residues, moieties or binding units are immunoglobulin single variable domains, the linkers may also be immunoglobulin single variable domains, so that the resulting compound or construct is a fusion protein or fusion polypeptide.

In some embodiments, the polypeptides comprise at least two or more immunoglobulin single variable domains disclosed herein. In some embodiments, the polypeptides essentially consist of two or more immunoglobulin single variable domains disclosed herein. A polypeptide that “essentially consists of” two or more immunoglobulin single variable domains, is a polypeptide that in addition to the two or more immunoglobulin single variable domains disclosed herein does not have additional immunoglobulin single variable domains. For instance, a polypeptide that essentially consists of two immunoglobulin single variable domains does not include any additional immunoglobulin single variable domains. However, it should be appreciated that a polypeptide that essentially consists of two or more immunoglobulin single variable domains may include additional functionalities, such as a label, a toxin, one or more linkers, a binding sequence, etc. These additional functionalities include both amino acid based and non-amino acid based groups. In some embodiments, the polypeptides consist of one or more immunoglobulin single variable domains disclosed herein. It should be appreciated that the terms “polypeptide construct” and “polypeptide” can be used interchangeably herein (unless the context clearly dictates otherwise).

In some embodiments, the polypeptides include multivalent or multispecific constructs comprising immunoglobulin single variable domains disclosed herein. In some embodiments, the polypeptides comprise one or more antibody-based scaffolds and/or non-antibody-based scaffolds disclosed herein. In some embodiments, the polypeptides comprise a serum binding protein moiety. In some embodiments, the serum binding protein moiety is an immunoglobulin single variable domain. In some embodiments, the immunoglobulin single variable domain is a Nanobody® V_{HH}.

It will be appreciated that the order of the building blocks, such as e.g. a first building block, a second building block, a third building block etc., on the polypeptide (orientation) can be chosen according to the needs of the person skilled in the art, as well as the relative affinities which may depend on the location of these building blocks in the polypeptide. Whether the polypeptide comprises a linker, is a matter of design choice. However, some orientations, with or without linkers, may provide preferred binding characteristics in comparison to other orientations. For instance, the order of a first and a second building block in the polypeptide of the invention can be (from N-terminus to C-terminus): (i) first building block (e.g., a first ISVD such as a first Nanobody® V_{HH})—[linker]—second building block (e.g. a second ISVD such as a second Nanobody® V_{HH}); or (ii) second building block (e.g., a second ISVD such as a second Nanobody® V_{HH})—[linker]—first building block (e.g., a first ISVD such as a first Nanobody® V_{HH}); (wherein the linker is optional). All orientations are encompassed by the invention.

The polypeptides according to the present invention comprising at least one ISVD and at least one other binding moiety, are such that the at least one ISVD and the at least one other binding moiety are directly linked to each other or are linked via a linker, such as peptidic linkers. The use of linkers to connect two or more (poly)peptides is well known in the art. One frequently used class of peptidic linkers are known as the “Gly-Ser” or “GS” linkers. These are linkers that essentially consist of glycine (G) and serine (S) residues, and usually comprise one or more repeats of a peptide motif such as the GGGGS (SEQ ID NO: 86) motif (for example, exhibiting the formula (Gly-Gly-Gly-Gly-Ser), in which n may be 1, 2, 3, 4, 5, 6, 7 or more). Some often used examples of such GS linkers are 9GS linkers (GGGGSGGGS, SEQ ID NO: 87), 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In particular but non-limiting embodiments, the linker is chosen from the group consisting of linkers of 3A, 3GS, 5GS, 7GS, 9GS, 10GS, 15GS, 18GS, 20GS, 25GS, 30GS and 35GS (SEQ ID NOs: 37 to 48). In other particular embodiment, the linker may be the IgG hinge region, as the IgG1 hinge region (SEQ ID NO.: 50 or 126). Preferably, the linker may be the IgG1 short hinge region (SEQ ID NO.: 126) and/or the 35GS linker (SEQ ID NO.: 48).

TABLE A-2
Linker sequences (″ID″ refers to the SEQ ID NO as used herein)

| Name | ID | Amino acid sequence |
|---|---|---|
| 3A linker | 37 | AAA |
| 5GS linker | 38 | GGGGS |
| 7GS linker | 39 | SGGSGGS |
| 8GS linker | 40 | GGGGSGGS |
| 9GS linker | 41 | GGGGSGGGS |
| 10GS linker | 42 | GGGGSGGGGS |
| 15GS linker | 43 | GGGGSGGGGSGGGGS |
| 18GS linker | 44 | GGGGSGGGGSGGGGSGGS |
| 20GS linker | 45 | GGGGSGGGGSGGGGSGGGGS |
| 25GS linker | 46 | GGGGSGGGGSGGGGSGGGGSGGGGS |
| 30GS linker | 47 | GGGGSGGGGSGGGGSGGGGSGGGGSGGGGS |
| 35GS linker | 48 | GGGGSGGGGSGGGGSGGGGSGGGGSGGGGSGGGGS |
| 40GS linker | 49 | GGGGSGGGGSGGGGSGGGGSGGGGSGGGGSGGGGSGGGGS |
| G1 hinge | 50 | EPKSCDKTHTCPPCP |
| 9GS-G1 hinge | 51 | GGGGSGGGSEPKSCDKTHTCPPCP |
| Llama upper long hinge | 52 | EPKTPKPQPAAA |
| region | | |
| G3 hinge | 53 | ELKTPLGDTTHTCPRCPEPKSCDTPPPCPRCPEPKSCDTPPPCPRCP |
| | | EPKSCDTPPPCPRCP |
| Short G1 hinge | 126 | DKTHTCPPCP |

In certain further particular embodiments, the at least one further moiety is a protein moiety, such as a further ISVD.

In certain further particular embodiments, the at least one further moiety is a protein moiety, such as a serum protein, in particular but not limited to serum albumin.

In certain further particular embodiments, the at least one further moiety is a protein binding moiety, such as a serum protein binding moiety, such as a serum albumin binding moiety, in particular a serum protein binding ISVD, more particularly a serum albumin binding ISVD.

In certain further particular embodiments, the at least one serum albumin binding moiety specifically binds to amino acid residues on serum albumin that are not involved in binding of serum albumin to FcRn.

In certain further particular embodiments, the present invention provides polypeptides as described herein, characterized in that the at least one ISVD specifically binding to serum albumin is a (single) domain antibody, a V_{HH}, a Nanobody® V_{HH}, a humanized V_{HH}, or a camelized V_{H}.

In certain further particular embodiments, the at least one further moiety is at least one ISVD binding to serum albumin.

In certain further particular embodiments, the at least one further moiety is at least one ISVD binding to serum albumin, which essentially consists of 4 framework regions (FR1 to FR4, respectively) and 3 complementarity determining regions (CDR1 to CDR3 respectively), and in which CDR1 is SFGMS (SEQ ID NO: 16), CDR2 is SISGSGSDTLYADSVKG (SEQ ID NO: 17) and CDR3 is GGSLSR (SEQ ID NO: 18), CDR determined according to Kabat definition; and/or in which CDR1 is GFTFRSFGMS (SEQ ID NO: 19), CDR2 is SISGSGSDTL (SEQ ID NO: 20) and CDR3 is GGSLSR (SEQ ID NO: 21), CDR determined according to AbM definition (Kontermann et al., 2010).

The international application WO 06/122787 describes a number of ISVDs binding to (human) serum albumin. These ISVDs include the Nanobody® V_{HH} called Alb-1 (SEQ ID NO: 52 in WO 06/122787) and humanized variants thereof, such as Alb-8 (SEQ ID NO: 62 in WO 06/122787). Moreover, WO 2012/175400 describes a further improved version of Alb-1, called Alb-23.

In particular embodiments, the polypeptides of the present invention comprise at least one ISVD binding to the epitope on FcRn as disclosed herein and further comprise a serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown on pages 7-9 of WO 2012/175400 and the albumin binders described in WO 2012/175741, WO 2015/173325, WO 2017/080850, WO 2017/085172, WO 2018/104444, WO 2018/134235, WO 2018/134234. Some preferred serum albumin binders are also shown in Table A-3.

TABLE A-3
Serum albumin binding ISVD sequences (″ID″ refers to the SEQ ID NO as used herein)

| Name | ID | Amino acid sequence |
|---|---|---|
| Alb8 | 22 | EVQLVESGGGLVQPGNSLRLSCAASGFTFSSFGMSWVRQAPGKGLEWVSSISGSGSDT |
| | | LYADSVKGRFTISRDNAKTTLYLQMNSLRPEDTAVYYCTIGGSLSRSSQGTLVTVSS |
| Alb23 | 23 | EVQLLESGGGLVQPGGSLRLSCAASGFTFRSFGMSWVRQAPGKGPEWVSSISGSGSDT |
| | | LYADSVKGRFTISRDNSKNTLYLQMNSLRPEDTAVYYCTIGGSLSRSSQGTLVTVSS |
| Alb129 | 24 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTATYYCTIGGSLSRSSQGTLVTVSSA |
| Alb132 | 25 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTATYYCTIGGSLSRSSQGTLVTVSSA |
| Alb11 | 26 | EVQLVESGGGLVQPGNSLRLSCAASGFTFSSFGMSWVRQAPGKGLEWVSSISGSGSDT |
| | | LYADSVKGRFTISRDNAKTTLYLQMNSLRPEDTAVYYCTIGGSLSRSSQGTLVTVSS |
| Alb11 | 27 | EVQLVESGGGLVQPGNSLRLSCAASGFTFSSFGMSWVRQAPGKGLEWVSSISGSGSDT |
| (S112K)-A | | LYADSVKGRFTISRDNAKTTLYLQMNSLRPEDTAVYYCTIGGSLSRSSQGTLVKVSSA |
| Alb82 | 28 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTALYYCTIGGSLSRSSQGTLVTVSS |
| Alb82-A | 29 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTALYYCTIGGSLSRSSQGTLVTVSSA |
| Alb82-AA | 30 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTALYYCTIGGSLSRSSQGTLVTVSSAA |
| Alb82- | 31 | EVQLVESGGGVVQPGNSLRLSCAASGFTFSSFGMSWVRQAPGKGLEWVSSISGSGSDT |
| AAA | | LYADSVKGRFTISRDNAKTTLYLQMNSLRPEDTALYYCTIGGSLSRSSQGTLVTVSSAAA |
| Alb82-G | 32 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTALYYCTIGGSLSRSSQGTLVTVSSG |
| Alb82-GG | 33 | EVQLVESGGGVVQPGNSLRLSCAASGFTFSSFGMSWVRQAPGKGLEWVSSISGSGSDT |
| | | LYADSVKGRFTISRDNAKTTLYLQMNSLRPEDTALYYCTIGGSLSRSSQGTLVTVSSGG |
| Alb82- | 34 | EVQLVESGGGVVQPGNSLRLSCAASGFTFSSFGMSWVRQAPGKGLEWVSSISGSGSDT |
| GGG | | LYADSVKGRFTISRDNAKTTLYLQMNSLRPEDTALYYCTIGGSLSRSSQGTLVTVSSGGG |
| Alb23002 | 35 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTALYYCTIGGSLSRSSQGTLVTVSS |
| Alb223 | 36 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTALYYCTIGGSLSRSSQGTLVTVSSA |

In particular embodiments of the present invention, the polypeptides of the present invention comprise at least one ISVD binding to the epitope on FcRn as disclosed herein and further comprise a serum albumin binding ISVD having the full amino acid sequence of ALB23002 (SEQ ID NO: 35, see Table A-3).

Also in a preferred embodiment, the amino acid sequence of an ISVD binding to human serum albumin may have a sequence identity of more than 90%, such as more than 95% or more than 99%, with SEQ ID NO: 35, wherein optionally the CDRs are as defined above as SEQ ID NO's: 16 to 18 (according to Kabat) or SEQ ID NO's: 19 to 21 (according to AbM). In particular, the ISVD binding to human serum albumin preferably has the amino acid sequence of SEQ ID NO: 35.

When such an ISVD binding to human serum albumin has 2 or 1 amino acid difference in at least one CDR relative to a corresponding reference CDR sequence (as defined above as SEQ ID NO: 16 to 18 (according to Kabat) or SEQ ID NO's: 19 to 21 (according to AbM)), the ISVD preferably has at least half the binding affinity, and preferably at least the same binding affinity, to human serum albumin as construct ALB23002, wherein the binding affinity is measured using the same method, such as SPR.

When such an ISVD binding to human serum albumin has a C-terminal position it may exhibit a C-terminal alanine (A) or glycine (G) extension and is preferably selected from SEQ ID NOs: 24, 25, 27, 29, 30, 31, 32, 33, 34 and 36 (see table A-3). In a preferred embodiment, the ISVD binding to human serum albumin has another position than the C-terminal position (i.e., is not the C-terminal ISVD of the polypeptide of the invention) and is selected from SEQ ID NOs: 22, 23, 26, 28, and 35 (see table A-3).

In particular embodiments, the polypeptides of the invention comprise at least a first and at least a second immunoglobulin single variable domain (ISVD), wherein said at least first ISVD has high affinity for/binds specifically to FcRn at acidic pH and said at least second ISVD has high affinity for/binds specifically to serum albumin. Preferred sequences of polypeptides, polypeptide constructs, ISVD building blocks and combinations of CDR sequences and FR sequences according to particular embodiments of the present invention are shown in Tables A-4 to A-8.

TABLE A-4
Configuration of multivalent ISVD constructs according
to particular embodiments of the present invention

| | SEQ | Building | | Building |
|---|---|---|---|---|
| Name | ID NO. | block 1 | Linker | block 2 |
| T0263018B11 | 122 | T0263018B11 | 35GS | ALB23002 |
| (parent)-9GS-ALB23002 | | | | |
| T0263018B11 | 123 | T0263018B11 | 35GS | ALB23002 |
| (W100cS)-9GS-ALB23002 | | (W100cS) | | |
| ALB23002-9GS-T0263018B11 | 124 | ALB23002 | 35GS | T0263018B11 |
| (parent) | | | | |
| ALB23002-9GS-T0263018B11 | 125 | ALB23002 | 35GS | T0263018B11 |
| (W100cS) | | | | (W100cS) |

TABLE A-5
Amino acid sequences of the different monovalent VHH building blocks used within the multi-
specific polypeptides according to the present invention (″ID″ refers to the SEQ ID NO. as
used herein)

| Name | ID | Amino acid sequence |
|---|---|---|
| T0263018B11 | 14 | EVQLVESGGGVVQPGGSLRLSCAASGFTFSSYAMYWVRQAPGKGLEWVSAISSGGGSTDYADSVKGRFTI |
| (parent) | | SRDNSKNTLYLQMNSLRPEDTALYYCAADTLYTSLTWYSYWGQGTLVTVSS |
| T0263018B11 | 15 | EVQLVESGGGVVQPGGSLRLSCAASGFTFSSYAMYWVRQAPGKGLEWVSAISSGGGSTDYADSVKGRFTI |
| (W100cS) | | SRDNSKNTLYLQMNSLRPEDTALYYCAADTLYTSLTSYSYWGQGTLVTVSS |
| ALB23002 | 35 | EVQLVESGGGVVQPGGSLRLSCAASGFTFRSFGMSWVRQAPGKGPEWVSSISGSGSDTLYADSVKGRFTI |
| | | SRDNSKNTLYLQMNSLRPEDTALYYCTIGGSLSRSSQGTLVTVSS |

TABLE A-6
Sequences for CDRs according to AbM numbering and frameworks (″ID″ refers to the given SEQ ID NO.) CDR3 ID FR4 ID

| Building | | | | | | | | | | | | | | | |
|---|---|---|---|---|---|---|---|---|---|---|---|---|---|---|---|
| block | ID | FR1 | ID | CDR1 | ID | FR2 | ID | CDR2 | ID | FR3 | ID | CDR3 | ID | FR4 | ID |
| T0263018B11 | 14 | EVQLVESGGG | 114 | GFTFSSYAMY | 11 | WVRQAPGKGLE | 115 | AISSG | 12 | YADSVKGRFTISRDN | 116 | DTLYTSLTWYSY | 105 | WGQGTLVTVSS | 117 |
| (parent) | | VVQPGGSLRLS | | | | WVS | | GGSTD | | SKNTLYLQMNSLRP | | | | | |
| | | CAAS | | | | | | | | EDTALYYCAA | | | | | |
| T0263018B11 | 15 | EVQLVESGGG | 114 | GFTFSSYAMY | 11 | WVRQAPGKGLE | 115 | AISSG | 12 | YADSVKGRFTISRDN | 116 | DTLYTSLTSYSY | 13 | WGQGTLVTVSS | 117 |
| (W100cS) | | VVQPGGSLRLS | | | | WVS | | GGSTD | | SKNTLYLQMNSLRP | | | | | |
| | | CAAS | | | | | | | | EDTALYYCAA | | | | | |
| ALB23002 | 35 | EVQLVESGGG | 118 | GFTFRSFGMS | 19 | FGMSWVRQAPG | 119 | SISGSG | 20 | YADSVKGRFTISRDN | 120 | GGSLSR | 21 | SSQGTLVTVSS | 121 |
| | | VVQPGGSLRLS | | | | KGPEWVS | | SDTL | | SKNTLYLQMNSLRP | | | | | |
| | | CAAS | | | | | | | | EDTALYYCTI | | | | | |

TABLE A-7
Sequences for CDRs according to Kabat numbering and frameworks (″ID″ refers to the given SEQ ID NO.)

| Building | | | | | | | | | | | | | | | |
|---|---|---|---|---|---|---|---|---|---|---|---|---|---|---|---|
| block | ID | FR1 | ID | CDR1 | ID | FR2 | ID | CDR2 | ID | FR3 | ID | CDR3 | ID | FR4 | ID |
| T0263018B11 | 14 | EVQLVESGGG | 106 | SYAMY | 8 | WVRQAPG | 107 | AISSG | 9 | RFTISRDNSK | 108 | DTLYTSLT | 104 | WGQGTLV | 109 |
| (parent) | | VVQPGGSLRL | | | | KGLEWVS | | GGSTD | | NTLYLQMNS | | WYSY | | TVSS | |
| | | SCAASGFTFS | | | | | | YADSV | | LRPEDTALYY | | | | | |
| | | | | | | | | KG | | CAA | | | | | |
| T0263018B11 | 15 | EVQLVESGGG | 106 | SYAMY | 8 | WVRQAPG | 107 | AISSG | 9 | RFTISRDNSK | 108 | DTLYTSLTS | 10 | WGQGTLV | 109 |
| (W100cS) | | VVQPGGSLRL | | | | KGLEWVS | | GGSTD | | NTLYLQMNS | | YSY | | TVSS | |
| | | SCAASGFTFS | | | | | | YADSV | | LRPEDTALYY | | | | | |
| | | | | | | | | KG | | CAA | | | | | |
| ALB23002 | 35 | EVQLVESGGG | 110 | SFGMS | 16 | WVRQAPG | 111 | SISGSG | 17 | RFTISRDNSK | 112 | GGSLSR | 18 | SSQGTLVT | 113 |
| | | VVQPGGSLRL | | | | KGPEWVS | | SDTLY | | NTLYLQMNS | | | | VSS | |
| | | SCAASGFTFR | | | | | | ADSVK | | LRPEDTALYY | | | | | |
| | | | | | | | | G | | CTI | | | | | |

TABLE A-8
Amino acid sequences of selected multivalent polypeptide (″ID″ refers to the given SEQ ID NO.)

| Name | ID | Amino acid sequence |
|---|---|---|
| T0263018B11 (parent)- | 122 | EVQLVESGGGVVQPGGSLRLSCAASGFTFSSYAMYWVRQAPGKGLEWVSAISSGGGS |
| 9GS-ALB23002 | | TDYADSVKGRFTISRDNSKNTLYLQMNSLRPEDTALYYCAADTLYTSLTWYSYWGQ |
| | | GTLVTVSSGGGGSGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLRPEDTAL |
| | | YYCTIGGSLSRSSQGTLVTVSS |
| T0263018B11 (W100cS)- | 123 | EVQLVESGGGVVQPGGSLRLSCAASGFTFSSYAMYWVRQAPGKGLEWVSAISSGGGS |
| 9GS-ALB23002 | | TDYADSVKGRFTISRDNSKNTLYLQMNSLRPEDTALYYCAADTLYTSLTSYSYWGQ |
| | | GTLVTVSSGGGGSGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLRPEDT |
| | | ALYYCTIGGSLSRSSQGTLVTVSS |
| ALB23002-9GS- | 124 | EVQLVESGGGVVQPGGSLRLSCAASGFTFRSFGMSWVRQAPGKGPEWVSSISGSGSDTL |
| T0263018B11 | | YADSVKGRFTISRDNSKNTLYLQMNSLRPEDTALYYCTIGGSLSRSSQGTLVTVSS |
| (parent) | | GGGGSGGGSEVQLVESGGGVVQPGGSLRLSCAASGFTFSSYAMYWVRQAPGKG |
| | | LEWVSAISSGGGSTDYADSVKGRFTISRDNSKNTLYLQMNSLRPEDTALYYCAAD |
| | | TLYTSLTWYSYWGQGTLVTVSS |
| ALB23002-9GS- | 125 | EVQLVESGGGVVQPGGSLRLSCAASGFTFRSFGMSWVRQAPGKGPEWVSSISGSGSDTL |
| T0263018B11 | | YADSVKGRFTISRDNSKNTLYLQMNSLRPEDTALYYCTIGGSLSRSSQGTLVTVSS |
| (W100cS) | | GGGGSGGGSEVQLVESGGGVVQPGGSLRLSCAASGFTFSSYAMYWVRQAPGK |
| | | GLEWVSAISSGGGSTDYADSVKGRFTISRDNSKNTLYLQMNSLRPEDTALYYCAA |
| | | DTLYTSLTSYSYWGQGTLVTVSS |

In particular embodiments, the present invention provides polypeptides as described herein, characterized in that the polypeptides further comprise a therapeutic moiety, which comprises a (single) domain antibody, a Nanobody® V_{HH}, a V_{HH}, a humanized V_{HH} or a camelized V_{H}.

In particular embodiments, the polypeptides of the invention comprise at least a first, at least a second and at least a third immunoglobulin single variable domain (ISVD), wherein said at least a first ISVD has high affinity for/binds specifically to FcRn at acidic pH; said at least a second ISVD has high affinity for/binds specifically to serum albumin, and said at least a third ISVD has high affinity for/binds specifically to a therapeutically relevant antigen other than FcRn and serum albumin.

It will be appreciated (as is also demonstrated in the Example section) that the ISVD binding to FcRn and the ISVD binding to serum albumin and/or the ISVD binding to a therapeutic target other than FcRn and albumin, can be positioned in any order in the polypeptides of the invention. More particularly, in one embodiment, the ISVD binding FcRn is positioned N-terminally and the ISVD binding another antigen is positioned C-terminally. In another embodiment, the ISVD binding the other antigen is positioned N-terminally and the ISVD binding FcRn is positioned C-terminally.

The invention further provides compounds or constructs, and in particular proteins or polypeptides that comprise or essentially consist of one or more ISVDs or polypeptides of the invention, and optionally further comprise one or more other groups, residues, moieties or binding units. As will become clear to the skilled person from the further disclosure herein, such further groups, residues, moieties, binding units or amino acid sequences may or may not provide further functionality to the polypeptide of the invention (and/or to the compound or construct in which it is present) and may or may not modify the properties of the polypeptide of the invention.

In particular embodiments, the at least one further moiety is an Fc region of an immunoglobulin (Ig). Thus, in certain particular embodiments, the invention provides at least one ISVD specifically binding to FcRn in a pH dependent manner, and at least one further moiety, characterized in that:
a) the at least one ISVD specifically binds to FcRn in a pH-dependent manner, such that the binding affinity at a pH between 5.0 and 6.8 is at least three times higher than the binding affinity at a pH of 7.4,
b) the polypeptide has a molecular weight of at least 30 kDa, in particular between about 30 kDa and 100 kDa, and
c) the at least one further moiety is an Fc region or an Fc domain of an immunoglobulin (Ig).

In particular embodiments, the present invention provides polypeptides as described herein, wherein the at least one further moiety is an Fc region or an Fc domain of an immunoglobulin G (IgG). In further particular embodiments, the present invention provides polypeptides as described herein, wherein the at least one further moiety is an Fc region or an Fc domain of an immunoglobulin G type 4 (IgG4).

In particular embodiments, the present invention provides polypeptides as described herein, wherein the at least one further moiety is an Fc region or an Fc domain of an immunoglobulin A (IgA).

In some particular embodiments, the Fc domain is a wild-type Fc domain of an immunoglobulin. The wild-type Fc domain may bind FcRn with K_{D} values of more than 500 nM, e.g., more than 600 nM.

In certain other particular embodiments, the Fc domain can be a mutant Fc domain of an immunoglobulin that binds FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In certain other particular embodiments, the Fc domain can be a mutant Fc domain that does not detectably, selectively or specifically bind to FcRn, or exhibits no or essentially no binding to FcRn neither at a pH of between 5.0 and 6.8 nor at neutral or physiologic pH, such as at a pH of 7.4.

Suitable constructs and formats comprising at least one polypeptide according to the present invention and at least one Fc domain will become clear from the further description herein. Mutant variants of suitable Fc domains as well as suitable formats of Fc domain constructs are well known in the art and are inter alia described in published patent applications EP 2654790, U.S. Ser. No. 10/239,944, US 20120251531, U.S. Pat. No. 9,133,274, WO 2014065945, WO 2015150447 and WO 2021016571.

Hence, as described above, the polypeptides according to the present invention further comprise an Fc domain of an IgG. Fc domain of an IgG refers to the C-terminal non-antigen binding region of an immunoglobulin G heavy chain that contains at least a portion of the constant region. In particular embodiments, the Fc domain can be a native Fc region, i.e., as it occurs in natural antibodies, or it can be a variant Fc region, comprising one or more alterations, mutations or variations as compared to the native Fc domain, In particular embodiments, an Fc domain of an IgG can also be a fragment of a native Fc domain or a fragment of a variant Fc domain. In certain embodiments, a polypeptide as described herein comprises a native Fc domain of a human IgG, such as preferably a native Fc of human IgG4 (e.g. Uniprot sequence P01861, SEQ ID NO.: 129). In other embodiments, the polypeptides according to the present invention comprise variant Fc domains which have altered binding properties for an Fc ligand relative to an unmodified parent Fc molecule. For example, a polypeptide described herein may comprise an Fc region having one or more of amino acid residues 234, 235, 236, 237, 297, 318, 320 and 322 substituted to a different amino acid residue, such that the variant Fc region has an altered affinity for an effector ligand, e.g., an Fc receptor or the C1 component of complement, as described in U.S. Pat. Nos. 5,624,821 and 5,648,260, both to Winter et al.

In particular embodiments, the polypeptides of the present invention comprise an Fc variant domain with reduced effector function, in particular the so-called “FALA” or “LALA” Fc mutant with substitution of residues 234 and 235 to alanine. Extra optional mutations include the substitution of arginine residue 409 to lysine, deletion of lysine residue 447. In other embodiments, the Fc variant domain comprises the so-called “FALA” mutations as described herein and the mutation S228P. These Fc variants are referred to as “pFALA”.

In particular embodiments, the polypeptides according to the present invention comprise an Fc variant domain showing improved binding to the FcRn receptor compared to the native Fc domain. Such Fc variants include those with substitutions at one or more of Fc region residues 259, 308, 428, and 434. Other variants that increase Fc binding to FcRn include: 250E, 250Q, 428L, 428F, 250Q/428L (Hinton et al., 2004, J. Biol. Chem. 279(8): 6213-6216, Hinton et al., 2006, Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276(9):6591-6604).

In certain particular embodiments, the polypeptides according to the present invention comprise an Fc variant domain wherein methionine 428 was substituted to lysine and asparagine 434 was substituted to serine.

In particular embodiments, the polypeptides according to the present invention comprise an Fc variant domain showing reduced or no binding to the FcRn receptor compared to the native Fc domain. Such Fc variants include those with substitutions at one or more of Fc region residues 253, 310 and 453.

In particular embodiments, the polypeptides according to the present invention comprise an Fc variant domain wherein isoleucine 428 was substituted to alanine, histidine 310 was substituted to alanine, and histidine 453 was substituted to alanine, optionally in combination with histidine 453 substituted to alanine.

In particular embodiments, the at least one further moiety is a non-protein moiety, such as but not limited to a poly-ethylene-glycol (PEG) moiety.

Thus, in certain particular embodiments, the invention provides at least one ISVD specifically binding to FcRn in a pH dependent manner, and at least one further moiety, characterized in that:
a) the at least one ISVD specifically binds to FcRn in a pH-dependent manner, such that the binding affinity at a pH between 5.0 and 6.5 is at least three times higher than the binding affinity at a pH of 7.4, and
b) the polypeptide has a molecular weight of at least 30 kDa, in particular between about 30 kDa and 100 kDa,
c) the at least one further moiety is a non-protein moiety, such as but not limited to a poly-ethylene-glycol (PEG) moiety.

In particular embodiments, the present invention provides polypeptides as described herein, characterized in that the polypeptides further comprise a therapeutic moiety.

In particular embodiments, the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of a binding domain or binding unit, such as an immunoglobulin or immunoglobulin sequence (including but not limited to a fragment of an immunoglobulin), such as an antibody or an antibody fragment (including but not limited to an ScFv fragment), or of another suitable protein scaffold, such as protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23: 1257), and binding moieties based on DNA or RNA including but not limited to DNA or RNA aptamers (Ulrich et al., Comb Chem High Throughput Screen 2006 9(8):619-32).

In yet another aspect, the at least one therapeutic moiety comprises or essentially consists of an antibody variable domain, such as a heavy chain variable domain or a light chain variable domain.

In a preferred aspect, the at least one therapeutic moiety comprises or essentially consists of at least one immunoglobulin single variable domain, such as a domain antibody, single domain antibody, “dAb” or a V_{HH} (such as a Nanobody® V_{HH}, a humanized V_{HH} or a camelized V_{H}) or an IgNAR domain.

For example and without limitation, such polypeptides, compounds or constructs of the invention may comprise:
at least one FcRn binder of the invention and at least one ISVD (and preferably Nanobody® V_{HH}) against a therapeutic target; or
at least one FcRn binder of the invention and at least one serum albumin binder (as described herein) and at least one ISVD (and preferably Nanobody® V_{HH}) against a therapeutic target; or
at least one FcRn binder of the invention and at least one Fc region of an immunoglobulin (as described herein) and at least one ISVD (and preferably Nanobody® V_{HH}) against a therapeutic target;
or
at least one FcRn binder of the invention and at least one serum albumin binder (as described herein) and at least one Fc region of an immunoglobulin (as described herein) and at least one ISVD (and preferably Nanobody® V_{HH}) against a therapeutic target.

In these polypeptides, compounds or constructs of the invention, the two or more building blocks, ISVDs or Nanobody® V_{HH}s and the optionally one or more polypeptides, one or more other groups, drugs, agents, residues, moieties or binding units may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

For example, a linker may be a suitable amino acid sequence, and in particular amino acid sequences of between 1 and 50, preferably between 1 and 30, such as between 1 and 10 amino acid residues.

Some preferred examples of such amino acid sequences include gly-ser linkers, for example of the type (glyₓser_{y})_{z}, such as (for example (gly₄ser)₃ or (gly₃ser₂)₃, as described in WO 99/42077, and the 30GS, 15GS, 9GS and 7GS linkers described in the applications by Ablynx mentioned herein (see for example WO 06/040153 and WO 06/122825), as well as hinge-like regions, such as the hinge regions of naturally occurring heavy chain antibodies or similar sequences (such as described in WO 94/04678). Preferred linkers are depicted in Table A-2.

Some other particularly preferred linkers are poly-alanine (such as AAA), as well as the linkers GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for FcRn, or for one or more of the other antigens. Based on the disclosure herein, the skilled person will be able to determine the optimal linker(s) for use in a specific polypeptide of the invention, optionally after some limited routine experiments.

For example, in multivalent polypeptides of the invention that comprise building blocks, ISVDs or Nanobody V_{HH}s directed against a first and second target, the length and flexibility of the linker are preferably such that it allows each building block, ISVD or Nanobody V_{HH} of the invention present in the polypeptide to bind to its cognate target, e.g. the antigenic determinant on each of the targets. Again, based on the disclosure herein, the skilled person will be able to determine the optimal linker(s) for use in a specific polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the ISVDs, Nanobody V_{HH}s, or polypeptides of the invention). For example, linkers containing one or more charged amino acid residues can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after some limited routine experiments.

Usually, for ease of expression and production, a polypeptide of the invention will be a linear polypeptide. However, the invention in its broadest sense is not limited thereto. For example, when a polypeptide of the invention comprises three or more building blocks, ISVDs or Nanobody V_{HH}s, it is possible to link them by use of a linker with three or more “arms”, with each “arm” being linked to a building block, ISVD or Nanobody V_{HH}, so as to provide a “star-shaped” construct. It is also possible, although usually less preferred, to use circular constructs.

The invention also relates to methods for preparing the polypeptides, ISVDs, compounds and constructs described herein. The polypeptides, ISVDs, compounds and constructs of the invention can be prepared in a manner known per se, as will be clear to the skilled person from the further description herein. For example, polypeptides, ISVDs, compounds and constructs of the invention can be prepared in any manner known per se for the preparation of antibodies and in particular for the preparation of antibody fragments (including but not limited to (single) domain antibodies and ScFv fragments). Some preferred, but non-limiting methods for preparing the polypeptides and constructs include the methods and techniques described herein.

The methods for producing a polypeptide, ISVD, compound and construct of the invention may comprise the following steps:
the expression, in a suitable host cell or host organism (also referred to herein as a “host of the invention”) or in another suitable expression system of a nucleic acid that encodes said ISVD, polypeptide or protein construct of the invention, optionally followed by:
isolating and/or purifying the polypeptide, ISVD, compound and construct of the invention thus obtained.

In particular, such a method may comprise the steps of:
cultivating and/or maintaining a host cell or host organism of the invention under conditions that are such that said host cell or host organism of the invention expresses and/or produces at least one polypeptide, ISVD, compound and/or construct of the invention; optionally followed by: isolating and/or purifying the polypeptide, ISVD, compound and/or construct of the invention thus obtained.

### 5.2 Nucleic Acid Sequences and Genetic Constructs

Accordingly, the present invention also relates to a nucleic acid or nucleotide sequence that encodes an ISVD, polypeptide, compound, (fusion) protein or (multispecific) construct of the invention (also referred to as “nucleic acid of the invention” or “nucleotide sequence of the invention”). A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein. The nucleic acid of the invention may also be in the form of, be present in and/or be part of a vector, such as for example a plasmid, cosmid or YAC, which again may be in essentially isolated form. A nucleic acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence that is “in essentially isolated form” is preferably essentially homogeneous, as determined using a suitable technique, such as a suitable chromatographical technique, such as polyacrylamide-gel electrophoresis.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

The nucleic acids of the invention can be prepared or obtained in a manner known per se, based on the information on the polypeptides or protein constructs of the invention given herein, and/or can be isolated from a suitable natural source. Also, as will be clear to the skilled person, to prepare a nucleic acid of the invention, also several nucleotide sequences, such as at least one nucleotide sequence encoding an immunoglobulin single variable domain of the invention and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g., to create cassettes and/or regions that may easily be digested and/or ligated using suitable restriction enzymes), and/or the introduction of mutations by means of a PCR reaction using one or more “mismatched” primers. These and other techniques will be clear to the skilled person, and reference is again made to the standard handbooks, such as Sambrook et al. and Ausubel et al., mentioned herein, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises
a) at least one nucleic acid of the invention; operably connected to
b) one or more regulatory elements, such as a promoter and optionally a suitable terminator; and optionally also
c) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used; the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promoter). Generally, when two nucleotide sequences are operably linked, they will be in the same orientation and usually also in the same reading frame. They will usually also be essentially contiguous, although this may also not be required.

### 5.3 Hosts and Host Cells

The nucleic acids of the invention and/or the genetic constructs of the invention may be used to transform a host cell or host organism, i.e. for expression and/or production of the polypeptide or protein construct of the invention. The host is preferably a non-human host. Suitable hosts or host cells will be clear to the skilled person, and may for example be any suitable fungal, prokaryotic or eukaryotic cell or cell line or any suitable fungal, prokaryotic or eukaryotic organism, for example: a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis; a fungal cell, including but not limited to cells from species of Trichoderma, for example from Trichoderma reesei; of Neurospora, for example from Neurosporo crossa; of Sordaria, for example from Sardaria macrospora; of Aspergillus, for example from Aspergillus niger or from Aspergillus sojae; or from other filamentous fungi; a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Honsenula polymorpha; of Kluyveromyces, for example of Kluyveromyces lactis; of Arxula, for example of Arxula adeninivorans; of Yarrowia, for example of Yarrowia lipolytica; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and K_{D} cells; a plant or plant cell, for example in tobacco plants; and/or a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al. 1998 (Res. Immunol. 149: 589-99); Riechmann and Muyldermans 1999 (J. Immunol. Met. 231: 25-38); van der Linden 2000 (J. Biotechnol. 80: 261-70); Joosten et al. 2003 (Microb. Cell Fact. 2: 1); Joosten et aL. 2005 (Appl. Microbiol. Biotechnol. 66: 384-92); and the further references cited herein.

For expression of the polypeptides, ISVDs, compounds or constructs in a cell, they may also be expressed as so-called “intrabodies”, as for example described in WO 94/02610, WO 95/22618 and U.S. Pat. No. 7,004,940; WO 03/014960; in Cattaneo and Biocca 1997 (Intracellular Antibodies: Development and Applications. Landes and Springer-Verlag); and in Kontermann 2004 (Methods 34: 163-170).

According to one preferred, but non-limiting embodiment of the invention, the polypeptide, ISVD, (fusion)protein or construct of the invention is produced in a bacterial cell, in particular a bacterial cell suitable for large scale pharmaceutical production, such as cells of the strains mentioned above.

According to another preferred, but non-limiting embodiment of the invention, polypeptide, ISVD, (fusion)protein, or construct of the invention is produced in a yeast cell, in particular a yeast cell suitable for large scale pharmaceutical production, such as cells of the species mentioned above.

According to yet another preferred, but non-limiting embodiment of the invention, the polypeptide, ISVD, (fusion)protein or construct of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the polypeptide of the invention, e.g.; using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), the ISVD, polypeptide, compound, (fusion)protein or construct of the invention (and in case of a host organism: in at least one cell, part, tissue or organ thereof). The invention also includes further generations, progeny and/or offspring of the host cell or host organism of the invention, for instance obtained by cell division or by sexual or asexual reproduction.

Accordingly, in another aspect, the invention relates to a host or host cell that expresses (or that under suitable circumstances is capable of expressing) an ISVD, polypeptide, (fusion)protein or construct of the invention; and/or that contains a nucleic acid encoding the same. Some preferred but non-limiting examples of such hosts or host cells can be as generally described in WO 04/041867, WO 04/041865 or WO 09/068627. For example, ISVDs, polypeptides, (fusion)proteins and constructs of the invention may with advantage be expressed, produced or manufactured in a yeast strain, such as a strain of Pichia pastoris. Reference is also made to WO 04/25591, WO 10/125187, WO 11/003622, and WO 12/056000 which also describes the expression/production in Pichia and other hosts/host cells of immunoglobulin single variable domains and polypeptides comprising the same.

To produce/obtain expression of the polypeptides, ISVDs, (fusion)proteins or constructs of the invention, the transformed host cell or transformed host organism may generally be kept, maintained and/or cultured under conditions such that the (desired) ISVD, polypeptide, (fusion)protein or construct of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the ISVDs, polypeptides, (fusion)proteins or constructs of the invention may be expressed in a constitutive manner, in a transient manner, or only when suitably induced.

It will also be clear to the skilled person that the polypeptide, ISVD, (fusion)protein or construct of the invention may (first) be generated in an immature form (as mentioned above), which may then be subjected to post-translational modification, depending on the host cell/host organism used. Also, the ISVD, polypeptide, (fusion)protein or construct of the invention may be glycosylated, again depending on the host cell/host organism used.

The polypeptide, ISVD, (fusion)protein or construct of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g., using a specific, cleavable amino acid sequence fused with the polypeptide or construct of the invention) and/or preparative immunological techniques (i.e., using antibodies against the amino acid sequence to be isolated).

An polypeptide or protein is considered to be “(in) essentially isolated (form)”—for example, compared to its native biological source and/or the reaction medium or cultivation medium from which it has been obtained—when it has been separated from at least one other component with which it is usually associated in said source or medium, such as another protein/polypeptide, another biological component or macromolecule or at least one contaminant, impurity or minor component.

In particular, a polypeptide or protein is considered “essentially isolated” when it has been purified at least 2-fold, in particular at least 10-fold, more in particular at least 100-fold, and up to 1000-fold or more. A polypeptide or protein that is “in essentially isolated form” is preferably essentially homogeneous, as determined using a suitable technique, such as a suitable chromatographical technique, such as polyacrylamide-gel electrophoresis.

5.4 Pharmaceutical Compositions, Vaccines and Methods of Treatment and/or Prevention

The invention also relates to a pharmaceutical composition comprising the polypeptide, ISVD compound or construct (multi-specific polypeptide) of the invention. Hence, the present invention also provides the use of the polypeptide, ISVD compound or construct (multi-specific polypeptide) and/or pharmaceutical composition of the invention in medicine (as a medicament). Hence, the present invention provides the polypeptide, ISVD compound or construct (multi-specific polypeptide) and/or pharmaceutical composition of the invention for use in medicine (as a medicament). The invention also relates to the use of the polypeptide, ISVD compound or construct (multi-specific polypeptide) and/or pharmaceutical composition of the invention for therapeutic and/or prophylactic applications (i.e., in a method of therapeutic and/or prophylactic treatment). Hence, the present invention provides the polypeptide, ISVD compound or construct (multi-specific polypeptide) and/or pharmaceutical composition of the invention for use in a method of therapeutic and/or prophylactic treatment (i.e., for therapeutic and/or prophylactic applications).

In the above methods, the polypeptides, ISVDs, compounds or constructs of the invention and/or the compositions comprising the same can be administered in any suitable manner, depending on the specific pharmaceutical formulation or composition to be used. Thus, the polypeptides, ISVDs, compounds or constructs of the invention and/or the compositions comprising the same can for example be administered orally, intraperitoneally, intravenously, subcutaneously, intramuscularly, or via any other route of administration that circumvents the gastrointestinal tract, intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

As used herein, the term “therapeutic agent” refers to any agent that can be used in the prophylaxis (prevention), treatment and/or management of a disease or disorder, such as a hyperproliferative cell disorder, e.g., cancer, or one or more symptoms thereof. In certain embodiments, the term “therapeutic agent” refers to a multispecific polypeptide of the invention. Preferably, a therapeutic agent is an agent which is known to be useful for, or has been or is currently being used for the treatment, prevention and/or management of a disease or disorder, or one or more symptoms thereof.

As used herein, a “therapeutically effective amount” in the present context refers to the amount of a therapy alone, or in combination with other therapies, that provides a therapeutic benefit in the treatment and/or management of a disease and/or disorder. In one aspect, a therapeutically effective amount refers to the amount of a therapy sufficient to cure, modify, stabilize or control a disease and/or disorder, or one or more symptoms thereof. In another aspect, a therapeutically effective amount refers to the amount of a therapy sufficient to reduce the symptoms of a disease and/or disorder. In another aspect, a therapeutically effective amount refers to the amount of a therapy sufficient to delay or minimize the spread of a disease and/or disorder. Used in connection with an amount of a multispecific polypeptide of the invention, the term can encompass an amount that improves overall therapy, reduces or avoids unwanted effects, or enhances the therapeutic efficacy of or synergies with another therapy. In one embodiment, a therapeutically effective amount of a therapy reduces or avoids unwanted effects, or enhances the therapeutic efficacy of or synergies with another therapy by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein.

As used herein, the term “therapy” refers to any protocol, method and/or agent that can be used in the treatment, prevention (prophylaxis) and/or management of a disease and/or disorder, or symptoms thereof. In certain embodiments, the terms “therapies” and “therapy” refer to a biological therapy, supportive therapy, and/or other therapies useful in the treatment, prevention and/or management of a disease and/or disorder, or one or more symptoms thereof known to one of skill in the art, such as medical personnel.

As used herein, the terms “treat”, “treatment” and “treating” in the context of administering (a) therapy(ies) to a subject refer to the reduction or amelioration of the progression, severity, and/or duration of a diseases or disorder, and/or the amelioration of one or more symptoms thereof resulting from the administration of one or more therapies (including, but not limited to, the administration of one or more prophylactic or therapeutic agents). In the context of the present invention, the terms “treat”, “treatment” and “treating” may relate to therapeutic and/or to prophylactic (preventive) treatment. The term “prophylactic treatment” refers to a therapy to reduce the susceptibility to a clinical condition. Thus, the terms “treat”, “treatment”, “treating” and their equivalent terms refer to obtaining a desired pharmacologic or physiologic effect, covering any treatment of a pathological condition, disease or disorder in a mammal, including a human. The effect may be prophylactic in terms of completely or partially preventing pathological condition, disease or disorder or symptom thereof and/or may be therapeutic in terms of a partial or complete cure for a pathological condition, disease or disorder and/or adverse effect attributable to the pathological condition, disease or disorder. That is, “treatment” includes (1) preventing the pathological condition, disease or disorder from occurring or recurring in a subject, (2) inhibiting the pathological condition, disease or disorder, such as arresting its development, (3) stopping or terminating the pathological condition, disease or disorder or, at least, symptoms associated therewith, so that the host no longer suffers from the pathological condition, disease or disorder or its symptoms, such as causing regression of the pathological condition, disease or disorder or its symptoms, or (4) relieving, alleviating, or ameliorating the pathological condition, disease or disorder, or symptoms associated therewith, where ameliorating is used in a broad sense to refer to at least a reduction in the magnitude of a parameter, such as inflammation, pain, or immune deficiency.

The polypeptides, ISVDs, compounds or constructs of the invention and/or the compositions comprising the same are administered according to a regime of treatment that is suitable for preventing and/or treating the disease and/or disorder to be prevented or treated. The clinician will generally be able to determine a suitable treatment regimen, depending on factors such as the stage of the disease and/or disorder to be treated, the severity of the disease and/or disorder to be treated and/or the severity of the symptoms thereof, the specific polypeptide, ISVD, compound or construct of the invention to be used, the specific route of administration and pharmaceutical formulation or composition to be used, the age, gender, weight, diet, general condition of the patient, and similar factors well known to the clinician.

Generally, the treatment regimen will comprise the administration of one or more polypeptides, ISVDs, compounds or constructs of the invention, or of one or more compositions comprising the same, in one or more pharmaceutically effective amounts or doses. The specific amount(s) or doses to be administered can be determined by the clinician, again based on the factors cited above.

Usually, in the above method, a single polypeptide, ISVD, compound or construct of the invention will be used. It is however within the scope of the invention to use two or more polypeptides, ISVDs, compounds and/or constructs of the invention in combination.

The polypeptides, ISVDs, compounds or constructs of the invention may also be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgement.

In particular, the polypeptides, ISVDs, compounds or constructs of the invention may be used in combination with other pharmaceutically active compounds or principles that are or can be used for the prevention and/or treatment of the disease and/or disorder cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g., essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

In one aspect, the disclosure provides methods for the administration of immunoglobulin single variable domains and polypeptide constructs thereof comprising one or more immunoglobulin single variable domains, polypeptides, compounds and/or constructs. In some embodiments, the immunoglobulin single variable domain, polypeptide, compound and/or construct is administered as a pharmaceutical composition. The pharmaceutical composition, in addition to the immunoglobulin single variable domains and polypeptide constructs thereof includes a pharmaceutically acceptable carrier.

Since the compounds or polypeptides of the invention have an increased half-life, they are preferably administered to the circulation. As such, they can be administered in any suitable manner that allows the compound or polypeptide of the invention to enter the circulation, such as intravenously, via injection or infusion, or in any other suitable manner (including oral administration, subcutaneous administration, intramuscular administration, administration through the skin, intranasal administration, administration via the lungs, etc.). Suitable methods and routes of administration will be clear to the skilled person, again for example also from the teaching of the published patent applications of Ablynx N.V., such as for example WO 04/041862, WO 2006/122736, WO 2008/020079, WO 2003/142164 or WO 2009/063627.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Methods of preparing these formulations or compositions include the step of bringing into association an immunoglobulin single variable domain or polypeptide construct with the carrier and, optionally, one or more accessory ingredients. In general, the formulations are prepared by uniformly and intimately bringing into association an immunoglobulin single variable domain or polypeptide construct with liquid carriers, or finely divided solid carriers, or both, and then, if necessary, shaping the product.

The present invention further provides the polypeptide, ISVD compound or construct (multi-specific polypeptide) and/or pharmaceutical composition of the invention as a vaccine, or as an immunogenic composition. Hence, the present invention further provides a vaccine comprising the polypeptide, ISVD compound or construct (multi-specific polypeptide) and/or pharmaceutical composition of the invention.

The term “vaccine”, as used herein, refers to a substance or composition that establishes or improves immunity to a particular disease by inducing an adaptive immune response including an immunological memory, i.e., a substance or composition that, when administered to a subject in an effective amount, stimulates the production of protective antibody or protective T-cell response. Vaccines can be prophylactic or therapeutic. In one aspect, the vaccine of the present invention is a prophylactic vaccine.

The vaccine of the present invention may comprise an “adjuvant”. The term “adjuvant”, as used herein, refers to a substance which, when added to an immunogenic agent, non-specifically enhances or potentiates an immune response to the agent in a recipient host upon exposure to the mixture.

The Figures, Sequence Listing and the Experimental Part/Examples are only given to further illustrate the invention and should not be interpreted or construed as limiting the scope of the invention and/or of the appended claims in any way, unless explicitly indicated otherwise herein.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

The invention will now be further described by means of the following non-limiting preferred aspects, examples and figures.

The entire contents of all of the references (including literature references, issued patents, published patent applications, and co-pending patent applications) cited throughout this application are hereby expressly incorporated by reference, in particular for the teaching that is referenced hereinabove.

### 6. EXAMPLES

### 6.1 Example 1: Selection of FcRn-Specific ISVDs Via Phage Display

The FcRn binding ISVD clones were identified from synthetic libraries. In the synthetic ISVD libraries, sequence diversity (both length and amino acid composition) was introduced into the CDRs of the ISVD sequences. DNA libraries were cloned into a pAX319 vector, pAX319 is an expression vector derived from the commercially available plasmid vector pUC119. In frame with the ISVD protein coding sequence, the vector codes for a C-terminal FLAG3-tag and a His₆-tag. pAX319 allowed for production of phage particles displaying the individual ISVD proteins as a fusion protein with the pill protein. Phages were prepared according to standard protocols (see for example the prior art and applications filed by Ablynx N.V. cited herein) and stored after filter sterilization at 4° C. for further use. The libraries were mined using recombinant biotinylated human FcRn/b2M (hFcRn; Immunitrack ITF01 or ACRO FCM-H82W4) and cyno FcRn/b2M (cFcRn; Immunitrack ITF05 or ACRO FCM-C82W5), alternating between selection rounds. The library phage particles were incubated for 2 h with 0.05 nM-50 nM biotinylated hFcRn or cFcRn in the presence of 1 μM human b2M (Sino Biologicals 11976-H08H) and in the presence or absence of 5 uM human serum albumin (HSA; Albumin Bioscience 9803) and were subsequently captured on streptavidin or neutravidin coated magnetic beads or neutravidin coated plates. The unbound phages were washed away with CPA pH 5.5 or PBS pH 6.0 buffer (supplemented with 0.05% Tween-20 when using beads) and the bound phages were eluted by addition of trypsin (1 mg/ml in PBS), CPA pH 7.4 or PBS pH 7.4 buffer for 5 or 15 min. The trypsin protease activity was immediately neutralized by adding a final concentration of 0.8 mM protease inhibitor AEBSF. As control, selections without antigen were performed in parallel. Eluted phages were used to infect exponentially growing E. coli TG-1 cells for phage rescue. Phages prepared from selected outputs were used as input in subsequent selection rounds. Clones from different selection conditions were individually picked into 96-well master plates for screening. Periplasmic extracts were prepared according to standard protocols (see for example WO 03/035694, WO 04/041865, WO 04/041863, WO 04/062551 and other prior art and applications filed by Ablynx N.V. cited herein).

### 6.2 Example 2: Screening and Sequence Analysis

### 6.2.1 Example 2.1. Binding to Human FcRn/b2M in ELISA

Binding to human FcRn/b2M in ELISA was performed in CPA pH 5.5, PBS pH 6.0 or PBS pH 7.4 (supplemented with 0.05% Tween20 and 0.1% casein). For this, high-binding 384 well Spectraplates (Perkin Elmer) were coated overnight at 4° C. with 2 pg/mL neutravidin and blocked the next day in blocking buffer (PBS+1% casein) at RT for at least one hour. The biotinylated FcRn/b2M (20 nM; human: ACRO (FCM-H82W4)) was captured for one hour at room temperature (RT). Periplasmic extracts containing ISVDs with a Flag₃-His₆ tag were diluted 1:10 or 1:50 in assay buffer supplemented with 0.05% Tween20+0.1% casein and incubated on the coated well at RT for one to two hours. Unbound ISVDs were washed away and bound ISVDs were detected using mouse anti-FLAG-HRP (Sigma; Cat. No. A8592) catalyzing the conversion of chromogenic substrate esTMB (SDT Reagents). Absorbance at 450/620 nm was read on a Tecan Infinite M1000 or Clariostar—BMG (data not shown). ISVD clones which showed binding to human FcRn at acidic pH were sequenced according to commonly known procedures (Pardon et al., 2014) with Sanger sequencing.

### 6.2.2 Example 2.2. Off-Rate Determination Via Surface Plasmon Resonance (SPR)

After sequencing, the unique sequences were re-arrayed and new periplasmic extracts were generated. The FLAG₃-His₆-tagged ISVD proteins were purified by IMAC, using 200 μL Phytips (Phynexus).

All kinetic measurements have been performed on ProteOn XPR36 (BioRad Laboratories, Inc.), MASS-2 (Sierra sensors, Gmbh) or SPR-32 (Bruker Corporation). For off-rate measurements, biotinylated hFcRn/hb2M or cFcRn/cb2M was captured on a ProteOn NLC Sensor (BioRad Laboratories, 1765021) or a MASS-2 Biotin-tag Capture (Bruker Corporation, 1862620) Chip. The phytip purified periplasmic extracts were diluted 1:10 in running buffer (CPA+150 mM NaCl+0.005% Tw20 at pH 5.5, pH 6.0 or pH 7.4) and injected over the ligand surfaces (association: 120 s at 45 μL/min; dissociation: 600s-3600 s at 45 μL/min). Experiments were carried out at 25° C. ISVD clones which showed no detectable off-rate at pH 7.4, or at least a 10-fold difference in off-rate between pH 7.4 and pH 5.5 or pH 6.0 were selected for purification. Obtained off-rates for a selection of clones is shown in Table 1.

TABLE 1
Off-rates of ISVD clones for human and cyno FcRn at pH 5.5 and pH 7.4

| | kd (1/s) cyno FcRn | | | kd (1/s) human FcRn | | |
|---|---|---|---|---|---|---|
| | pH | pH | pH | pH | pH | pH |
| ISVD name | 5.5 | 7.4 | 7.4/5.5 | 5.5 | 7.4 | 7.4/5.5 |
| T0263002A04 | 7.7E−04 | 4.4E−02 | 58 | 5.0E−04 | 3.2E−02 | 64 |
| T0263002A06 | 1.3E−03 | | | 1.6E−03 | | |
| T0263002C10 | 3.8E−03 | | | 8.9E−04 | | |
| T0263002D03 | 1.8E−03 | 8.4E−02 | 47 | 1.5E−03 | 6.7E−02 | 44 |
| T0263002D09 | 3.5E−03 | 3.6E−02 | 10 | 1.6E−03 | 1.8E−02 | 11 |
| T0263002F08 | 9.4E−05 | | | 5.1E−05 | | |
| T0263002F11 | 3.5E−04 | 2.9E−02 | 82 | 1.7E−04 | 2.2E−02 | 129 |
| T0263002G09 | 3.4E−03 | | | 6.9E−04 | | |
| T0263002G10 | 2.5E−03 | | | 1.3E−03 | 2.6E−02 | 20 |
| T0263004A11 | 1.6E−02 | | | 1.5E−02 | | |
| T0263004F02 | 2.3E−02 | | | 2.2E−02 | | |
| T0263005A09 | 2.8E−04 | | | 1.7E−04 | | |
| T0263005A12 | 2.6E−04 | | | 2.6E−04 | | |
| T0263005C03 | 7.6E−03 | | | 7.5E−03 | | |
| T0263005C12 | 9.0E−03 | | | 1.1E−02 | | |
| T0263005D10 | 4.5E−04 | 1.1E−02 | 25 | 3.1E−04 | 5.4E−03 | 17 |
| T0263005E05 | 4.7E−03 | | | 4.9E−03 | | |
| T0263005E06 | 2.9E−04 | 2.7E−02 | 94 | 1.6E−04 | 1.7E−02 | 102 |
| T0263005E10 | 2.5E−03 | 2.9E−02 | 11 | 4.0E−03 | | |
| T0263005F02 | 6.0E−03 | | | 2.9E−03 | | |
| T0263005F06 | 3.6E−03 | | | 2.9E−03 | | |
| T0263005G02 | 2.8E−04 | | | 2.8E−04 | | |
| T0263008E03 | 3.2E−03 | 4.3E−02 | 14 | 1.9E−03 | 1.6E−02 | 8 |
| T0263010G05 | 1.5E−02 | | | 2.6E−02 | | |
| T0263011A09 | 7.5E−03 | | | 7.3E−03 | | |
| T0263011C12 | 5.0E−03 | | | 5.6E−03 | | |
| T0263012A07 | 8.9E−04 | | | 1.0E−03 | | |
| T0263012F05 | 4.4E−04 | | | 4.1E−04 | | |
| T0263017A07 | 2.0E−03 | | | 1.4E−03 | | |
| T0263017C01 | 7.7E−03 | | | 1.1E−02 | | |
| T0263017E07 | 1.3E−04 | | | 1.6E−04 | | |
| T0263017F08 | 1.2E−03 | | | 1.1E−03 | | |
| T0263017G03 | 1.9E−04 | 6.0E−03 | 32 | 4.9E−05 | 1.6E−03 | 33 |
| T0263018A08 | 3.6E−04 | | | 3.2E−04 | | |
| T0263018B11 | 3.3E−04 | 6.5E−02 | 198 | 5.0E−04 | 8.9E−02 | 177 |
| T0263018F03 | 2.0E−03 | 4.8E−02 | 24 | 1.1E−03 | 2.5E−02 | 22 |

Empty cell: no off-rate detected; italics and underlined: indicative values

### 6.3 Example 3: ISVD Production, Purification and Characterization

The ISVDs with pH dependent binding obtained by screening for a more than 10-fold difference in k_{d} (1^{-s}) at pH 7.4 versus pH 5.5 or 6.0 (which also included retaining ISVDs showing specific binding at pH 5.5 or 6.0 but having no detectable off-rate at pH 7.4), were expressed and purified as tagless proteins. A limited number of ISVDs was eventually further selected based on their optimal pH-dependent FcRn binding characteristics (largest off-rate difference between pH 5.5 or 6.0 and 7.4 and highest affinity at pH 5.5 or 6.0) combined with their ability to be optimally expressed and produced at medium to large scale. The selection of ISVDs was characterized by affinity determination for human and cyno FcRn at pH 6.0 and pH 7.4 Additionally, these binders were characterized in an HSA competition FACS to group them based on their possible FcRn epitope (data not shown) and to confirm binding to cell-expressed FcRn at acidic pH.

### 6.3.1 Example 3.1 Generation of ISVD Expression Constructs and Protein Expression

ISVD-containing DNA fragments, obtained by PCR with specific combinations of forward FR1 and reverse FR4 primers each carrying a unique restriction site, were digested with the appropriate restriction enzymes and ligated into the matching cloning cassettes of ISVD expression vectors (as described below). The ligation mixtures were then used to transform electrocompetent or chemically competent Escherichia coli TG1 (Lucigen, Cat. No. 60502 or custom-made, respectively) or TOP10 (ThermoFisher Scientific, Cat. No. C404052 or C4081201, respectively) cells, which were grown under the appropriate antibiotic selection pressure (kanamycin or Zeocin). Resistant clones were verified by Sanger sequencing of plasmid DNA (LGC Genomics).

Monovalent ISVDs were expressed in E. coli TG1 cells (Lucigen, Cat. No. 60502). Reference is also made to WO 04/25591, WO 10/125187, WO 11/003622, and WO 12/056000 which also describe the expression/production in E. coli and other hosts/host cells of immunoglobulin single variable domains and polypeptides comprising the same.

Multivalent ISVD constructs were expressed in P. pastoris. The yeast expression vectors contain the AOX1 promoter and terminator, a resistance gene for Zeocin and the coding information for the Saccharomyces cerevisiae a-mating factor signal peptide. The ISVD building blocks were combined with GS linkers and cloned in the expression vector via Golden Gate cloning (Engler C, Marillonnet S. Golden Gate cloning. Methods Mol Biol. 2014; 1116:119-31). The expression vectors contain two Bpil restriction sites for the cloning of the PCR-amplified monovalent ISVDs together with the GS linkers included in one or multiple vectors. All these elements are flanked by Bpil sites. The use of unique nucleotide overhangs for each position of the cloning cassette allows seamless ligation in a pre-defined order. After Sanger sequence confirmation, plasmid DNA derived from E. coli TOP10 was linearized and transformed by electroporation into in-house prepared hypercompetent P. pastoris, strain NRRL Y-11430 (ATCC 76273).

### 6.3.2 Example 3.2 ISVD Purification

His₆-tagged ISVDs were purified by methods known in the art, and tagless ISVDs were purified with protein A chromatography as known in the art, and concentration was determined via OD280/OD340 measurement. Quality control was performed by SDS-PAGE and mass spectrometry.

### 6.3.3 Example 3.3 Affinity Determination Via Surface Plasmon Resonance (SPR) of Monovalent and Bivalent ISVDs

The selected ISVDs were characterized by affinity determination for human FcRn at pH 6.0 and pH 7.4.

The affinities of the monovalent purified, tagless ISVDs were determined for human FcRn on ProteOn XPR36 (BioRad Laboratories, Inc.), the MASS-2 (Sierra sensors, Gmbh) or SPR-32 (Bruker Corporation) instrument. In the same way, the affinities of bivalent ISVD constructs comprising two of the same monovalent building blocks of purified, tagless ISVDs were determined for human FcRn on ProteOn XPR36 (BioRad Laboratories, Inc.), the MASS-2 (Sierra sensors, Gmbh) or SPR-32 (Bruker Corporation) instrument. For affinity measurements, ˜1000-3000RU (hFcRn/hb2M; ACRO Biosystems FCM-H82W4) was captured on a ProteOn NLC sensor (ProteOn) or Biotin-tag Capture Chip or a High Capacity Amine Chip (pre-immobilized with neutravidin; MASS-2). The ISVDs were injected at 6 different concentrations (between 1 and 4000 nM) and allowed to associate for 120s at 45 μL/min and dissociate for 600s-3600s at 45 μL/min. Higher ISVD concentrations were used to determine the affinity at pH 7.4 compared to pH 5.5 or 6.0, due to the lower binding at pH 7.4. Evaluation of the sensorgrams was based on the 1:1 Langmuir dissociation model simultaneously fitting on and off-rates. For some selected ISVDs, the affinity for human FcRn at pH 6.0 and pH 7.4 is shown in Table 2.

TABLE 2
Affinities of monovalent and bivalent ISVDs
for human FcRn at pH 6.0 and pH 7.4

| | | K_{D} (M) human FcRn | | |
|---|---|---|---|---|
| ISVD name | Format | pH 6.0 | pH 7.4 | pH 7.4/6.0 |
| T0263002A06 | Monovalent | 2.5E−08 | 1.6E−06 | 62 |
| | Bivalent | 2.1E−08 | 1.1E−06 | 50 |
| T0263002F11 | Monovalent | 7.9E−10 | 4.7E−08 | 60 |
| | Bivalent | 1.5E−09 | 9.6E−08 | 66 |
| T0263018B11 | Monovalent | 8.3E−09 | 5.4E−07 | 65 |
| | Bivalent | 1.9E−08 | 1.0E−06 | 54 |
| T0263012A07 | Monovalent | 3.3E−08 | 1.1E−05 | 323 |
| | Bivalent | 7.9E−08 | 3.8E−06 | 48 |
| T0263018A08 | Monovalent | 2.5E−08 | 4.9E−06 | 198 |

### 6.4 Example 4: Engineering of T0263018B11

The ELISA was performed as described in Example 2.1 in PBS at pH 6.0 (dilution periplasmic extracts is 1:1000) and pH 7.4 (dilution periplasmic extracts is 1:10). After screening and sequencing, phytip purified periplasmic extracts were produced for 206 unique clones (containing a FLAG3-His6 tag) and the off-rate of these clones on human and cyno FcRn at pH 6.0 and pH 7.4, were determined as described in Example 2.2 (see Table 3). Sixteen substitutions with a larger fold-difference in kd at pH 7.4 versus pH 6.0 compared to the parental T0263018B11 were selected for subcloning and purification as tagless ISVD proteins (as described in Example 3.1 and 3.2) and characterized by affinity determination (see Table 4).

TABLE 3
Off-rates of T0263018B11 single substitution variants
for human and cyno FcRn at pH 6.0 and pH 7.4

| | humanFcRn | | | cynoFcRn | | |
|---|---|---|---|---|---|---|
| Substitution | pH 6.0 | pH 7.4 | pH 7.4/6.0 | pH 6.0 | pH 7.4 | pH 7.4/6.0 |
| (Kabat) | k_{d} (s⁻¹) | k_{d} (s⁻¹) | Ratio | k_{d} (s⁻¹) | k_{d} (s⁻¹) | ratio |
| Parental | 8.8E−04 | 2.8E−02 | 32 | 6.5E−04 | 2.5E−02 | 38 |
| G26D | 1.2E−03 | 6.3E−02 | 53 | 9.5E−04 | 5.8E−02 | 61 |
| G26E | 9.5E−04 | 4.2E−02 | 44 | 8.1E−04 | 5.0E−02 | 62 |
| G26F | 7.8E−04 | 3.1E−02 | 40 | 6.6E−04 | 3.6E−02 | 55 |
| G26L | 1.0E−03 | 4.1E−02 | 41 | 8.7E−04 | 5.4E−02 | 62 |
| G26S | 9.1E−04 | 4.7E−02 | 52 | 7.9E−04 | 5.3E−02 | 67 |
| G26T | 1.8E−03 | 1.3E−01 | 72 | 1.5E−03 | 1.8E−01 | 120 |
| G26Y | 6.5E−04 | 2.7E−02 | 42 | 5.5E−04 | 2.8E−02 | 51 |
| G26A | 6.8E−04 | 3.7E−02 | 55 | 2.4E−04 | 2.8E−02 | 116 |
| G26H | 5.5E−04 | 3.4E−02 | 62 | 1.6E−04 | 2.6E−02 | 159 |
| G26K | 6.1E−04 | 3.4E−02 | 55 | 2.5E−04 | 2.5E−02 | 101 |
| G26M | 6.3E−04 | 3.3E−02 | 52 | 7.1E−05 | 2.5E−02 | 349 |
| G26N | 8.1E−04 | 4.2E−02 | 53 | 3.7E−04 | 3.2E−02 | 86 |
| G26Q | 6.5E−04 | 3.4E−02 | 53 | 1.3E−05 | 2.6E−02 | 2008 |
| G26W | 5.0E−04 | 2.7E−02 | 55 | ND | ND | ND |
| F27L | 1.3E−03 | 5.0E−02 | 38 | 1.0E−03 | 4.8E−02 | 48 |
| F27V | 1.9E−03 | 9.6E−02 | 51 | 1.3E−03 | 7.7E−02 | 59 |
| F27I | 1.0E−03 | 4.5E−02 | 45 | 3.6E−04 | 3.1E−02 | 85 |
| F27M | 8.3E−04 | 3.9E−02 | 47 | 4.0E−04 | 2.8E−02 | 69 |
| F27Y | 1.2E−03 | 5.4E−02 | 45 | 4.2E−04 | 3.9E−02 | 93 |
| T28E | 2.8E−03 | 1.4E−01 | 50 | 1.9E−03 | 1.3E−01 | 68 |
| T28L | 1.8E−03 | 7.3E−02 | 41 | 1.3E−03 | 6.2E−02 | 48 |
| T28P | 1.5E−03 | 3.9E−02 | 26 | 1.2E−03 | 3.3E−02 | 28 |
| T28A | 1.0E−03 | 4.6E−02 | 44 | 5.2E−04 | 3.3E−02 | 65 |
| T28G | 7.5E−04 | 3.8E−02 | 51 | 2.0E−04 | 2.8E−02 | 138 |
| T28I | 1.1E−03 | 4.9E−02 | 46 | 4.4E−04 | 3.3E−02 | 75 |
| T28M | 7.2E−04 | 3.3E−02 | 47 | 3.7E−04 | 2.4E−02 | 64 |
| T28N | 1.9E−03 | 9.7E−02 | 51 | 9.0E−04 | 6.9E−02 | 77 |
| T28S | 6.1E−04 | 3.0E−02 | 48 | 2.5E−04 | 2.1E−02 | 85 |
| T28W | 4.1E−04 | 2.4E−02 | 59 | 1.7E−04 | 1.4E−02 | 80 |
| T28Y | 1.2E−03 | 7.1E−02 | 59 | 5.3E−04 | 4.2E−02 | 79 |
| T28V | 3.2E−03 | 1.2E−01 | 38 | 2.2E−03 | 9.2E−02 | 42 |
| F29D | 3.6E−03 | 1.4E−01 | 39 | 2.8E−03 | 1.5E−01 | 54 |
| F29G | 3.3E−03 | 1.5E−01 | 45 | 2.5E−03 | 1.4E−01 | 56 |
| F29H | 3.9E−03 | 1.4E−01 | 36 | 3.1E−03 | 1.3E−01 | 42 |
| F29I | 2.0E−03 | 1.0E−01 | 50 | 1.5E−03 | 8.3E−02 | 55 |
| F29L | 2.3E−03 | 1.2E−01 | 52 | 1.7E−03 | 1.1E−01 | 65 |
| F29P | 2.6E−03 | 1.1E−01 | 42 | 1.9E−03 | 1.1E−01 | 58 |
| F29Q | 2.1E−03 | 9.3E−02 | 44 | 1.7E−03 | 8.8E−02 | 52 |
| F29S | 1.6E−03 | 6.6E−02 | 41 | 1.3E−03 | 5.9E−02 | 45 |
| F29T | 2.1E−03 | 1.0E−01 | 48 | 1.6E−03 | 8.6E−02 | 54 |
| F29V | 2.1E−03 | 8.9E−02 | 42 | 1.6E−03 | 8.6E−02 | 54 |
| F29Y | 1.5E−03 | 6.0E−02 | 40 | 1.2E−03 | 5.4E−02 | 45 |
| S30D | 3.2E−03 | 1.3E−01 | 41 | 2.6E−03 | 1.7E−01 | 65 |
| S30E | 1.9E−03 | 7.1E−02 | 37 | 1.6E−03 | 7.3E−02 | 46 |
| S30T | 1.6E−03 | 5.9E−02 | 37 | 1.4E−03 | 6.2E−02 | 44 |
| S30G | 6.6E−04 | 2.5E−02 | 38 | 5.4E−04 | 1.9E−02 | 35 |
| S30K | 7.8E−03 | 6.9E−02 | 9 | 3.0E−03 | 5.5E−02 | 18 |
| S30M | 7.4E−04 | 3.1E−02 | 42 | 6.9E−04 | 2.2E−02 | 32 |
| S30N | 1.9E−03 | 8.2E−02 | 42 | 1.3E−03 | 6.1E−02 | 46 |
| S30P | 6.1E−04 | 2.5E−02 | 40 | 4.3E−04 | 1.8E−02 | 41 |
| S30Q | 8.5E−04 | 3.5E−02 | 42 | 5.5E−04 | 2.6E−02 | 47 |
| S31E | 1.5E−03 | 1.0E−01 | 67 | 1.2E−03 | 1.0E−01 | 83 |
| S31G | 1.4E−03 | 6.7E−02 | 48 | 1.1E−03 | 6.4E−02 | 58 |
| S31K | 1.3E−03 | 4.8E−02 | 37 | 1.1E−03 | 4.4E−02 | 40 |
| S31P | 8.8E−04 | 4.3E−02 | 49 | 7.3E−04 | 4.3E−02 | 59 |
| S31A | 4.1E−04 | 2.0E−02 | 47 | 2.3E−04 | 1.4E−02 | 61 |
| S31N | 3.4E−03 | 1.7E−01 | 50 | 1.8E−03 | 1.2E−01 | 69 |
| S31Q | 3.8E−04 | 1.7E−02 | 43 | 1.6E−04 | 1.3E−02 | 79 |
| S31T | 5.2E−04 | 2.4E−02 | 46 | 3.0E−04 | 1.8E−02 | 60 |
| S31V | 2.9E−04 | 1.3E−02 | 47 | 1.1E−04 | 9.5E−03 | 89 |
| Y32A | 2.7E−03 | 1.3E−01 | 48 | 2.3E−03 | 1.6E−01 | 70 |
| Y32T | 4.5E−03 | 1.9E−01 | 42 | 3.5E−03 | 2.1E−01 | 60 |
| Y32F | 6.6E−04 | 2.6E−02 | 39 | 4.2E−04 | 1.9E−02 | 46 |
| Y32L | 1.3E−02 | 6.1E−01 | 49 | 8.4E−03 | 3.9E−01 | 46 |
| Y32M | 6.0E−03 | 2.3E−01 | 39 | 2.5E−03 | 1.8E−01 | 72 |
| Y32N | 2.5E−03 | 1.1E−01 | 42 | 1.8E−03 | 8.3E−02 | 47 |
| Y32Q | 2.3E−03 | 1.4E−01 | 60 | 1.3E−03 | 9.9E−02 | 75 |
| Y32S | 5.4E−03 | 1.9E−01 | 35 | 2.8E−03 | 1.4E−01 | 52 |
| Y32V | 4.0E−03 | 4.8E−02 | 12 | 2.2E−03 | 1.3E−01 | 60 |
| A33D | 1.4E−03 | 1.8E−01 | 129 | 1.8E−03 | 3.4E−01 | 189 |
| A33E | 3.2E−03 | 1.9E−01 | 59 | 5.1E−03 | 5.0E−01 | 98 |
| A33G | 1.3E−03 | 3.4E−02 | 26 | 1.1E−03 | 4.0E−02 | 36 |
| A33P | 1.0E−03 | 4.1E−02 | 41 | 9.9E−04 | 4.5E−02 | 45 |
| A33Q | 1.8E−03 | 6.5E−02 | 36 | 2.7E−03 | 1.3E−01 | 48 |
| A33S | 1.5E−03 | 5.9E−02 | 39 | 1.3E−03 | 6.5E−02 | 50 |
| A33H | 7.6E−04 | 3.1E−02 | 41 | 7.7E−04 | 3.7E−02 | 48 |
| A33M | 2.3E−03 | 1.1E−01 | 48 | 2.1E−03 | 1.5E−01 | 71 |
| A33N | 1.6E−03 | 6.5E−02 | 40 | 1.6E−03 | 7.7E−02 | 49 |
| A33T | 1.1E−04 | 3.8E−03 | 36 | 1.1E−04 | 5.8E−03 | 54 |
| A33V | 2.2E−04 | 8.2E−03 | 37 | 2.4E−04 | 1.3E−02 | 53 |
| Y35F | 4.3E−03 | 1.4E−01 | 33 | 3.5E−03 | 1.6E−01 | 46 |
| S52A | 1.1E−03 | 3.1E−02 | 28 | 9.8E−04 | 3.6E−02 | 37 |
| S52G | 3.9E−04 | 1.2E−02 | 32 | 4.0E−04 | 1.1E−02 | 27 |
| S52H | 3.2E−03 | 1.5E−01 | 46 | 2.2E−03 | 1.1E−01 | 48 |
| S52Q | 6.7E−03 | 1.5E−01 | 22 | 5.5E−03 | 1.4E−01 | 26 |
| S52R | 2.3E−02 | 4.4E−01 | 20 | 1.6E−02 | 3.8E−01 | 24 |
| S52T | 1.9E−03 | 4.5E−02 | 24 | 1.7E−03 | 4.2E−02 | 25 |
| S52V | 2.4E−03 | 5.8E−02 | 24 | 2.5E−03 | 6.4E−02 | 25 |
| S52aD | 1.0E−03 | 4.4E−02 | 44 | 8.9E−04 | 5.1E−02 | 57 |
| S52aE | 8.7E−04 | 3.6E−02 | 41 | 7.7E−04 | 3.9E−02 | 51 |
| S52aK | 5.2E−03 | 3.9E−01 | 75 | 3.5E−03 | 3.9E−01 | 111 |
| S52aQ | 1.5E−03 | 7.0E−02 | 47 | 1.1E−03 | 6.8E−02 | 62 |
| S52aT | 1.6E−03 | 6.2E−02 | 39 | 1.2E−03 | 5.9E−02 | 49 |
| S52aV | 2.7E−03 | 8.1E−02 | 30 | 2.1E−03 | 9.5E−02 | 45 |
| S52aA | 6.8E−04 | 2.9E−02 | 43 | 4.3E−04 | 2.2E−02 | 51 |
| S52aG | 8.8E−04 | 3.4E−02 | 39 | 6.0E−04 | 3.1E−02 | 51 |
| S52aM | 4.8E−04 | 1.8E−02 | 36 | 2.7E−04 | 1.3E−02 | 48 |
| S52aN | 1.2E−03 | 6.3E−02 | 51 | 7.9E−04 | 4.7E−02 | 60 |
| S52aP | 5.4E−04 | 2.9E−02 | 53 | 3.0E−04 | 2.1E−02 | 71 |
| S52aR | 2.3E−03 | 1.3E−01 | 56 | 1.5E−03 | 9.1E−02 | 61 |
| S52aW | 1.5E−03 | 6.2E−02 | 41 | 8.0E−04 | 4.3E−02 | 54 |
| S52aY | 8.3E−04 | 3.5E−02 | 42 | 5.2E−04 | 2.3E−02 | 44 |
| S52D | 4.4E−03 | 1.4E−01 | 32 | 3.1E−03 | 1.9E−01 | 61 |
| S52E | 2.6E−03 | 4.2E−02 | 16 | 2.3E−03 | 4.7E−02 | 20 |
| S52P | 1.1E−03 | 3.0E−02 | 27 | 1.3E−03 | 4.8E−02 | 37 |
| G53E | 1.5E−03 | 5.7E−02 | 38 | 1.1E−03 | 5.5E−02 | 50 |
| G53P | 1.1E−03 | 3.1E−02 | 28 | 9.5E−04 | 3.2E−02 | 34 |
| G54D | 2.2E−03 | 6.2E−02 | 28 | 2.2E−03 | 9.0E−02 | 41 |
| G54E | 1.6E−03 | 4.6E−02 | 29 | 1.6E−03 | 6.2E−02 | 39 |
| G54H | 1.2E−03 | 3.8E−02 | 32 | 1.3E−03 | 5.0E−02 | 38 |
| G54L | 1.6E−03 | 4.1E−02 | 26 | 1.7E−03 | 6.0E−02 | 35 |
| G54P | 1.6E−03 | 3.3E−02 | 21 | 1.7E−03 | 4.1E−02 | 24 |
| G54T | 1.6E−03 | 4.5E−02 | 28 | 1.7E−03 | 6.0E−02 | 35 |
| G54V | 1.6E−03 | 3.6E−02 | 23 | 1.7E−03 | 5.2E−02 | 31 |
| G55D | 1.7E−03 | 7.0E−02 | 41 | 1.3E−03 | 7.0E−02 | 54 |
| G55E | 1.9E−03 | 6.6E−02 | 35 | 1.4E−03 | 6.7E−02 | 48 |
| S56A | 1.3E−03 | 5.1E−02 | 39 | 1.0E−03 | 4.5E−02 | 45 |
| S56E | 2.6E−03 | 1.1E−01 | 42 | 1.9E−03 | 1.0E−01 | 53 |
| S56Q | 2.2E−03 | 6.7E−02 | 30 | 1.8E−03 | 6.8E−02 | 38 |
| S56G | 4.7E−04 | 1.9E−02 | 40 | 2.7E−04 | 1.4E−02 | 54 |
| S56H | 1.6E−03 | 5.5E−02 | 35 | 1.3E−03 | 4.6E−02 | 36 |
| S56K | 2.1E−03 | 5.3E−02 | 25 | 1.8E−03 | 4.4E−02 | 25 |
| S56L | 7.5E−04 | 2.0E−02 | 26 | 4.5E−04 | 1.3E−02 | 28 |
| S56M | 1.1E−03 | 3.2E−02 | 31 | 7.8E−04 | 2.3E−02 | 30 |
| S56T | 1.3E−03 | 5.5E−02 | 42 | 1.0E−03 | 4.7E−02 | 47 |
| S56V | 1.3E−03 | 4.4E−02 | 34 | 1.0E−03 | 3.8E−02 | 37 |
| T57F | 9.9E−04 | 3.3E−02 | 33 | 8.4E−04 | 3.6E−02 | 43 |
| T57G | 1.3E−03 | 3.8E−02 | 29 | 1.1E−03 | 4.0E−02 | 36 |
| T57I | 1.5E−03 | 4.9E−02 | 33 | 1.4E−03 | 6.0E−02 | 43 |
| T57P | 1.5E−03 | 4.5E−02 | 30 | 1.3E−03 | 5.4E−02 | 42 |
| T57Y | 1.0E−03 | 3.3E−02 | 33 | 8.4E−04 | 3.5E−02 | 42 |
| T57A | 9.3E−04 | 3.7E−02 | 40 | 6.4E−04 | 2.3E−02 | 36 |
| T57D | 7.3E−04 | 3.1E−02 | 43 | 6.3E−04 | 2.6E−02 | 41 |
| T57E | 7.6E−04 | 3.1E−02 | 41 | 5.5E−04 | 2.4E−02 | 43 |
| T57H | 8.4E−04 | 3.3E−02 | 40 | 5.9E−04 | 2.4E−02 | 41 |
| T57K | 8.2E−04 | 3.3E−02 | 40 | 5.8E−04 | 2.3E−02 | 40 |
| T57L | 1.1E−03 | 4.0E−02 | 37 | 9.0E−04 | 3.5E−02 | 39 |
| T57M | 9.8E−04 | 3.8E−02 | 39 | 7.0E−04 | 2.7E−02 | 39 |
| T57N | 8.6E−04 | 3.7E−02 | 43 | 6.2E−04 | 3.0E−02 | 48 |
| T57Q | 7.4E−04 | 3.0E−02 | 40 | 5.6E−04 | 1.9E−02 | 34 |
| T57R | 8.6E−04 | 3.8E−02 | 44 | 7.1E−04 | 2.6E−02 | 37 |
| T57S | 1.1E−03 | 4.1E−02 | 39 | 7.1E−04 | 2.9E−02 | 41 |
| T57V | 7.7E−04 | 3.4E−02 | 44 | 6.4E−04 | 2.7E−02 | 42 |
| T57W | 9.0E−04 | 3.3E−02 | 37 | 7.3E−04 | 2.8E−02 | 38 |
| D58E | 2.4E−03 | 8.0E−02 | 33 | 1.2E−03 | 3.8E−02 | 32 |
| D58A | 8.9E−03 | 8.4E−02 | 9 | 2.6E−03 | 3.6E−02 | 14 |
| D58H | 1.2E−02 | 7.9E−02 | 7 | 2.0E−03 | 2.2E−02 | 11 |
| D58S | 9.9E−03 | 9.8E−02 | 10 | 4.0E−03 | 4.7E−02 | 12 |
| D58T | 1.8E−02 | 1.8E−01 | 10 | 5.8E−03 | 6.0E−02 | 10 |
| D58V | 1.5E−02 | 1.4E−01 | 9 | 2.5E−03 | 2.7E−02 | 11 |
| D58Y | 1.0E−02 | 8.9E−02 | 9 | 2.0E−03 | 2.5E−02 | 12 |
| A93T | 2.5E−03 | 1.1E−01 | 44 | 1.9E−03 | 1.2E−01 | 63 |
| A93V | 3.0E−03 | 1.5E−01 | 50 | 2.1E−03 | 1.4E−01 | 67 |
| A94S | 1.3E−03 | 4.6E−02 | 35 | 1.1E−03 | 4.7E−02 | 43 |
| T96A | 3.9E−03 | 1.4E−01 | 36 | 2.9E−03 | 1.6E−01 | 55 |
| T96F | 3.1E−03 | 1.6E−01 | 52 | 2.4E−03 | 2.0E−01 | 83 |
| T96L | 1.2E−03 | 4.6E−02 | 38 | 1.1E−03 | 5.7E−02 | 52 |
| T96Q | 2.6E−03 | 9.1E−02 | 35 | 2.0E−03 | 9.2E−02 | 46 |
| T96S | 1.6E−03 | 5.3E−02 | 33 | 1.2E−03 | 5.3E−02 | 44 |
| T96E | 1.2E−02 | No binding | ND | 9.3E−03 | No binding | ND |
| T96G | 1.4E−02 | 2.9E−01 | 21 | 8.4E−03 | 2.1E−01 | 25 |
| T96I | 6.9E−04 | 3.6E−02 | 52 | 4.2E−04 | 2.4E−02 | 57 |
| T96M | 1.6E−03 | 6.7E−02 | 42 | 9.7E−04 | 4.7E−02 | 49 |
| T96V | 4.7E−04 | 2.1E−02 | 45 | 3.6E−04 | 1.4E−02 | 40 |
| T96W | 2.4E−03 | 9.6E−02 | 40 | 1.3E−03 | 7.0E−02 | 54 |
| T96Y | 5.4E−04 | 2.4E−02 | 45 | 3.5E−04 | 1.7E−02 | 50 |
| Y98F | 5.3E−04 | 4.0E−02 | 75 | 2.6E−04 | 2.2E−02 | 84 |
| Y98L | 1.4E−03 | 4.0E−02 | 28 | 8.6E−04 | 3.1E−02 | 36 |
| Y98W | 2.2E−03 | 1.2E−01 | 55 | 1.3E−03 | 5.8E−02 | 45 |
| S100D | 4.4E−03 | 2.8E−01 | 64 | 2.0E−03 | 1.4E−01 | 70 |
| S100A | 2.5E−04 | 1.1E−02 | 45 | 2.1E−04 | 9.9E−03 | 48 |
| S100E | 3.1E−03 | No binding | ND | 1.3E−03 | 3.2E−01 | 253 |
| S100K | 3.2E−03 | 1.8E−01 | 54 | 1.2E−03 | 8.3E−02 | 69 |
| S100L | 7.8E−04 | 4.3E−02 | 55 | 4.5E−04 | 2.8E−02 | 62 |
| S100M | 5.6E−04 | 3.2E−02 | 57 | 2.4E−04 | 1.6E−02 | 68 |
| S100Q | 1.9E−03 | 1.3E−01 | 68 | 8.4E−04 | 5.8E−02 | 69 |
| S100W | 6.2E−04 | 3.1E−02 | 49 | 2.9E−04 | 1.5E−02 | 51 |
| L100aM | 1.5E−02 | 6.2E−01 | 42 | 1.3E−02 | 5.1E−01 | 41 |
| L100aV | 9.3E−04 | 3.7E−02 | 40 | 7.0E−04 | 2.7E−02 | 39 |
| T100bE | 7.1E−04 | 3.2E−02 | 45 | 4.9E−04 | 2.5E−02 | 51 |
| T100bL | 3.7E−03 | 1.4E−01 | 38 | 3.2E−03 | 1.8E−01 | 56 |
| W100cA | 1.0E−02 | 5.0E−01 | 50 | 6.2E−03 | 5.0E−01 | 81 |
| W100cE | 7.5E−03 | 3.5E−01 | 47 | 4.2E−03 | 3.0E−01 | 71 |
| W100cF | 3.6E−03 | 1.6E−01 | 44 | 2.4E−03 | 1.5E−01 | 63 |
| W100cG | 9.2E−03 | 4.2E−01 | 46 | 5.1E−03 | 3.8E−01 | 75 |
| W100cH | 1.0E−02 | 2.9E−01 | 29 | 7.1E−03 | 2.8E−01 | 39 |
| W100cK | 4.6E−03 | 4.6E−01 | 100 | 5.0E−03 | 3.5E−01 | 70 |
| W100cL | 6.4E−03 | 4.6E−01 | 72 | 5.1E−03 | 4.3E−01 | 84 |
| W100cQ | 9.0E−03 | 5.0E−01 | 56 | 5.9E−03 | 5.0E−01 | 85 |
| W100cS | 3.7E−03 | 2.1E−01 | 57 | 2.2E−03 | 1.6E−01 | 73 |
| W100cT | 3.5E−03 | 1.8E−01 | 51 | 2.3E−03 | 1.5E−01 | 65 |
| W100cY | 2.1E−03 | 9.0E−02 | 43 | 1.5E−03 | 7.5E−02 | 50 |
| W100cD | 7.3E−03 | 1.4E−01 | 19 | 3.0E−03 | 1.3E−01 | 45 |
| W100cM | 9.6E−03 | 5.3E−01 | 55 | 1.8E−03 | 1.1E−01 | 59 |
| W100cR | 8.0E−03 | 3.1E−01 | 39 | 1.4E−03 | 4.6E−02 | 33 |
| W100cV | 8.2E−03 | 5.0E−01 | 61 | 2.4E−03 | 1.8E−01 | 76 |
| Y100dF | 2.3E−03 | 6.0E−02 | 26 | 1.7E−03 | 5.7E−02 | 34 |
| S101A | 1.7E−03 | 6.2E−02 | 36 | 1.3E−03 | 5.8E−02 | 45 |
| S101E | 1.8E−04 | 7.8E−03 | 44 | 9.4E−05 | 3.4E−03 | 36 |
| S101L | 2.5E−03 | 1.0E−01 | 40 | 1.5E−03 | 7.7E−02 | 52 |
| S101M | 1.1E−03 | 4.8E−02 | 44 | 6.8E−04 | 3.7E−02 | 54 |
| S101Q | 5.5E−04 | 2.5E−02 | 45 | 3.8E−04 | 1.6E−02 | 43 |
| S101T | 5.7E−04 | 2.5E−02 | 44 | 3.6E−04 | 1.9E−02 | 54 |
| S101V | 7.3E−04 | 3.2E−02 | 43 | 5.8E−04 | 2.6E−02 | 45 |
| S101W | 7.0E−05 | 1.2E−03 | 17 | 9.7E−05 | 8.6E−04 | 9 |
| Y102T | 1.7E−03 | 6.5E−02 | 38 | 1.2E−03 | 6.0E−02 | 50 |

ND: not determined

TABLE 4
Affinity (KD values) of T0263018B11 single substitution
variants for human and cyno FcRn at pH 6.0 and pH 7.4

| | | | | | | | | Selected |
|---|---|---|---|---|---|---|---|---|
| | | human FcRn | | | Cyno FcRn | | | for |
| | | pH | pH | pH | pH | pH | pH | formatting |
| | Substitution | 6.0 | 7.4 | 7.4/6.0 | 6.0 | 7.4 | 7.4/6.0 | and in vivo |
| ISVD name | (Kabat) | K_{D} (M) | K_{D} (M) | Ratio | K_{D} (M) | K_{D} (M) | Ratio | testing |
| T0263018B11 | Parental | 7.3E−09 | 6.0E−07 | 82 | 7.2E−09 | 4.3E−07 | 60 | |
| T026301028 | T96L | 1.5E−08 | 1.1E−06 | 73 | 1.1E−08 | 7.5E−07 | 68 | |
| T026301029 | S52aK | 7.4E−08 | 7.2E−06 | 97 | 4.9E−08 | 5.1E−06 | 104 | |
| T026301030 | S31E | 1.9E−08 | 2.5E−06 | 132 | 1.4E−08 | 1.0E−06 | 71 | |
| T026301031 | A33E | 3.3E−08 | 7.0E−06 | 212 | 5.0E−08 | 3.5E−05 | 700 | |
| T026301032 | F29L | 2.1E−08 | 2.1E−06 | 100 | 1.6E−08 | 1.4E−06 | 88 | |
| T026301033 | S100D | 9.0E−08 | 4.7E−05 | 522 | 6.7E−08 | ND | NA | |
| T026301034 | W100cT | 6.4E−08 | 5.8E−06 | 91 | 4.4E−08 | 2.9E−06 | 66 | |
| T026301035 | G55E | 2.1E−08 | 1.8E−06 | 86 | 1.9E−08 | 1.3E−06 | 68 | |
| T026301036 | G26T | 2.1E−08 | 3.0E−06 | 143 | 1.9E−08 | 2.3E−06 | 121 | |
| T026301037 | W100cK | 3.1E−07 | ND | NA | 1.7E−07 | ND | NA | |
| T026301038 | S52aQ | 1.5E−08 | 1.7E−06 | 113 | 1.2E−08 | 8.6E−07 | 72 | Yes |
| T026301039 | W100cS | 4.9E−08 | 6.1E−06 | 125 | 3.3E−08 | 1.2E−06 | 36 | Yes |
| T026301040 | A33D | 1.7E−08 | 5.6E−06 | 329 | 3.3E−08 | 2.8E−05 | 848 | |
| T026301041 | F27V | 1.9E−08 | 1.8E−06 | 95 | 1.6E−08 | 1.1E−06 | 69 | |
| T026301042 | T28E | 3.6E−08 | 3.2E−06 | 89 | 3.3E−08 | 2.1E−06 | 64 | |
| T026301043 | G53E | 1.5E−08 | 1.3E−06 | 87 | 1.6E−08 | 8.6E−07 | 54 | |

ND: not determined
NA: not available

Based on the affinity data, a set of tagless ISVD combinatorial variants was generated that contain all possible combinations of the mutations G26T, S31E, A33D, S52aQ, S100D, W100cS and W100cK, including the parental amino acid for all 6 positions. In addition, some additional single variants on the same Kabat positions were explored, i.e., S31D, S100E and W100cR. Periplasmic extracts containing these tagless ISVD variants were produced and the off-rates were determined at pH 6.0 (see Table 5). Nineteen variants were selected for purification and further characterization by affinity determination at pH 6.0 and pH 7.4 (see Table 6) and TSA (not shown). Based on the affinity data, 5 variants of the FcRn ISVD T0263018B11 (including single and combinatorial variants) were selected for formatting and in vivo characterization (see Examples 8 and 10).

TABLE 5
Off-rate analysis of combinatorial variants
of T0263018B11 on human FcRn at pH 6.0

| | | koff (1/s) for |
|---|---|---|
| ISVD name | Substitutions (Kabat) | human FcRn at pH 6.0 |
| T0263018B11 | Parental | 8.0E−04 |
| T026301049 | G26T, S31E, A33D, S100D | no binding |
| T026301050 | G26T, S52aQ, S100D | 2.9E−02 |
| T026301051 | A33D, S100D | 1.2E−02 |
| T026301052 | G26T, S31E, A33D | 5.6E−03 |
| T026301053 | G26T, S31E, A33D, W100cK | no binding |
| T026301054 | G26T, S31E, S52aQ, S100D | 4.1E−02 |
| T026301055 | G26T, A33D, W100cK | no binding |
| T026301056 | G26T, S31E, A33D, S52aQ, | no binding |
| | S100D, W100cK | |
| T026301057 | S31E, S52aQ, W100cS | 1.6E−02 |
| T026301058 | G26T, S31E, S52aQ, S100D, | no binding |
| | W100cK | |
| T026301059 | S31E, A33D, S52aQ, S100D, | 3.9E−02 |
| | W100cS | |
| T026301060 | S31E, S100D, W100cS | 3.4E−02 |
| T026301061 | G26T, S100D | 1.1E−02 |
| T026301062 | G26T, A33D, S100D | no binding |
| T026301063 | G26T, S52aQ | 5.0E−03 |
| T026301064 | A33D, S52aQ | 4.6E−04 |
| T026301065 | G26T, S31E, A33D, S52aQ, | no binding |
| | S100D | |
| T026301066 | S31E, S100D, W100cK | no binding |
| T026301067 | G26T, A33D, S52aQ, W100cK | 6.1E−03 |
| T026301068 | A33D, S52aQ, S100D, W100cK | no binding |
| T026301069 | A33D, S52aQ, W100cS | 1.7E−03 |
| T026301070 | S31E, A33D, S52aQ, W100cS | 4.6E−03 |
| T026301071 | G26T, S31E, S100D, W100cS | 4.9E−02 |
| T026301072 | A33D, S52aQ, S100D, W100cS | 1.5E−02 |
| T026301073 | S100E | 3.2E−03 |
| T026301074 | G26T, S31E, S100D | 1.3E−02 |
| T026301075 | G26T, S31E | 2.3E−03 |
| T026301076 | A33D, S52aQ, W100cK | 3.0E−03 |
| T026301077 | G26T, S31E, A33D, S52aQ, | no binding |
| | W100cK | |
| T026301078 | S31E, A33D, S52aQ, S100D | 9.5E−03 |
| T026301079 | S52aQ, W100cS | 6.5E−03 |
| T026301080 | A33D, W100cS | 5.1E−03 |
| T026301081 | G26T, W100cS | 8.8E−03 |
| T026301082 | G26T, S100D, W100cS | 5.4E−02 |
| T026301083 | S31E, S52aQ, S100D, W100cS | 6.7E−02 |
| T026301084 | S100D, W100cS | 2.1E−02 |
| T026301085 | S52aQ, S100D | 6.6E−03 |
| T026301086 | S31E, A33D, S100D | 1.3E−02 |
| T026301087 | S31E, A33D, S52aQ | 1.1E−03 |
| T026301088 | A33D, W100cK | 9.3E−03 |
| T026301089 | S100D, W100cK | 2.8E−02 |
| T026301090 | G26T, S52aQ, W100cK | 3.3E−02 |
| T026301091 | S31E, A33D, W100cS | 6.2E−03 |
| T026301092 | A33D, S100D, W100cK | no binding |
| T026301093 | G26T, A33D, W100cS | no binding |
| T026301094 | G26T, S31E, A33D, S52aQ, | 1.0E−02 |
| | W100cS | |
| T026301095 | S31E, A33D, S100D, W100cS | no binding |
| T026301096 | S52aQ, S100D, W100cS | 3.4E−02 |
| T026301097 | G26T, S31E, S52aQ | 6.7E−03 |
| T026301098 | S31D | 1.3E−02 |
| T026301099 | S31E, A33D | 1.5E−03 |
| T026301100 | A33D, S52aQ, S100D | 3.9E−03 |
| T026301101 | G26T, S100D, W100cK | no binding |
| T026301102 | S31E, A33D, W100cK | 1.1E−02 |
| T026301103 | G26T, S52aQ, W100cS | 2.1E−02 |
| T026301104 | S31E, S52aQ, S100D, W100cK | no binding |
| T026301105 | G26T, S31E, W100cS | 1.1E−02 |
| T026301106 | G26T, A33D, S52aQ, S100D, | no binding |
| | W100cS | |
| T026301107 | G26T, A33D, S52aQ, W100cS | 4.5E−03 |
| T026301108 | G26T, S31E, S52aQ, S100D, | no binding |
| | W100cS | |
| T026301109 | G26T, A33D, S52aQ | 1.3E−03 |
| T026301110 | S31E, S52aQ, S100D | 1.4E−02 |
| T026301111 | S31E, S52aQ | 3.2E−03 |
| T026301112 | S31E, A33D, S52aQ, W100cK | 7.2E−03 |
| T026301113 | S31E, S52aQ, W100cK | 2.1E−02 |
| T026301114 | G26T, S31E, S100D, W100cK | no binding |
| T026301115 | G26T, A33D, S100D, W100cK | no binding |
| T026301116 | S31E, A33D, S100D, W100cK | no binding |
| T026301117 | S31E, W100cS | 6.7E−03 |
| T026301118 | A33D, S100D, W100cS | no binding |
| T026301119 | G26T, S52aQ, S100D, W100cS | 1.1E−01 |
| T026301120 | G26T, S31E, A33D, S52aQ, | no binding |
| | S100D, W100cS | |
| T026301121 | G26T, S31E, A33D, S52aQ | 1.8E−03 |
| T026301122 | W100cR | 5.7E−03 |
| T026301123 | G26T, A33D | 5.4E−03 |
| T026301124 | G26T, S31E, S52aQ, W100cK | 5.9E−02 |
| T026301125 | S31E, W100cK | 8.2E−03 |
| T026301126 | G26T, W100cK | 1.0E−02 |
| T026301127 | G26T, S31E, A33D, W100cS | 9.5E−03 |
| T026301128 | S52aQ, S100D, W100cK | 3.2E−02 |
| T026301129 | S31E, A33D, S52aQ, S100D, | no binding |
| | W100cK | |
| T026301130 | G26T, S31E, A33D, S100D, | no binding |
| | W100cS | |
| T026301131 | G26T, A33D, S100D, W100cS | no binding |

TABLE 6
Affinity of T0263018B11 combinatorial variants for human and cyno FcRn at pH 6.0 and pH 7.4

| | | | | | | | | Selected |
|---|---|---|---|---|---|---|---|---|
| | | human FcRn | | | Cyno FcRn | | | for |
| | | | | pH | | | pH | formatting |
| | Substitutions | pH 6.0 | pH 7.4 | 7.4/6.0 | pH 6.0 | pH 7.4 | 7.4/6.0 | and in vivo |
| ISVD name | (Kabat) | KD (M) | KD (M) | Ratio | KD (M) | KD (M) | Ratio | testing |
| T026301057 | S31E, S52aQ, | 3.9E−07 | | NA | 2.6E−07 | | NA | |
| | W100cS | | | | | | | |
| T026301063 | G26T, S52aQ | 6.2E−08 | | NA | 4.1E−08 | | NA | |
| T026301064 | A33D, S52aQ | 9.4E−09 | 3.7E−06 | 394 | 3.8E−09 | 2.4E−06 | 632 | |
| T026301069 | A33D, S52aQ, | 5.5E−08 | | NA | 2.3E−08 | | NA | |
| | W100cS | | | | | | | |
| T026301070 | S31E, A33D, | 1.5E−07 | | NA | 1.4E−07 | | NA | |
| | S52aQ, W100cS | | | | | | | |
| T026301073 | S100E | 1.2E−07 | | NA | 5.9E−08 | | NA | Yes |
| T026301075 | G26T, S31E | 4.7E−08 | 5.6E−06 | 119 | 2.1E−08 | 3.1E−06 | 148 | |
| T026301076 | A33D, S52aQ, | 1.2E−07 | | NA | 3.7E−08 | | NA | |
| | W100cK | | | | | | | |
| T026301079 | S52aQ, W100cS | 1.2E−07 | | NA | 7.6E−08 | 5.9E−06 | 78 | |
| T026301080 | A33D, W100cS | 1.5E−07 | | NA | 1.3E−07 | | NA | |
| T026301087 | S31E, A33D, | 2.5E−08 | | NA | 2.0E−08 | | NA | |
| | S52aQ | | | | | | | |
| T026301091 | S31E, A33D, | 2.5E−07 | | NA | 1.6E−07 | | NA | Yes |
| | W100cS | | | | | | | |
| T026301098 | S31D | 2.0E−07 | | NA | 1.4E−07 | | NA | |
| T026301099 | S31E, A33D | 3.0E−08 | | NA | 3.0E−08 | | NA | |
| T026301100 | A33D, S52aQ, | 1.4E−07 | | NA | 9.8E−08 | | NA | |
| | S100D | | | | | | | |
| T026301103 | G26T, S52aQ, | 2.9E−07 | | NA | 1.3E−07 | | NA | Yes |
| | W100cS | | | | | | | |
| T026301111 | S31E, S52aQ | 7.7E−08 | 5.4E−06 | 70 | 4.8E−08 | 5.0E−06 | 104 | |
| T026301117 | S31E, W100cS | 1.7E−07 | | NA | 1.1E−07 | | NA | |
| T026301122 | W100cR | 1.6E−07 | | NA | 8.1E−08 | | NA | |
| T0263018B11 | Parental | 7.3E−09 | 6.0E−07 | 82 | 7.2E−09 | 4.3E−07 | 60 | yes |

Empty cell: no affinity determination possible due to low binding; italics and underlined: indicative values;
NA: Not applicable.

### 6.5 Example 5: Affinity Determination Via Surface Plasmon Resonance (SPR) of Pentavalent ISVDs

Certain of the obtained mutant variants of the T0263018B11 ISVD building block were selected for further formatting by linkage with one or more reference ISVD building blocks (“REF”, not binding to FcRn) to generate pentavalent ISVD formats. The affinity of the purified, pentavalent ISVD constructs for human and cyno FcRn was determined by a similar assay and under the same conditions as described for the monovalent ISVDs in Example 3. The affinities are shown in Table 7.

TABLE 7
Affinity of pentavalent ISVD formats for human and cyno FcRn at pH 6.0 and pH 7.4

| Pentavalent | | | K_{D} (M) on | | K_{D} (M) on | |
|---|---|---|---|---|---|---|
| ISVD | FcRn binding ISVD building | | human FcRn | | cyno FcRn | |
| construct | block (FcRn) present in | | pH | pH | pH | pH |
| name | construct | Format | 6.0 | 7.4 | 6.0 | 7.4 |
| T026301220 | T0263018811(E1D, G26T, S52aQ, | (T0263018B11(E1D, G26T, S52aQ, | 3.7E−08 | 9.9E−07 | 2.6E−08 | 6.7E−07 |
| | W100cS) | W100cS))₂-(REF)₃ | | | | |
| T026301230 | T0263018B11(E1D, W100cS) | T0263018B11(E1D, W100cS)- | 1.0E−07 | 6.7E−07 | 5.4E−08 | 5.8E−07 |
| | | (REF)₄ | | | | |
| T026301233 | T0263018B11(E1D, G26T, S52aQ, | T0263018B11(E1D, G26T, S52aQ, | 4.9E−07 | 7.5E−07 | 2.7E−07 | 4.2E−07 |
| | W100cS) | W100cS)-(REF)₄ | | | | |
| T026301234 | T0263018B11(E1D) | T0263018B11(E1D) (REF)₄ | 2.6E−08 | 7.4E−07 | 1.4E−08 | 4.9E−07 |

Italics and underlined: indicative data

### 6.6 Example 6: Structural Interaction of the FcRn Binding ISVD T0263018B11 with FcRn

The structure of the interaction of the FcRn binding ISVD T0263018B11 with human FcRn(24-297; N125Q)/b2M(21-119) was determined via co-crystallization of the complex. The human FcRn(24-297; N125Q)/b2M(21-119) protein was produced in mammalian cells and contains the N125Q mutation to avoid N-glycosylation of the protein.

The complex was concentrated to 9.5 mg/ml in 10 mM Histidine, 150 mM NaCl pH 6. Crystallization was done using the sitting drop method. 18% PEG80000, 200 mM NaCl, 50 mM TRIS pH 8.0 at 19° C., 25% ethylene glycol was included as cryoprotectant prior freezing. Datasets were collected from both crystals at beamline Proximal from the synchrotron SOLEIL on a EIGRR-X 6M detector (Dectris Ltd.). Crystals belong to the same space group C2 and diffracted to 1.64 Å. Data was processed using autoproc (Vonrhein, C., Flensburg, C., Keller, P., Sharff, A., Smart, O., Paciorek, W., Womack, T. & Bricogne, G. (2011)). Data processing and analysis with the autoPROC toolbox. Acta Cryst. D67, 293-302.) which relies on XDS (Kabsch, W. XDS. Acta Cryst. D66, 125-132 (2010)) and Aimless (P. R. Evans and G. N. Murshudov, ‘How good are my data and what is the resolution?’ Acta Cryst. D69, 1204-1214 (2013)). A model of FcRn (24-297; N125Q)/b2M(21-119) was obtained using the pdb structure 4NOU as reference. A model of FcRn binding ISVD T0263018B11 was constructed in Maestro (Schrodinger Release 2021-4: Maestro, Schrodinger, LLC, New York, NY, 2021). Molecular replacement was carried out using Phaser (Coy et al, J. Appl. Cryst. (2007) 40, 658-674) of the CCP4 suite (Winn et al, Acta Cryst D67 (2011), 235-242). The structure was refined at 1.64A using cycles of Buster (Buster-TNT 2.11.5, Global Phasing Ltd) followed by manual corrections in COOT (Emsley, P. & Cowtan, K. (2004). Acta Cryst. D60, 2126-2132.) to a final Rfree of 24.3% and Rfactor 20.7%.

Rsym and Rmerge—the formula for both is:

$$R = \frac{{Σ}_{hkl} {Σ}_{j} \vert {I}_{hkl . , j} - 〈 {I}_{hkl} 〉 \vert}{{Σ}_{hkl} {Σ}_{j} {I}_{hkl , j}}$$

where is the average of symmetry- (or Friedel-) related observations of a unique reflection.
R and Rfree:

$$R = \frac{{Σ}_{hkl} \vert {F}_{hkl}^{obs} - {F}_{hkl}^{calc} \vert}{{Σ}_{hkl} {F}_{hkl}^{obs}}$$

where Fₕₖₗ^{obs} and Fₕₖₗ^{calc} have to be scaled w.r.t. each other. R and Rfree differ in the set of reflections they are calculated from (https://strucbio.biologie.uni-konstanz.de/ccp4wiki/index.php?title=R-factors).

The residues involved in the epitope-paratope were determined with the suite of software Schrodinger Release 2021-4: Maestro, Schrödinger, LLC, New York, NY, 2021. The FcRn epitope bound by T0263018B11 was compared with the epitopes bound by the natural ligands HSA and IgG as present in PDB (Protein Database) ID: 4NOU and the molecular surface representations are shown in FIGS. 2A-2D and 3A-3D (PyMOL 2.3.0).

### 6.7 Example 7: Generation of Multivalent ISVD Constructs Containing Serum Albumin Binding ISVD ALB23002 and Affinity Determination Via Surface Plasmon Resonance (SPR)

Based on the FcRn affinities at pH 6.0 and pH 7.4, multiple ISVDs were selected for formatting with the HSA binding ISVD building block ALB23002 (SEQ ID NO.: 35). The FcRn binding ISVD building block was linked to ALB23002 with a 35GS linker (SEQ ID NO.: 48) and a C-terminal alanine was added. In certain formats, additional target ISVD building blocks (X,Y) were also added to represent possible future therapeutic leads. These formats were expressed in P. pastoris and purified as described in Examples 3.1 and 3.2.

The affinity of the purified, multivalent ISVD constructs for human and cyno FcRn was determined on the MASS-2 (Sierra sensors, Gmbh) instrument, hFcRn/hb2M and cFcRn/cb2M was captured on a Biotin-tag Capture Chip or a High Capacity Amine Chip (pre-immobilized with neutravidin). The ISVD constructs were injected at 6 different concentrations (between 1 and 4000 nM) and allowed to associate for 120 s at 45 μL/min and dissociate for 600 s-3600 s at 45 μL/min. Higher ISVD construct concentrations were used to determine the affinity at pH 7.4 compared to pH 6.0, due to the lower binding at pH 7.4. Evaluation of the sensorgrams was based on the 1:1 Langmuir dissociation model simultaneously fitting on and off-rates. The affinities are shown in Table 8 and Table 9.

TABLE 8
Affinity of constructs comprising an FcRn binding ISVD (FcRn) and serum albumin
binding ISVD (ALB23002) for human and cyno FcRn at pH 6.0 and pH 7.4

Name of ISVD

| construct (FcRn- | FcRn binding ISVD building | K_{D} (M) on human FcRn | | K_{D} (M) on cyno FcRn | |
|---|---|---|---|---|---|
| 35GS-ALB23002-A) | block used in construct | pH 6.0 | pH 7.4 | pH 6.0 | pH 7.4 |
| T026301161 | T0263046C05(E1D) | 6.8E−09 | NA | 6.7E−09 | NA |
| T026301163 | T0263089E01(E1D) | 2.1E−07 | NA | 1.7E−07 | NA |
| T026301168 | T0263091G06(E1D) | 1.8E−06 | NA | 8.1E−07 | NA |
| T026301170 | TO263018811(E1D) | 1.2E−08 | 4.5E−07 | 7.9E−09 | 2.9E−07 |
| T026301185 | T0263066B10(E1D) | 2.5E−07 | 6.5E−06 | 5.7E−08 | 8.4E−07 |
| T026301192 | T0263018811(E1D, | 2.5E−07 | very low | 1.4E−07 | 4.8E−07 |
| | S100E) | | binding | | |
| T026301195 | T0263018B11(E1D, | 6.8E−07 | no binding | 6.7E−07 | no |
| | S31E, A33D, W100cS) | | | | binding |
| T026301196 | T0263018811(E1D, | 1.0E−06 | very low | 5.1E−07 | very low |
| | G26T, S52aQ, W100cS) | | binding | | binding |
| T026301197 | T0263018811(E1D, S52aQ) | 7.1E−08 | 2.6E−06 | 4.1E−08 | 2.4E−06 |
| T026301198 | T0263018B11(E1D, W100cS) | 1.1E−07 | 2.5E−06 | 6.7E−08 | 2.9E−06 |

NA: not available;
italics and underlined: indicative values

TABLE 9
Affinity of constructs comprising an FcRn binding ISVD (FcRn), a serum albumin binding ISVD (ALB23002),
and additional target binding ISVD building blocks (X, Y) for human and cyno FcRn at pH 6.0 and pH 7.4

| | FcRn binding | | | | | |
|---|---|---|---|---|---|---|
| | ISVD building | | | | | |
| Name of ISVD | block used | | K_{D} (M) on human FcRn | | K_{D} (M) on cyno FcRn | |
| construct | in construct | Format | pH 6.0 | pH 7.4 | pH 6.0 | pH 7.4 |
| T026301257 | T0263018B11 | X-Y-FcRn-35GS- | 3.4E−08 | 1.2E−06 | 2.0E−08 | 7.1E−07 |
| | | ALB23002 | | | | |
| T026301259 | T0263018B11 | X-Y-FcRn-ALB23002 | 2.5E−08 | 8.3E−07 | 1.5E−08 | 4.8E−07 |
| T026301266 | T0263018B11 | X-FcRn-Y-ALB23002 | 4.1E−08 | 1.6E−06 | 2.6E−08 | 8.8E−07 |
| T026301366 | T0263018B11(W100cS) | X-Y-FcRn-35GS- | 3.1E−07 | NA | 1.8E−07 | NA |
| | | ALB23002 | | | | |

NA: not available

The affinity of the purified, multivalent ISVD constructs for human and mouse serum albumin (HSA and MSA, respectively) was determined on the ProteOn XPR36 (BioRad Laboratories, Inc.) instrument. HSA/MSA (HSA: Sigma-Aldrich—Sigma, Cat No. A8763; MSA: Albumin Bioscience, Cat No. 2601) was immobilized on a ProteOn GLC sensor chip (Biorad Laboratories, 1765011). The ISVD constructs were injected at 6 different concentrations (between 1 and 500 nM) and allowed to associate for 120 s at 45 μL/min and dissociate for 600s-3600s at 45 μL/min. Evaluation of the sensorgrams was based on the 1:1 Langmuir dissociation model simultaneously fitting on and off-rates. The affinities are shown in Table 10 and Table 11.

TABLE 10
Affinity of constructs comprising an FcRn binding ISVD (FcRn) and a serum albumin
binding ISVD (ALB23002) for human and mouse serum albumin at pH 6.0 and pH 7.4

Name of ISVD

| construct (FcRn- | FcRn binding ISVD building | K_{D} (M) on HSA | | K_{D} (M) on MSA | |
|---|---|---|---|---|---|
| 35GS-ALB23002-A) | block used in construct | pH 6.0 | pH 7.4 | PH 6.0 | PH 7.4 |
| IRR00122 | Reference Nanobody clone | 5.4E−09 | 1.2E−08 | 5.8E−08 | 9.0E−08 |
| T026301161 | T0263046C05(E1D) | 7.1E−09 | 1.5E−08 | 7.4E−08 | 1.3E−07 |
| T026301163 | T0263089E01(E1D) | 6.6E−09 | 1.3E−08 | 9.6E−08 | 1.3E−07 |
| T026301168 | TO263091G06(E1D) | 8.5E−09 | 1.6E−08 | 1.0E−07 | 1.4E−07 |
| T026301170 | T0263018B11(E1D) | 6.1E−09 | 1.3E−08 | 6.7E−08 | 8.5E−08 |
| T026301185 | T0263066B10(E1D) | 5.0E−09 | 1.0E−08 | 6.7E−08 | 9.6E−08 |
| T026301192 | T0263018811(E1D, S100E) | 5.7E−09 | 1.2E−08 | 6.4E−08 | 8.7E−08 |
| T026301195 | T0263018811(E1D, S31E, | 6.1E−09 | 1.3E−08 | 7.7E−08 | 1.2E−07 |
| | A33D, W100cS) | | | | |
| T026301196 | TO263018B11(E1D, G26T, | 7.4E−09 | 1.5E−08 | 8.9E−08 | 1.2E−07 |
| | S52aQ, W100cS) | | | | |
| T026301197 | T0263018811(E1D, S52aQ) | 5.1E−09 | 1.1E−08 | 5.7E−08 | 6.9E−08 |
| T026301198 | T0263018B11(E1D, W100cS) | 7.3E−09 | 1.5E−08 | 8.9E−08 | 1.2E−07 |

italics and underlined: indicative values

TABLE 11
Affinities and off-rates of constructs comprising an FcRn binding ISVD (FcRn),
a serum albumin binding ISVD (ALB23002), and additional target binding ISVD
building blocks (X, Y) for human and mouse serum albumin at pH 6.0 and pH 7.4

| Name of | FcRn binding | | | | | |
|---|---|---|---|---|---|---|
| Nanobody ® | Nanobody ® | | | | | |
| ISVD | ISVD building | | | | | |
| construct | block used in | | | | | |
| (FcRn-35GS- | construct FcRn | | K_{D} (M) on HSA | | k_{d} (1/s) on MSA | |
| ALB23002-A) | Nanobody ® clone | Format | pH 6.0 | pH 7.4 | pH 6.0 | pH 7.4 |
| T026301257 | T0263018B11 | X-Y-FcRn-35GS-ALB | 1.4E−08 | 1.1E−08 | 5.6E−02 | 5.2E−02 |
| T026301259 | T0263018B11 | X-Y-FcRn-ALB | 9.8E−09 | 7.32E−09 | 3.8E−02 | 3.7E−02 |
| T026301266 | T0263018B11 | X-FcRn-Y-ALB | 1.2E−08 | 8.6E−09 | 5.3E−02 | 4.8E−02 |
| T026301366 | T0263018B11(W100cS) | X-Y-FcRn-35GS-ALB | 1.0E−08 | 7.5E−09 | 3.4E−02 | 3.5E−02 |

### 6.8 Example 8: Development and Optimization of Serum PK Assays

Pharmacokinetic experiments were initiated in TG32 (B6.Cg-Fcgrttm1Dcr Tg(FCGRT) 32Dcr/Dcr) mice to evaluate half-life of pH-dependent FcRn binding ISVDs genetically fused to either a serum albumin binding ISVD (ALB23002, SEQ ID NO.: 35) alone or in combination with additional target binding ISVDs (X, Y), or to control ISVDs (REF). A specific and sensitive ligand binding assay was developed to measure concentrations of all constructs in mouse serum.

A streptavidin-coated MSD GOLD 96-well SMALLSPOT® plate (Meso Scale Discovery) was blocked with Superblock T20™ (Thermo Scientific) for 30 minutes at RT. The plate was then washed and incubated for 1 hour at RT and at 600 rpm with 1.0 pg/mL biotinylated generic mAb directed against the frameworks of the different ISVD building blocks. Calibrators and QCs were prepared in pooled mouse serum. After washing the plate, calibrators, QCs and samples were applied to the plate at an MRD of 20 in PBS 0.1% casein and incubated for 1 hour at RT and at 600 rpm. After washing, the plate was incubated for 1 hour at RT and at 600 rpm with 2.0 μg/mL sulfo-labelled mAb directed against a specific ISVD building block, depending on the format under evaluation. After the plate was washed, 2×MSD Read buffer (Meso Scale Discovery) was added and the plate was read on a Sector Imager Quickplex SQ 120 (Meso scale Discovery).

For in vivo experiments in which a mixture of human IgGs (hIVIG; Privigen®) was used to mimic endogenous IgG competition, hIgG assay interference was evaluated.

### 6.9 Example 9: Pharmacokinetics of Bispecific ISVD Constructs in Transgenic Mice

Six to eight Tg32 mice (B6.Cg-Fcgrttm1Dcr Tg(FCGRT) 32Dcr/DcrJ) were injected intravenously in the tail with 1.9 mg/kg bispecific ISVD constructs. Bispecific ISVD constructs consisting of 2 genetically fused ISVD domains, the N-terminal one being either a negative control (IRR) ISVD or an FcRn-targeting ISVD (FcRn) and the C-terminal being an albumin-targeting ISVD (Alb23002).

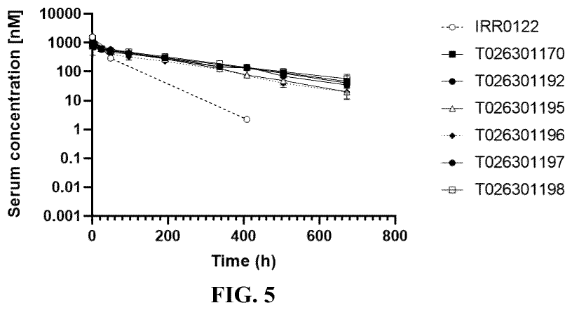

Blood was retrieved at different time points (2 mice per time point) and serum was prepared. Serum samples were analyzed by ELISA for the presence of bispecific ISVD construct as described in Example 8. Results are shown in FIG. 4.

Half-lives values were obtained by estimating the in vivo endosomal FcRn affinities in a mechanistic model and reported in Table 12.

We can conclude from the results that the half-life of bispecific ISVD comprising an FcRn-binding and an albumin binding ISVD can be significantly improved compared to constructs comprising an albumin binding ISVD only. Half-lives of up to 8.2 days (for T026301170, containing FcRn binding ISVD T0263018B11) have been observed for FcRn-Alb23002 bi-specific ISVDs compared to 2.4 days for the control (IRR-Alb23002).

TABLE 12
Calculated half-lives (days) of bispecific ISVD constructs (anti-FcRn-Alb23002 format)

| | IRR00122 | T026301170 | T026301185 | T026301168 | T026301163 | T026301161 |
|---|---|---|---|---|---|---|
| T_{1/2} (days) | 2.4 | 8.2 | 5.3 | 2.7 | 2.1 | 1.2 |

### 6.10 Example 10: Pharmacokinetics in Mice of Bispecific ISVD Constructs Comprising Mutant Variants of T0263018B11

Two to eight Tg32 mice (B6.Cg-Fcgrttm1Dcr Tg(FCGRT) 32Dcr/DcrJ) were injected intravenously in the tail with 1.9 mg/kg bispecific ISVD constructs. Bispecific ISVD constructs consisting of 2 genetically fused ISVD domains, the N-terminal one being either a negative control (IRR) ISVD or an FcRn-targeting ISVD (FcRn) and the C-terminal being an albumin-targeting ISVD (ALB23002). The FcRn-targeting ISVD was either a parental sequence, or a mutant variant with reduced affinity at neutral pH (see Example 4).

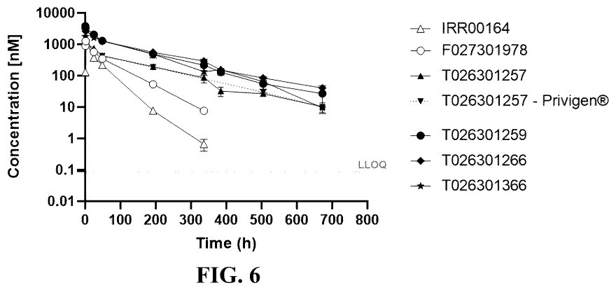

Blood was retrieved at different time points (2 mice per time point) and serum was prepared. Serum samples were analyzed by ELISA for the presence of bispecific ISVD construct as described in Example 8. Results are shown in FIG. 5. Half-lives values were obtained by estimating the in vivo endosomal FcRn affinities in a mechanistic PBPK model and reported in Table 13.

We can conclude from the results that in bispecific ISVD constructs comprising mutant variants of T0263018B11 and an albumin binding ISVD, half-life is significantly improved compared to the control (IRR-Alb23002-IRR0122) ISVD.

For all evaluated mutant variants, half-life was improved at least by 2.2-fold and at most by 3.7-fold compared to the control (IRR-Alb).

TABLE 13
Calculated half-lives of bispecific ISVD constructs (anti-FcRn T0263018B11-
Alb23002 format or mutant variant of T0263018B11-Alb23002 format)

| | IRR00122 | T026301170 | T026301192 | T026301195 | T026301196 | T026301197 | T026301198 |
|---|---|---|---|---|---|---|---|
| T_{1/2} | 2.4 | 8.2 | 7.5 | 5.4 | 6.2 | 7.7 | 8.8 |
| (days) | | | | | | | |

### 6.11 Example 11: Pharmacokinetics in Mice of Multivalent ISVD Constructs Comprising an FcRn Binding ISVD (FcRn), a Serum Albumin Binding ISVD (ALB23002), and Additional Target Binding ISVD Building Blocks (X,Y)

To mimic relevant competition with hIgG, Tg32 mice (B6.Cg-Fcgrttm1Dcr Tg(FCGRT) 32Dcr/DcrJ) were preloaded with a mixture of purified hIgG (hIVIG; Privigen®). Privigen® was administered intravenously once weekly, with the first administration 2 days prior to initiation of the PK study. In total, 3 Privigen® injections of 250 mg/kg were administered, yielding physiologically relevant hIgG serum concentrations for the duration of the study (data not shown). All groups received Privigen® treatment, and one additional group was included where Privigen® was not administered. This group allowed evaluation of the impact of hIgG on PK, which could be expected in case of competition for the same epitope of the FcRn binding ISVD and IgG Fc.

Two days after the first Privigen® administration, 4 to 6 Tg32 mice (B6.Cg-Fcgrttm1Dcr Tg(FCGRT) 32Dcr/DcrJ) were injected intravenously in the tail with equimolar amounts of multivalent ISVD constructs consisting of 3 to 4 genetically fused ISVD domains (formats and building blocks used in these constructs were described in Example 7 above). Each construct contained 2 ISVD domains directed against different potential therapeutically relevant targets (X,Y) fused to either an albumin-binding ISVD (Alb23002) alone (trivalent) or a combination of an FcRn-binding ISVD (FcRn) and an albumin-binding ISVD (Alb23002) (tetravalent). In addition, a tetravalent control ISVD containing 3 non-targeting ISVDs (IRR) and one albumin-binding ISVD (Alb23002) was administered (IRR00164).

Blood was retrieved at different time points (2 mice per time point) and serum was prepared. Serum samples were analyzed by ELISA for the presence of multivalent ISVD constructs as described in Example 7.

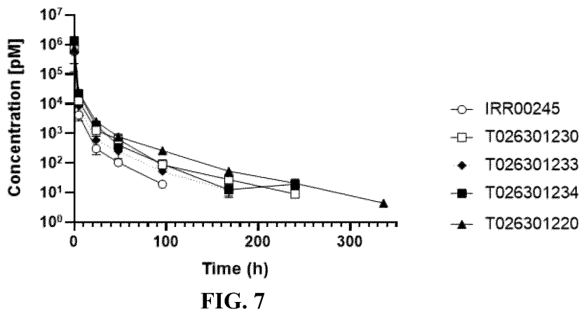

Results are shown in FIG. 6. Half-lives values were obtained by estimating the in vivo endosomal FcRn affinities in a mechanistic PBPK model and reported in Table 14.

In multivalent ISVD constructs comprising ISVD domains against potentially relevant therapeutic targets, the combination of an FcRn binding ISVD with an albumin binding ISVD significantly improved half-life compared to the control ISVD constructs containing only an albumin binding ISVD. Position of the FcRn binding ISVD, as well as linker length between the FcRn binding ISVD and the albumin binding ISVD did not impact the pharmacokinetic properties (half-life) of the constructs. In addition, half-life was not impacted by the presence of relevant levels of hIgG, suggesting that a different epitope is bound by the FcRn binding ISVD compared to the epitope bound by an IgG Fc. For all evaluated multivalent ISVD constructs, half-life was improved with 2-fold compared to the control ISVD formats.

TABLE 14
Formats and calculated half-lives of multivalent ISVD constructs evaluated in Tg32 mice

| | IRR00164 | F027301978 | T02601257* | T026301259 | T026301266 | T026301366^{$} |
|---|---|---|---|---|---|---|
| Format | IRR-IRR-IRR- | X-Y- | X-Y-FcRn- | X-Y-FcRn- | X-FcRn-Y- | X-Y-FcRn- |
| | Alb23002 | Alb23002 | Alb23002 | Alb23002 | Alb23002 | Alb23002 |
| Linker | NA | NA | 35GS | 9GS | NA | 35GS |
| between | | | | | | |
| FcRn-Alb | | | | | | |
| T_{1/2} | 1.5 | 2.4 | 4.9 | 4.4 | 5.3 | 4.1 |
| (days) | | | | | | |

*T02601257 was evaluated both in presence and absence of Privigen ®
^{$}T026301366 contained a pH-engineered variant of the FcRn-binding VHH 18B11

### 6.12 Example 12: Pharmacokinetics of Multivalent FcRn Binding ISVD Constructs Comprising pH-Engineered Variants of T0263018B11

Six to nine Tg32 mice (B6.Cg-Fcgrttm1Dcr Tg(FCGRT) 32Dcr/DcrJ) were injected intravenously in the tail with 4.5 mg/kg multivalent ISVD constructs. Multivalent ISVD constructs consisted of 5 genetically fused ISVD domains, comprising either one or two FcRn-targeting ISVDs (FcRn) and negative control (IRR) ISVD or a control construct comprising only negative control (IRR) ISVD (IRR00245). The FcRn-targeting ISVD was either a parental sequence, or a mutant variant with reduced affinity at physiologic pH (see Example 4).

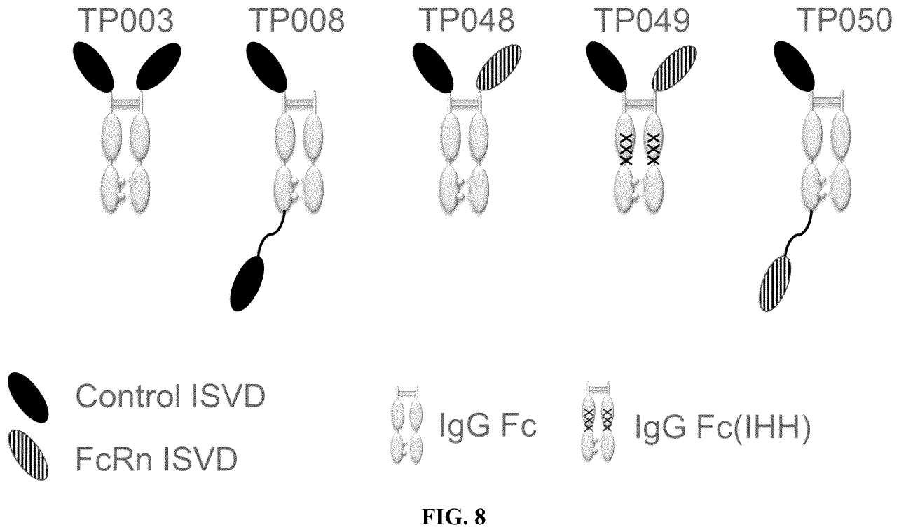

Blood was retrieved at different time points (2-3 mice per time point) and serum was prepared. Serum samples were analyzed by ELISA for the presence of ISVD construct as described in Example 8. Results are shown in FIG. 7. Half-lives values were obtained by estimating the in vivo endosomal FcRn affinities in a mechanistic PBPK model and reported in Table 15.

Multivalent ISVD constructs comprising T0263018B11 or mutant variants of T0263018B11 showed improved half-life compared to the control (IRR00245) ISVD constructs.

TABLE 15
Formats and calculated half-lives of multivalent
ISVD constructs evaluated in Tg32 mice

| | IRR00245 | T02601230 | T02601233 | T026301234 | T026301220 |
|---|---|---|---|---|---|
| Format | (IRR)₅ | FcRn-(IRR)₄ | FcRn-(IRR)₄ | FcRn-(IRR)₄ | (FcRn)₂-(IRR)₃ |
| T_{1/2} (hours) | 15 | 48 | 41 | 102* | 49 |

*higher degree of uncertainty due to poor curve fit

### 6.13 Example 13: Composition of the Polypeptide Constructs of the Invention

### 6.13.1 Fc Domains in the Polypeptides According to Particular Embodiments of the Present Invention

The polypeptides according to the present invention (multi-specific polypeptides, as described above) may further comprise an Fc domain of an IgG, as described herein. Fc domain of an IgG refers to the C-terminal non-antigen binding region of an immunoglobulin G heavy chain that contains at least a portion of the constant region. In particular embodiments, the Fc domain can be a native Fc region, i.e., as it occurs in natural antibodies, or it can be a variant Fc region, comprising one or more alterations, mutations or variations as compared to the native Fc domain. In particular embodiments, an Fc domain of an IgG can also be a fragment of a native Fc domain or a fragment of a variant Fc domain.

### 6.13.1.1. Native (i.e., Wild-Type) Fc Domain of Immunoglobulin G (IgG)

In certain embodiments, the polypeptide as described herein comprises a native Fc domain of a human IgG, such as preferably a native Fc of human IgG4 (e.g., Uniprot sequence P01861, SEQ ID NO.: 129). Polypeptides comprising at least one such native Fc domain are produced and tested for beneficial PK properties.

### 6.13.1.2. Variant Fc Domain with Reduced Effector Function

In certain specific embodiments, the polypeptides according to the present invention comprise variant Fc domains which have altered binding properties for an Fc ligand relative to an unmodified parent Fc molecule. For example, a polypeptide described herein may comprise an Fc region having one or more of amino acid residues 234, 235, 236, 237, 297, 318, 320 and 322 substituted to a different amino acid residue, such that the variant Fc region has an altered affinity for an effector ligand, e.g., an Fc receptor or the C1 component of complement, as described in U.S. Pat. Nos. 5,624,821 and 5,648,260, both to Winter et al.

In particular embodiments, the polypeptides of the present invention comprise an Fc variant domain with reduced effector function, in particular the so-called “FALA” or “LALA” Fc mutant with substitution of residues 234 and 235 to alanine. Extra optional mutations include the substitution of arginine residue 409 to lysine, deletion of lysine residue 447. In other embodiments, the Fc variant domain comprises the so-called “FALA” mutations as described herein and the mutation S228P. These Fc variants are referred to as “pFALA”.

Polypeptides comprising at least one Fc domain with the above mutations were produced and tested for beneficial PK properties as described in Example 16 below.

### 6.13.1.3. Variant Fc Domains of IgG with Improved Binding Affinity for the FcRn Receptor

In particular embodiments, the polypeptides according to the present invention comprise an Fc variant domain showing improved binding to the FcRn receptor compared to the native Fc domain. Such Fc variants include those with substitutions at one or more of Fc region residues 259, 308, 428, and 434. Other variants that increase Fc binding to FcRn include: 250E, 250Q, 428L, 428F, 250Q/428L (Hinton et al., 2004, J. Biol. Chem. 279(8): 6213-6216, Hinton et al., 2006 Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276(9):6591-6604).

In certain particular embodiments, the polypeptides according to the present invention comprise an Fc variant domain wherein methionine 428 was substituted to lysine and asparagine 434 was substituted to serine.

Polypeptides comprising at least one Fc domain with the above mutations are produced and tested for beneficial PK properties.

### 6.13.1.4. Variant Fc Domains of IgG with Reduced or No Binding to the FcRn Receptor

In particular embodiments, the polypeptides according to the present invention comprise an Fc variant domain showing reduced or no binding to the FcRn receptor compared to the native Fc domain. Such Fc variants include those with substitutions at one or more of Fc region residues 253, 310 and 453.

In particular embodiments, the polypeptides according to the present invention comprise an Fc variant domain wherein isoleucine 428 was substituted to alanine, histidine 310 was substituted to alanine, and histidine 453 was substituted to alanine, optionally in combination with histidine 453 substituted to alanine.

Polypeptides comprising at least one Fc domain are produced and tested for beneficial PK properties.

### 6.14 Example 14: Generation and Expression of Fusion Protein Constructs Comprising an FcRn Binding ISVD and an IgG4 Fc Domain

Asymmetrical fusion proteins of an FcRn binding Nanobody® VHH (ISVD) linked to an Fc domain of an IgG4 were generated using the Knob-in-Hole technology as commonly known in the art (and as described for instance in patent publication WO 1996/27011 by Genentech as well as scientific publications by Ridgway, J B et al., “‘Knobs-into-holes’ engineering of antibody CH3 domains for heavy chain heterodimerization.” Protein engineering 9, 7 (1996): 617-21 and Merchant et al., “An efficient route to human bispecific IgG” Nature Biotechnology 16, (1998): 677-681).

DNA fragments of a FcRn binding Nanobody® VHH and/or control Nanobody® VHH and an Fc domain of an IgG4, obtained by PCR with specific combinations of forward and reverse primers each carrying a specific Bpil restriction site, were cloned in the appropriate expression vector via Golden Gate cloning (Engler C, Marillonnet S. Golden Gate cloning. Methods Mol Biol. 2014; 1116:119-31). After Sanger sequence confirmation, the plasmid DNA was then transfected into CHOEBNALT85 cells (QMCF Technology) for protein production. The Nanobody® VHH-Fc fusion proteins were purified from the cell supernatants using a protein A capture step followed by an ion exchange and/or size exclusion chromatography purification step.

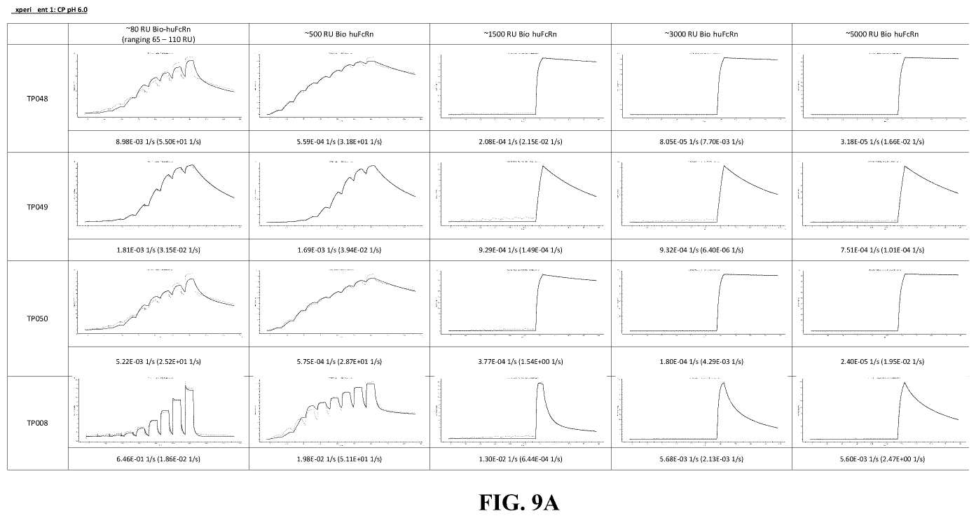

The Fc domains in the constructs were IgG4 pFALA Fc backbone sequence variants (Fc variants with the so-called FALA mutations and the mutation S228P) with knob in hole mutations as described herein whereas the FcRn Nanobody® VHH used was in each case the T0263018B11 sequence as described herein. The Nanobody® VHH sequences in these fusion proteins were fused via a linker (as described in detail herein) to the N- and/or C-terminus of the Fc chain, i.e., via an IgG1 hinge (i.e., see SEQ ID NO.: 126 and/or a GS linker, i.e., 35GS, SEQ ID NO.: 48, respectively (see FIG. 8)). One of these constructs (i.e., TP049) comprises additional amino acid differences or variations in the Fc backbone sequence (i.e., I253A, H310A, H435A), indicated as IgG Fc(IHH) on FIG. 8. This Fc sequence variant was made to test a construct that showed no binding to FcRn via its Fc domain and will be referred to further herein as non-binding Fc-variants. As a control, a Nanobody® VHH-Fc fusion protein was generated, comprising the same composition of the test constructs, except that the Nanobody® VHH binding to FcRn was replaced by a Nanobody® VHH not binding to FcRn or any other envisaged target (i.e., variants of the IgG4 pFALA Fc backbone sequence linked to two Nanobody® VHH's not binding to FcRn, see e.g., FIG. 8, construct TP003 and TP008).

### 6.15 Example 15: FcRn Binding Studies of Nanobody® VHH (ISVD)-Fc Fusion Polypeptide Constructs (IgG4 pFALA)

A set of Nanobody® VHH (ISVD)-Fc fusion proteins was generated that typically consisted of an IgG4 Fc domain linked to (i) a Nanobody® VHH (SVD) specifically binding to FcRn and (ii) to a Nanobody® VHH (ISVD) not binding to FcRn or any other envisaged target but solely included in the polypeptide construct so as to create a similar size (i.e., molecular weight) as the corresponding test construct, as described above (TP003, TP008, TP048, TP049 and TP050).

The Nanobody® VHH-Fc proteins were characterized by affinity determination for human FcRn at pH 6.0 and pH 7.4 on the Biacore 8K+ instrument. For affinity measurements, ˜80 RU of biotinylated human FcRn was captured on a Series S Sensor Chip SA. The Nanobody® VHH-Fc fusion proteins were injected at 9 different concentrations (between 0.5 and 1500 nM) and allowed to associate for 120s at 30 μL/min and dissociate for 600 s. Evaluation of the sensorgrams was based on the Bivalent Analyte fit. The affinity for human FcRn at pH 6.0 and pH 7.4 is shown in Table 16 and Table 17. All constructs showed specific binding to FcRn at pH 6.0 and pH 7.4.

TABLE 16
FcRn affinities of Nanobody ®VHH (ISVD)-Fc fusion constructs at pH 6.0

| Single cycle | | | | | | |
|---|---|---|---|---|---|---|
| kinetics 1 | ka1 | kd1 | ka2 | kd2 | KD1 | KD2 |
| Solution | (1/Ms) | (1/s) | (1/RUs) | (1/s) | (M) | (M) |
| TP003 | 1.4E+05 | 2.2E−01 | 8.3E−05 | 4.9E−04 | 1.5E−06 | 5.9E+00 |
| TP048 | 3.0E+04 | 9.0E−03 | 4.0E+01 | 5.5E+01 | 3.0E−07 | 1.4E+00 |
| TP049 | 1.3E+04 | 1.8E−03 | 1.1E−03 | 3.2E−02 | 1.4E−07 | 2.9E+01 |
| TP050 | 2.3E+04 | 5.2E−03 | 2.1E+01 | 2.5E+01 | 2.2E−07 | 1.2E+00 |

TABLE 17
FcRn affinities of Nanobody ®VHH (ISVD)-Fc fusion constructs at pH7.4

| Single cycle | | | | | | |
|---|---|---|---|---|---|---|
| kinetics 1 | ka1 | kd1 | ka2 | kd2 | KD1 | KD2 |
| Solution | (1/Ms) | (1/s) | (1/RUs) | (1/s) | (M) | (M) |
| TP003 | 9.3E+05 | 1.8E+01 | 5.4E−05 | 2.3E−04 | 1.9E−05 | 4.3E+00 |
| TP048 | 1.6E+04 | 5.6E−02 | 1.2E−05 | 7.8E−04 | 3.5E−06 | 6.4E+01 |
| TP049 | 8.6E+03 | 5.0E−02 | 1.1E−06 | 1.2E−06 | 5.8E−06 | 1.1E+00 |
| TP050 | 7.5E+03 | 6.0E−02 | 4.1E−05 | 3.7E−04 | 8.0E−06 | 9.1E+00 |

### Italics and Underlined: Indicative Values

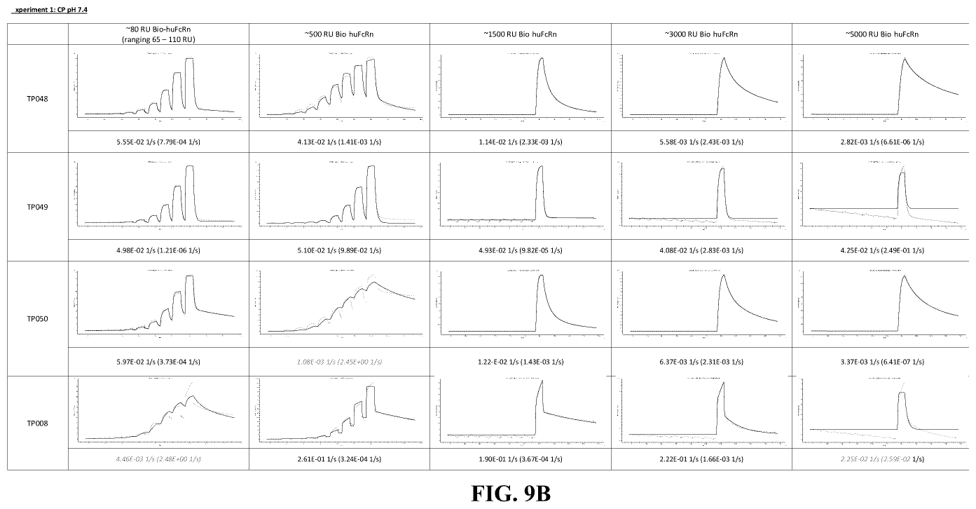

An off-rate analysis was performed at pH 6.0 and pH 7.4 on the Biacore 8K+ instrument using different coating densities of FcRn (˜80, ˜500, ˜1500, ˜3000, ˜5000 RU). For the Fc-fusion constructs that contained the FcRn binding Nanobody® VHH T0263018B11 and an intact Fc (constructs TP048 and TP050), the off-rates (kd1) were slower if the FcRn density on the surfaces was increased (up to 5000 RU, see FIGS. 9A-91B), suggesting an avidity effect through simultaneous FcRn binding by the Nanobody® VHH T0263018B11 and the Fc domain. This slower off-rate was not detected for TP049 containing the non-binding Fc-variant domain (Table 18 and Table 19).

TABLE 18
Off-rates of Nanobody ®VHH-Fc fusion constructs determined on different densities of human FcRn at pH 6.0

| | ~80 RU | | ~500 RU | | ~1500 RU | | ~3000 RU | | ~5000 RU | |
|---|---|---|---|---|---|---|---|---|---|---|
| | kd1 | kd2 | kd1 | kd2 | kd1 | kd2 | kd1 | kd2 | kd1 | kd2 |
| | (s⁻¹) | (s⁻¹) | (s⁻¹) | (s⁻¹) | (s⁻¹) | (s⁻¹) | (s⁻¹) | (s⁻¹) | (s⁻¹) | (s⁻¹) |
| TP048 | 9.0E−03 | 5.5E+01 | 5.6E−04 | 3.2E+01 | 2.1E−04 | 2.2E−02 | 8.1E−05 | 7.7E−03 | 3.2E−05 | 1.7E−02 |
| TP049 | 1.8E−03 | 3.2E−02 | 1.7E−03 | 3.9E−02 | 9.3E−04 | 1.5E−04 | 9.3E−04 | 6.4E−06 | 7.5E−04 | 1.0E−04 |
| TP050 | 5.2E−03 | 2.5E+01 | 5.8E−04 | 2.9E+01 | 3.8E−04 | 1.5E+00 | 1.8E−04 | 4.3E−03 | 2.4E−05 | 2.0E−02 |
| TP008 | 6.5E−01 | 1.9E−02 | 2.0E−02 | 5.1E+01 | 1.3E−02 | 6.4E−04 | 5.7E−03 | 2.1E−03 | 5.6E−03 | 2.5E+00 |

TABLE 19
Off-rates of Nanobody ®VHH-Fc fusion constructs determined on different densities of human FcRn at pH 7.4

| | ~80 RU | | ~500 RU | | ~1500 RU | | ~3000 RU | | ~5000 RU | |
|---|---|---|---|---|---|---|---|---|---|---|
| | kd1 | kd2 | kd1 | kd2 | kd1 | kd2 | kd1 | kd2 | kd1 | kd2 |
| | (s⁻¹) | (s⁻¹) | (s⁻¹) | (s⁻¹) | (s⁻¹) | (s⁻¹) | (s⁻¹) | (s⁻¹) | (s⁻¹) | (s⁻¹) |
| TP048 | 5.6E−02 | 7.8E−04 | 4.1E−02 | 1.4E−03 | 1.1E−02 | 2.3E−03 | 5.6E−03 | 2.4E−03 | 2.8E−03 | 6.6E−06 |
| TP049 | 5.0E−02 | 1.2E−06 | 5.1E−02 | 9.9E−02 | 4.9E−02 | 9.8E−05 | 4.1E−02 | 2.8E−03 | 4.6E−02 | 2.5E−01 |
| TP050 | 6.0E−02 | 3.7E−04 | 1.1E−03 | 2.5E+00 | 1.2E−02 | 1.4E−03 | 6.4E−03 | 2.3E−03 | 3.4E−03 | 6.4E−07 |
| TP008 | 4.5E−03 | 2.5E+00 | 2.6E−01 | 3.2E−04 | 1.9E−01 | 3.7E−04 | 2.2E−01 | 1.7E−03 | 2.3E−02 | 2.6E−02 |

### Italics and Underlined: Indicative Values

### 6.16 Example 16: Development and Optimization of Serum PK Assays

### 6.16.1 Example 16.1. Assay for Detection of ISVD-IgG4 Fc Fusion Polypeptides in Mouse Serum

A specific and sensitive ligand-binding assay was developed in house to detect Nanobody® VHH (ISVD)-IgG4 Fc fusion polypeptides. A streptavidin-coated MSD GOLD 96-well SMALLSPOT® plate (Meso Scale Discovery) was blocked with Superblock T20™ (Thermo Scientific) for 30 minutes at RT. The plate was then washed and incubated for 1 hour at RT and at 600 rpm with 2.0 μg/mL biotinylated generic mAb directed against the frameworks of the ISVD moiety used in each construct. Calibrators and QCs were prepared in pooled mouse serum. After washing the plate. calibrators. QCs and samples were applied at an MRD of 20 to 100 (depending on the construct) in PBS 0.1% casein and incubated for 1 hour at RT and at 600 rpm. After washing. the plate was incubated for 1 hour at RT and at 600 rpm with 2.0 μg/mL sulfo-labelled mAB directed against a specific ISVD moiety. depending on the format under evaluation. After the plate is washed. 2×MSD Read buffer (Meso Scale Discovery) is added and the plate is read on a Sector Imager Quickplex SQ 120 (Meso Scale Discovery). For in vivo experiments in which a mixture of human IgGs (hIVIG; Privigen®) was used to mimic endogenous IgG competition. hIgG assay interference was evaluated.

### 6.16.2 Example 16.2. Pharmacokinetics in Mice with ISVD—IgG4 Fc Fusions

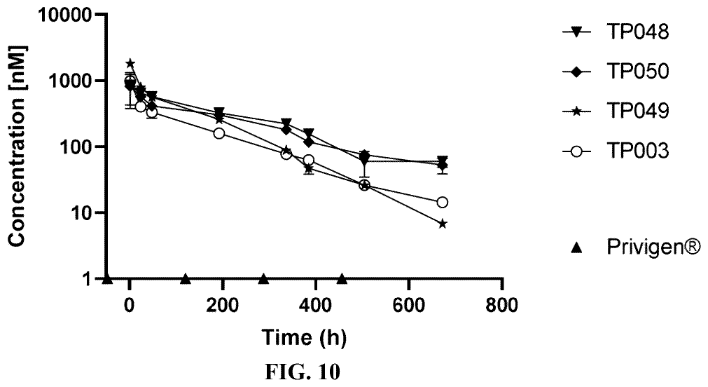

4 to 6 Tg32 mice (B6.Cg-Fcgrttm1Dcr Tg(FCGRT) 32Dcr/DcrJ) were injected intravenously in the tail with 5 mg/kg ISVD-Fc fusions. ISVD-Fc constructs consisted of identical IgG Fc, except for TP049 which had mutations I253A, H310A, H435A (IHH) to abrogate FcRn binding. Fc constructs were genetically fused to 2 ISVD domains. either 2 non-targeting ISVD (negative control; TP003) or 1 non-targeting ISVD and 1 FcRn-targeting ISVD (TP048, TP049 and TP050, see FIG. 8 and Example 14). Blood was retrieved at different time points (2 mice per time point) and serum was prepared. Serum samples were analyzed by ligand binding assay for the presence of Nanobody® VHH-Fc fusions as described in Example 15. Results are shown in FIG. 10.

PK parameters were obtained from non-compartmental analysis in Phoenix WinNonlin® (version 8.2.2.227. Certara) using the Plasma Data Module. Half-live values are reported in Table 20.

We can conclude from the results that N- or C-terminal fusion of an FcRn-binding ISVD to an IgG Fc domain significantly extends the half-life of the Fc domain. Half-lives of 174-185 hrs were observed for constructs containing the FcRn ISVD (TP048 and TP050, respectively). compared to 134 hrs for the control construct (TP003). Antibodies with mutations I253A, H310A, H435A (IHH) in their Fc domain display dramatically reduced binding to FcRn and consequently very short in vivo half-lives (TP049).

Fusion of an FcRn-binding ISVD to an Fc modified to not bind to FcRn (IHH mutated Fc), reinstates a half-life which is in line with Fc therapeutics.

TABLE 20
Formats and calculated half-lives of ISVD-Fc
constructs evaluated in Tg32 mice.

| | TP003 | TP049 | TP048 | TP050 |
|---|---|---|---|---|
| Fc | IgG4 | IgG4(IHH) | IgG4 | IgG4 |
| T_{1/2} (hours) | 134 | 92.8 | 174 | 185 |

### 7. INDUSTRIAL APPLICATION

The present invention is capable of exploitation in industry. Applications and practical exploitation in industry may be derived from the present description by the skilled person's general knowledge.

1. A polypeptide comprising at least one immunoglobulin single variable domain (ISVD) specifically binding to an epitope on FcRn, characterized in that the epitope comprises at least one of the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 209Q, 255Q, 256H, 257A, 259L, 260A, 261Q, and/or 262P, amino acid residues being numbered according to SEQ ID NO: 1.

2. The polypeptide according to claim 1, characterized in that said epitope comprises at least one of the following combinations of amino acid residues:
a) 4H and 5L, and/or
b) 98L, 99G, 100P, 101D and 102N, and/or
c) 167L, 171R, 174L, 175E and 177K, and/or
d) 255Q, 256H, 257A, 259L, 260A and 262P,
amino acid residues being numbered according to SEQ ID NO: 1.

3. The polypeptide according to claim 1, characterized in that said epitope comprises at least one of the following combinations of amino acid residues:
a) 2E, 3S, 4H and 5L, and/or
b) 97E, 98L, 99G, 100P, 101D and 102N, and/or
c) 98L, 99G, 100P, 101D, 102N and 103T, and/or
e) 167L, 168E, 171R, 174L, 175E and 177K, and/or
d) 205P, 206P and 207E, and/or
e) 255Q, 256H, 257A, 259L, 260A, 261Q, and 262P,
amino acid residues being numbered according to SEQ ID NO: 1.

4. The polypeptide according to claim 1, characterized in that said epitope comprises at least one of the following combinations of amino acid residues:
a) 1A, 2E, 3S, 4H and 5L, and/or
b) 164R, 167L, 168E, 171R, 174L, 175E and 177K, and/or
c) 204Y, 205P, 206P and 230E, and/or
d) 205P, 206P, 207E and 208L,
amino acid residues being numbered according to SEQ ID NO: 1.

5. The polypeptide according to claim 1, characterized in that said epitope at least comprises the following amino acid residues 4H, 5L, 98L, 99G, 100P, 101D, 102N, 167L, 171R, 174L, 175E, 177K, 207E, 255Q, 256H, 257A, 259L, 260A, and 262P, amino acid residues being numbered according to SEQ ID NO: 1.

6. The polypeptide according to claim 1, characterized in that said epitope at least comprises the following amino acid residues 2E, 3S, 4H, 5L, 32P, 97E, 98L, 99G, 100P, 101D, 102N, 103T, 167L, 168E, 171R, 174L, 175E, 177K, 205P, 206P, 207E, 255Q, 256H, 257A, 259L, 260A, 261Q, and 262P, amino acid residues being numbered according to SEQ ID NO: 1, optionally wherein said epitope at least comprises the following amino acid residues 1A, 2E, 3S, 4H, 5L, 32P, 97E, 98L, 99G, 100P, 101D, 102N, 103T, 164R, 167L, 168E, 171R, 174L, 175E, 177K, 204Y, 205P, 206P, 207E, 208L, 2090, 2550, 256H, 257A, 259L, 260A, 2610, and 262P amino acid residues being numbered according to SEQ ID NO: 1.

7. (canceled)

8. The polypeptide according to claim 1, characterized in that said at least one immunoglobulin single variable domain (ISVD) specifically binds to said epitope on FcRn in a pH-dependent manner, such that the binding affinity at a pH between 5.0 and 6.8 is at least three times higher than the binding affinity at a pH of 7.4.

9. The polypeptide according to claim 1, characterized in that said at least one ISVD specifically binding to said epitope on FcRn specifically binds to amino acid residues on FcRn that are not involved in binding of FcRn to serum albumin and/or that are not involved in binding of FcRn to IgG.

10. The polypeptide according to claim 1, wherein said at least one ISVD specifically binding to said epitope on FcRn consists of 4 framework regions (FR1 to FR4 respectively) and 3 complementarity determining regions (CDR1 to CDR3 respectively) and is characterized in that:
a) CDR1 (according to AbM) has the amino acid sequence of SEQ ID NO: 11 [=G F T F S S Y A M Y] and/or has an amino acid sequence having 4, 3, 2 or only 1 “amino acid difference(s)” (as defined herein) with the sequence of SEQ ID NO: 11; and
b) CDR2 (according to AbM) has the amino acid sequence of SEQ ID NO: 12 [=A I S S G G G S T D] and/or has an amino acid sequence having 4, 3, 2 or only 1 “amino acid difference(s)” (as defined herein) with the sequence of SEQ ID NO: 12; and
c) CDR3 (according to AbM) has the amino acid sequence of SEQ ID NO: 13 [=D T L Y T S L T S Y S Y], and/or has an amino acid sequence having 4, 3, 2 or only 1 “amino acid difference(s)” (as defined herein) with the sequence of SEQ ID NO: 13.

11. The polypeptide according to claim 1, characterized in that said at least one ISVD specifically binding to said epitope on FcRn has the sequence of SEQ ID NO: 14 or SEQ ID NO: 15.

12. The polypeptide according to claim 1, characterized in that said polypeptide further comprises at least one ISVD specifically binding to (human) serum albumin.

13. The polypeptide according to claim 12, wherein said at least one ISVD specifically binding to serum albumin consists of 4 framework regions (FR1 to FR4 respectively) and 3 complementarity determining regions (CDR1 to CDR3 respectively), and is characterized in that: CDR1 is SFGMS (SEQ ID NO: 16), CDR2 is SISGSGSDTLYADSVKG (SEQ ID NO:
17) and CDR3 is GGSLSR (SEQ ID NO: 18), CDR determined according to Kabat definition;
and/or in which CDR1 is GFTFRSFGMS (SEQ ID NO: 19), CDR2 is SISGSGSDTL (SEQ ID NO: 20) and CDR3 is GGSLSR (SEQ ID NO: 21), CDR determined according to AbM definition.

14. The polypeptide according to claim 1, characterized in that said polypeptide further comprises an Fc region of an immunoglobulin (Ig).

15. The polypeptide according to claim 1, characterized in that said polypeptide further comprises at least one ISVD specifically binding to a therapeutic target.

16. A method for prolonging the in vivo half-life of a therapeutic or diagnostic compound comprising fusing or binding the polypeptide according to claim 1 to the therapeutic or diagnostic compound.

17. A pharmaceutical composition comprising a polypeptide according to claim 1.

18. A method for producing a polypeptide according to claim 1, said method at least comprising the steps of:
a. expressing, in a suitable host cell or host organism or in another suitable expression system, a nucleic acid sequence encoding the polypeptide; optionally followed by:
b. isolating and/or purifying the polypeptide.

19. A nucleic acid sequence encoding a polypeptide according to claim 1.

20. A non-human host or host cell comprising a vector comprising a nucleic acid sequence according to claim 19.

21. A method of treating a disease or disorder comprising administering, to a subject in need thereof, a pharmaceutically active amount of the polypeptide according to claim 1.


SEQUENCE LISTING (129 sequences)

SEQ ID NO: 1 - protein, 274 aa, Homo sapiens
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 2 - skipped

SEQ ID NO: 3 - protein, 17 aa, synthetic construct
  VARIANT 3: note = A, D, E, G, H, P, Q, R, S, T, or V
  VARIANT 4: note = A, D, E, G, K, M, N, P, Q, R, S, T, V, W, or Y
  VARIANT 8: note = A, D, G, H, K, L, M, Q, S, T, or V
  VARIANT 9: note = A, D, E, F, G, H, I, K, L, M, N, P, Q, R, S, T, V, W, or Y
  VARIANT 10: note = A, D, E, H, S, T, V, or Y
AIXXGGGXXXYADSVKG

SEQ ID NO: 4 - protein, 12 aa, synthetic construct
  VARIANT 2: note = A, D, E, F, G, I, L, M, Q, S, T, V, W, or Y
  VARIANT 3: note = L or N
  VARIANT 4: note = F, L, W, or Y
  VARIANT 6: note = A, D, E, K, L, M, Q, S, or W
  VARIANT 7: note = L, M, or V
  VARIANT 9: note = A, D, E, F, G, H, K, L, M, Q, R, S, T, V, W or Y
  VARIANT 11: note = A, E, L, M, Q, S, T, V, or W
  VARIANT 12: note = T or Y
DXXXTXXTXYXX

SEQ ID NO: 5 - skipped

SEQ ID NO: 6 - protein, 10 aa, synthetic construct
  VARIANT 3: note = A, D, E, G, H, P, Q, R, S, T, or V
  VARIANT 4: note = A, D, E, G, K, M, N, P, Q, R, S, T, V, W, or Y
  VARIANT 8: note = A, D, G, H, K, L, M, Q, S, T, or V
  VARIANT 9: note = A, D, E, F, G, H, I, K, L, M, N, P, Q, R, S, T, V, W, or Y
  VARIANT 10: note = A, D, E, H, S, T, V, or Y
AIXXGGGXXX

SEQ ID NO: 7 - protein, 12 aa, synthetic construct
  VARIANT 2: note = A, D, E, F, G, I, L, M, Q, S, T, V, W, or Y
  VARIANT 3: note = L or N
  VARIANT 4: note = F, L, W, or Y
  VARIANT 6: note = A, D, E, K, L, M, Q, S, or W
  VARIANT 7: note = L, M, or V
  VARIANT 9: note = A, D, E, F, G, H, K, L, M, Q, R, S, T, V, W or Y
  VARIANT 11: note = A, E, L, M, Q, S, T, V, or W
  VARIANT 12: note = T or Y
DXXXTXXTXYXX

SEQ ID NO: 8 - protein, 5 aa, synthetic construct
SYAMY

SEQ ID NO: 9 - protein, 17 aa, synthetic construct
AISSGGGSTDYADSVKG

SEQ ID NO: 10 - protein, 12 aa, synthetic construct
DTLYTSLTSYSY

SEQ ID NO: 11 - protein, 10 aa, synthetic construct
GFTFSSYAMY

SEQ ID NO: 12 - protein, 10 aa, synthetic construct
AISSGGGSTD

SEQ ID NO: 13 - protein, 12 aa, synthetic construct
DTLYTSLTSYSY

SEQ ID NO: 14 - protein, 121 aa, synthetic construct
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTWYSYWGQGTLVTVSS

SEQ ID NO: 15 - protein, 121 aa, synthetic construct
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 16 - protein, 5 aa, synthetic construct
SFGMS

SEQ ID NO: 17 - protein, 17 aa, synthetic construct
SISGSGSDTLYADSVKG

SEQ ID NO: 18 - protein, 6 aa, synthetic construct
GGSLSR

SEQ ID NO: 19 - protein, 10 aa, synthetic construct
GFTFRSFGMS

SEQ ID NO: 20 - protein, 10 aa, synthetic construct
SISGSGSDTL

SEQ ID NO: 21 - protein, 6 aa, synthetic construct
GGSLSR

SEQ ID NO: 22 - protein, 115 aa, synthetic construct [sequence omitted]

SEQ ID NO: 23 - protein, 115 aa, synthetic construct [sequence omitted]

SEQ ID NO: 24 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 25 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 26 - protein, 115 aa, synthetic construct [sequence omitted]

SEQ ID NO: 27 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 28 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 29 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 30 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 31 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 32 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 33 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 34 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 35 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 36 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 37 - skipped

SEQ ID NO: 38 - protein, 5 aa, synthetic construct
GGGGS

SEQ ID NO: 39 - protein, 7 aa, synthetic construct
SGGSGGS

SEQ ID NO: 40 - protein, 8 aa, synthetic construct
GGGGSGGS

SEQ ID NO: 41 - protein, 9 aa, synthetic construct
GGGGSGGGS

SEQ ID NO: 42 - protein, 10 aa, synthetic construct
GGGGSGGGGS

SEQ ID NO: 43 - protein, 15 aa, synthetic construct
GGGGSGGGGSGGGGS

SEQ ID NO: 44 - protein, 18 aa, synthetic construct
GGGGSGGGGSGGGGSGGS

SEQ ID NO: 45 - protein, 20 aa, synthetic construct
GGGGSGGGGSGGGGSGGGGS

SEQ ID NO: 46 - protein, 25 aa, synthetic construct
GGGGSGGGGSGGGGSGGGGSGGGGS

SEQ ID NO: 47 - protein, 30 aa, synthetic construct
GGGGSGGGGSGGGGSGGGGSGGGGSGGGGS

SEQ ID NO: 48 - protein, 35 aa, synthetic construct
GGGGSGGGGSGGGGSGGGGSGGGGSGGGGSGGGGS

SEQ ID NO: 49 - protein, 40 aa, synthetic construct
GGGGSGGGGSGGGGSGGGGSGGGGSGGGGSGGGGSGGGGS

SEQ ID NO: 50 - protein, 15 aa, synthetic construct
EPKSCDKTHTCPPCP

SEQ ID NO: 51 - protein, 24 aa, synthetic construct
GGGGSGGGSEPKSCDKTHTCPPCP

SEQ ID NO: 52 - protein, 12 aa, synthetic construct
EPKTPKPQPAAA

SEQ ID NO: 53 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 54 - protein, 4 aa, synthetic construct
KERE

SEQ ID NO: 55 - protein, 4 aa, synthetic construct
KQRE

SEQ ID NO: 56 - protein, 4 aa, synthetic construct
GLEW

SEQ ID NO: 57 - protein, 4 aa, synthetic construct
KQRE

SEQ ID NO: 58 - protein, 5 aa, synthetic construct
KEREL

SEQ ID NO: 59 - protein, 5 aa, synthetic construct
KEREF

SEQ ID NO: 60 - protein, 5 aa, synthetic construct
KQREL

SEQ ID NO: 61 - protein, 5 aa, synthetic construct
KQREF

SEQ ID NO: 62 - protein, 5 aa, synthetic construct
KEREG

SEQ ID NO: 63 - protein, 5 aa, synthetic construct
KQREW

SEQ ID NO: 64 - protein, 5 aa, synthetic construct
KQREG

SEQ ID NO: 65 - protein, 4 aa, synthetic construct
TERE

SEQ ID NO: 66 - protein, 5 aa, synthetic construct
TEREL

SEQ ID NO: 67 - protein, 4 aa, synthetic construct
TQRE

SEQ ID NO: 68 - protein, 5 aa, synthetic construct
TQREL

SEQ ID NO: 69 - protein, 4 aa, synthetic construct
KECE

SEQ ID NO: 70 - protein, 5 aa, synthetic construct
KECEL

SEQ ID NO: 71 - protein, 5 aa, synthetic construct
KECER

SEQ ID NO: 72 - protein, 4 aa, synthetic construct
KQCE

SEQ ID NO: 73 - protein, 5 aa, synthetic construct
KQCEL

SEQ ID NO: 74 - protein, 4 aa, synthetic construct
RERE

SEQ ID NO: 75 - protein, 5 aa, synthetic construct
REREG

SEQ ID NO: 76 - protein, 4 aa, synthetic construct
RQRE

SEQ ID NO: 77 - protein, 5 aa, synthetic construct
RQREL

SEQ ID NO: 78 - protein, 5 aa, synthetic construct
RQREF

SEQ ID NO: 79 - protein, 5 aa, synthetic construct
RQREW

SEQ ID NO: 80 - protein, 4 aa, synthetic construct
QERE

SEQ ID NO: 81 - protein, 5 aa, synthetic construct
QEREG

SEQ ID NO: 82 - protein, 4 aa, synthetic construct
QQRE

SEQ ID NO: 83 - protein, 5 aa, synthetic construct
QQREW

SEQ ID NO: 84 - protein, 5 aa, synthetic construct
QQREL

SEQ ID NO: 85 - protein, 5 aa, synthetic construct
QQREF

SEQ ID NO: 86 - protein, 4 aa, synthetic construct
KGRE

SEQ ID NO: 87 - protein, 4 aa, synthetic construct
KGRE

SEQ ID NO: 88 - protein, 5 aa, synthetic construct
KGREG

SEQ ID NO: 89 - protein, 4 aa, synthetic construct
KDRE

SEQ ID NO: 90 - protein, 5 aa, synthetic construct
KDREV

SEQ ID NO: 91 - protein, 5 aa, synthetic construct
DECKL

SEQ ID NO: 92 - protein, 5 aa, synthetic construct
NVCEL

SEQ ID NO: 93 - protein, 4 aa, synthetic construct
GVEW

SEQ ID NO: 94 - protein, 4 aa, synthetic construct
GLER

SEQ ID NO: 95 - protein, 4 aa, synthetic construct
DQEW

SEQ ID NO: 96 - protein, 4 aa, synthetic construct
DLEW

SEQ ID NO: 97 - protein, 4 aa, synthetic construct
GIEW

SEQ ID NO: 98 - protein, 4 aa, synthetic construct
ELEW

SEQ ID NO: 99 - protein, 4 aa, synthetic construct
GPEW

SEQ ID NO: 100 - protein, 4 aa, synthetic construct
EWLP

SEQ ID NO: 101 - protein, 4 aa, synthetic construct
GPER

SEQ ID NO: 102 - protein, 4 aa, synthetic construct
GLER

SEQ ID NO: 103 - protein, 4 aa, synthetic construct
ELEW

SEQ ID NO: 104 - protein, 12 aa, synthetic construct
DTLYTSLTWYSY

SEQ ID NO: 105 - protein, 12 aa, synthetic construct
DTLYTSLTWYSY

SEQ ID NO: 106 - protein, 30 aa, synthetic construct
EVQLVESGGGVVQPGGSLRLSCAASGFTFS

SEQ ID NO: 107 - protein, 14 aa, synthetic construct
WVRQAPGKGLEWVS

SEQ ID NO: 108 - protein, 32 aa, synthetic construct
RFTISRDNSKNTLYLQMNSLRPEDTALYYCAA

SEQ ID NO: 109 - protein, 11 aa, synthetic construct
WGQGTLVTVSS

SEQ ID NO: 110 - protein, 30 aa, synthetic construct
EVQLVESGGGVVQPGGSLRLSCAASGFTFR

SEQ ID NO: 111 - protein, 14 aa, synthetic construct
WVRQAPGKGPEWVS

SEQ ID NO: 112 - protein, 32 aa, synthetic construct
RFTISRDNSKNTLYLQMNSLRPEDTALYYCTI

SEQ ID NO: 113 - protein, 11 aa, synthetic construct
SSQGTLVTVSS

SEQ ID NO: 114 - protein, 25 aa, synthetic construct
EVQLVESGGGVVQPGGSLRLSCAAS

SEQ ID NO: 115 - protein, 14 aa, synthetic construct
WVRQAPGKGLEWVS

SEQ ID NO: 116 - protein, 39 aa, synthetic construct
YADSVKGRFTISRDNSKNTLYLQMNSLRPEDTALYYCAA

SEQ ID NO: 117 - protein, 11 aa, synthetic construct
WGQGTLVTVSS

SEQ ID NO: 118 - protein, 25 aa, synthetic construct
EVQLVESGGGVVQPGGSLRLSCAAS

SEQ ID NO: 119 - protein, 18 aa, synthetic construct
FGMSWVRQAPGKGPEWVS

SEQ ID NO: 120 - protein, 39 aa, synthetic construct
YADSVKGRFTISRDNSKNTLYLQMNSLRPEDTALYYCTI

SEQ ID NO: 121 - protein, 11 aa, synthetic construct
SSQGTLVTVSS

SEQ ID NO: 122 - protein, 271 aa, synthetic construct
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTWYSYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 123 - protein, 271 aa, synthetic construct
EVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 124 - protein, 271 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTWYSYWGQGTLVTVSS

SEQ ID NO: 125 - protein, 271 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSGGGGSGGGGSGGGGSGGGGSEVQLVESGGGVVQPGGSLRLSCAASGFTFSSYAMYWVRQAPGKGLEWVSAISSGGGSTDYADSVKGRFTISRDNSKNTLYLQMNSLRPEDTALYYCAADTLYTSLTSYSYWGQGTLVTVSS

SEQ ID NO: 126 - protein, 10 aa, synthetic construct
DKTHTCPPCP

SEQ ID NO: 127-128 - skipped (2 entries)

SEQ ID NO: 129 - protein, 396 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA